(19) United States
(12) Patent Application Publication
(10) Pub. No.: US 2010/0263088 A1
(43) Pub. Date: Oct. 14, 2010

(54) Promoters From Brassica Napus For Seed Specific Gene Expression

(75) Inventors: Jörg Bauer, Limburgerhof (DE); Tom Wetjen, Mannheim (DE); Xiao Qiu, Saskatoon (CA); Guohai Wu, Saskatoon (CA)

Correspondence Address: CONNOLLY BOVE LODGE & HUTZ, LLP, P O BOX 2207, WILMINGTON, DE, 19899, US

(73) Assignees: BASF Plant Science GmbH; Bioriginal Food & Science Corp

(21) Appl. No.: 12/747,670

(22) PCT Filed: Dec. 15, 2008
(86) PCT No.: PCT/EP08/67492

§ 371 Date: Jun. 11, 2010

(30) Foreign Application Priority Data
Dec. 14, 2007 (EP) 07150029.2

(51) Int. Cl. A01H 5/10; C07H 21/04; C12N 15/82; C12N 5/10; A01H 5/00

(52) U.S. Cl. 800/278; 536/23.1; 435/320.1; 435/419; 800/298

(57) ABSTRACT

The present invention is concerned with means and methods for allowing tissue specific and, in particular, seed specific expression of genes. The present invention, accordingly, relates to a polynucleotide comprising an expression control sequence which allows seed specific expression of a nucleic acid of interest being operatively linked thereto. Moreover, the present invention contemplates vectors, host cells, non-human transgenic organisms comprising the aforementioned polynucleotide as well as methods and uses of such a polynucleotide.

The present invention is concerned with means and methods for allowing tissue specific and, in particular, seed specific expression of genes. The present invention, accordingly, relates to a polynucleotide comprising an expression control sequence which allows seed specific expression of a nucleic acid of interest being operatively linked thereto. Moreover, the present invention contemplates vectors, host cells, non-human transgenic organisms comprising the aforementioned polynucleotide as well as methods and uses of such a polynucleotide.

In the field of “green” (agricultural) biotechnology, plants are genetically manipulated in order to confer beneficial traits. These beneficial traits may be yield increase, tolerance increase, reduced dependency on fertilizers, herbicidal, pesticidal- or fungicidal-resitance, or the capability of producing chemical specialties such as nutrients, drugs, oils for food and petrochemistry etc.

In many cases, it is required to express a heterologous gene in the genetically modified plants at a rather specific location in order to obtain a plant exhibiting the desired beneficial trait. One major location for gene expression is the plant seed. In the seeds, many important synthesis pathways, e.g., in fatty acid synthesis, take place. Accordingly, expression of heterologous genes in seeds allow for the manipulation of fatty acid synthesis pathways and, thus, for the provision of various fatty acid derivatives and lipid-based compounds.

However, for many heterologous genes, a seed specific expression will be required. Promoters which allow for a seed specific expression are known in the art. Such promoters include the oilseed rape napin promoter (U.S. Pat. No. 5,608,152), the Vicia faba USP promoter (Baeumlein et al., Mol Gen Genet, 1991, 225 (3):459-67), the Arabidopsis oleosin promoter (WO 98/45461), the Phaseolus vulgaris phaseolin promoter (U.S. Pat. No. 5,504,200), the Brassica Bce4 promoter (WO 91/13980) or the legumine B4 promoter (LeB4; Baeumlein et al., 1992, Plant Journal, 2 (2):233-9), and promoters which bring about the seed-specific expression in monocotyledonous plants such as maize, barley, wheat, rye, rice and the like. Suitable noteworthy promoters are the barley Ipt2 or Ipt1 gene promoter (WO 95/15389 and WO 95/23230) or the promoters from the barley hordein gene, the rice glutelin gene, the rice oryzin gene, the rice prolamine gene, the wheat gliadine gene, the wheat glutelin gene, the maize zeine gene, the oat glutelin gene, the sorghum kasirin gene or the rye secalin gene, which are described in WO 99/16890.

However, there is a clear need for further promoters which allow for a reliable and efficient expression of foreign nucleic acids in seeds.

The technical problem underlying this invention can be seen as the provision of means and methods complying with the aforementioned needs. The technical problem is solved by the embodiments characterized in the claims and herein below.

Accordingly, the present invention relates to a polynucleotide comprising an expression control sequence which allows seed specific expression of a nucleic acid of interest being operatively linked thereto, said expression control sequence being selected from the group consisting of:
(a) an expression control sequence having a nucleic acid sequence as shown in any one of SEQ ID NOs: 7 to 12;
(b) an expression control sequence having a nucleic acid sequence which hybridizes under stringent conditions to a a nucleic acid sequence as shown in any one of SEQ ID NOs: 7 to 12;
(c) an expression control sequence having a nucleic acid sequence which hybridizes to a nucleic acid sequences located upstream of an open reading frame sequence shown in any one of SEQ ID NOs: 1 to 6;
(d) an expression control sequence having a nucleic acid sequence which hybridizes to a nucleic acid sequences located upstream of an open reading frame sequence being at least 80% identical to an open reading frame sequence as shown in any one of SEQ ID NOs: 1 to 6;
(e) an expression control sequence obtainable by 5′ genome walking from an open reading frame sequence as shown in any one of SEQ ID NOs: 1 to 6; and
(f) an expression control sequence obtainable by 5′ genome walking from an open reading frame sequence being at least 80% identical to an open reading frame as shown in any one of SEQ ID NOs: 1 to 6.

The term “polynucleotide” as used herein refers to a linear or circular nucleic acid molecule. It encompasses DNA as well as RNA molecules. The polynucleotide of the present invention is characterized in that it shall comprise an expression control sequence as defined elsewhere in this specification. In addition to the expression control sequence, the polynucleotide of the present invention, preferably, further comprises at least one nucleic acid of interest being operatively linked to the expression control sequence and/or a termination sequence for transcription. Thus, the polynucleotide of the present invention, preferably, comprises an expression cassette for the expression of at least one nucleic acid of interest. Alternatively, the polynucleotide may comprise in addition to the said expression control sequence a multiple cloning site and/or a termination sequence for transcription. In such a case, the multiple cloning site is, preferably, arranged in a manner as to allow for operative linkage of a nucleic acid to be introduced in the multiple cloning site with the expression control sequence. In addition to the aforementioned components, the polynucleotide of the present invention, preferably, could comprise components required for homologous recombination, i.e. flanking genomic sequences from a target locus. However, also preferably, the polynucleotide of the present invention can essentially consist of the said expression control sequence.

The term “expression control sequence” as used herein refers to a nucleic acid which is capable of governing the expression of another nucleic acid operatively linked thereto, e.g. a nucleic acid of interest referred to elsewhere in this specification in detail. An expression control sequence as referred to in accordance with the present invention, preferably, comprises sequence motifs which are recognized and bound by polypeptides, i.e. transcription factors. The said transcription factors shall upon binding recruit RNA polymerases, preferably, RNA polymerase I, II or III, more preferably, RNA polymerase II or III, and most preferably, RNA polymerase II. Thereby the expression of a nucleic acid operatively linked to the expression control sequence will be initiated. It is to be understood that dependent on the type of nucleic acid to be expressed, i.e. the nucleic acid of interest, expression as meant herein may comprise transcription of RNA polynucleotides from the nucleic acid sequence (as suitable for, e.g., anti-sense approaches or RNAi approaches) or may comprises transcription of RNA polynucleotides followed by translation of the said RNA polynucleotides into polypeptides (as suitable for, e.g., gene expression and recombinant polypeptide production approaches). In order to govern expression of a nucleic acid, the expression control sequence may be located immediately adjacent to the nucleic acid to be expressed, i.e. physically linked to the said nucleic acid at its 5′ end. Alternatively, it may be located in physical proximity. In the latter case, however, the sequence must be located so as to allow functional interaction with the nucleic acid to be expressed. An expression control sequence referred to herein, preferably, comprises between 200 and 5,000 nucleotides in length. More preferably, it comprises between 500 and 2,500 nucleotides and, more preferably, at least 1,000 nucleotides. As mentioned before, an expression control sequence, preferably, comprises a plurality of sequence motifs which are required for transcription factor binding or for conferring a certain structure to the polynucletide comprising the expression control sequence. Sequence motifs are also sometimes referred to as cis-regulatory elements and, as meant herein, include promoter elements as well as enhancer elements.

Preferred expression control sequences to be included into a polynucleotide of the present invention have a nucleic acid sequence as shown in any one of SEQ ID NOs: 7 to 12.

Further preferably, an expression control sequence comprised by a polynucleotide of the present invention has a nucleic acid sequence which hybridizes to a nucleic acid sequences located upstream of an open reading frame sequence shown in any one of SEQ ID NOs: 1 to 6, i.e. is a variant expression control sequence. It will be understood that expression control sequences may slightly differ in its sequences due to allelic variations. Accordingly, the present invention also contemplates an expression control sequence which can be derived from an open reading frame as shown in any one of SEQ ID NOs: 1 to 6. Said expression control sequences are capable of hybridizing, preferably under stringent conditions, to the upstream sequences of the open reading frames shown in any one of SEQ ID NOs. 1 to 6, i.e. the expression control sequences shown in any one of SEQ ID NOs.: 7 to 12. Stringent hybridization conditions as meant herein are, preferably, hybridization conditions in 6× sodium chloride/sodium citrate (=SSC) at approximately 45° C., followed by one or more wash steps in 0.2×SSC, 0.1% SDS at 53 to 65° C., preferably at 55° C., 56° C., 57° C., 58° C., 59° C., 60° C., 61° C., 62° C., 63° C., 64° C. or 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 by (=base pairs) in length and a G+C content of 50% in the absence of formamide. Such hybridizing expression control sequences are, more preferably, at least 70%, at least 80%, at least 90%, at least 91%, at least 92%, at least 93%, at least 94% at least 95%, at least 96%, at least 97%, at least 98%, or at least 99% identical to the expression control sequences as shown in any one of SEQ ID NOs.: 7 to 12. The percent identity values are, preferably, calculated over the entire nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit [Needleman and Wunsch (J. Mol. Biol. 48; 443-453 (1970)) and Smith and Waterman (Adv. Appl. Math. 2; 482-489 (1981))], which are part of the GCG software packet [Genetics Computer Group, 575 Science Drive, Madison, Wis., USA 53711 (1991)], are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

Moreover, expression control sequences which allow for seed specific expression can not only be found upstream of the aforementioned open reading frames having a nucleic acid sequence as shown in any one of SEQ ID NOs. 1 to 6. Rather, expression control sequences which allow for seed specific expression can also be found upstream of orthologous, paralogous or homologous genes (i.e. open reading frames). Thus, also preferably, an variant expression control sequence comprised by a polynucleotide of the present invention has a nucleic acid sequence which hybridizes to a nucleic acid sequences located upstream of an open reading frame sequence being at least 70%, more preferably, at least 80%, at least 90%, at least 91%, at least 92%, at least 93%, at least 94% at least 95%, at least 96%, at least 97%, at least 98%, or at least 99% identical to a sequence as shown in any one of SEQ ID NOs: 1 to 6. The said variant open reading shall encode a polypeptide having the biological activity of the corresponding polypeptide being encoded by the open reading frame shown in any one of SEQ ID NOs.: 1 to 6. In this context it should be mentioned that the open reading frame shown in SEQ ID NO: 1 encodes a polypeptide having pectinesterase activity, the open reading frames shown in SEQ ID NO: 2 and 5 encode “late embryogenesis adundant” (LEA) polypeptides, the open reading frame shown in SEQ ID NO: 3 encodes a polypeptide having anthocyanidin reductase activity, the open reading frame shown in SEQ ID NO: 4 encodes a polypeptide having proteinase inhibitor activity, and the open reading frame shown in SEQ ID NO: 6 encodes a polypeptide having lipid transfer activity. These biological activities can be determined by those skilled in the art without further ado.

Also preferably, a variant expression control sequence comprised by a polynucleotide of the present invention is (i) obtainable by 5′ genome walking from an open reading frame sequence as shown in any one of SEQ ID NOs: 1 to 6 or (ii) obtainable by 5′ genome walking from a open reading frame sequence being at least 80% identical to an open reading frame as shown in any one of SEQ ID NOs: 1 to 6. Variant expression control sequences are obtainable without further by the genome walking technology which can be carried out as described in the accompanying Examples by using, e.g., commercially available kits.

Variant expression control sequences referred to in this specification for the expression control sequence shown in SEQ ID NO: 7, preferably, comprise at least 80, at least 90, at least 100, at least 110, at least 120 or all of the sequence motifs recited in Table 1. Variant expression control sequences referred to in this specification for the expression control sequence shown in SEQ ID NO: 8, preferably, comprise at least 80, at least 90, at least 100, at least 110, at least 120, at least 130, at least 140 or all of the sequence motifs recited in Table 2. Variant expression control sequences referred to in this specification for the expression control sequence shown in SEQ ID NO: 9, preferably, comprise at least 80, at least 90, at least 100, at least 110 or all of the sequence motifs recited in Table 3. Variant expression control sequences referred to in this specification for the expression control sequence shown in SEQ ID NO: 10, preferably, comprise at least 40, at least 50, at least 60, at least 70 or all of the sequence motifs recited in Table 4. Variant expression control sequences referred to in this specification for the expression control sequence shown in SEQ ID NO: 11, preferably, comprise at least 80, at least 150, at least 200, at least 210, at least 220, at least 230, at least 240 or all of the sequence motifs recited in Table 5. Variant expression control sequences referred to in this specification for the expression control sequence shown in SEQ ID NO: 12, preferably, comprise at least 80, at least 90, at least 100, at least 110, at least 120 or all of the sequence motifs recited in Table 6. Specifically, the following elements are preferably comprised by all variant expression control sequences referred to in accordance with the present invention: CA-rich element, CCAAT box, G-box binding factor 1, RY repeat element, Prolamin box legumin box, Dof box and RITA motif. The specific sequnces for the elements are shown in the Tables, below (marked in bold). These elements are characteristic for seed-specific promoters (Kim 2006, Mol Genet Genomics 276(4):351-368).

The term “seed specific” as used herein means that a nucleic acid of interest being operatively linked to the expression control sequence referred to herein will be predominantly expressed in seeds when present in a plant. A predominant expression as meant herein is characterized by a statistically significantly higher amount of detectable transcription in the seeds with respect to other plant tissues. A statistically significant higher amount of transcription is, preferably, an amount being at least two-fold, three-fold, four-fold, five-fold, ten-fold, hundred-fold, five hundred-fold or thousand-fold the amount found in at least one of the other tissues with detectable transcription. Alternatively, it is an expression in seeds whereby the amount of transcription in non-seed tissues is less than 1%, 2%, 3%, 4% or most preferably 5% of the overall (whole plant) amount of expression. The amount of transcription directly correlates to the amount of transcripts (i.e. RNA) or polypeptides encoded by the transcripts present in a cell or tissue. Suitable techniques for measuring transcription either based on RNA or polypeptides are well known in the art. Seed specific alternatively and, preferably in addition to the above, means that the expression is restricted or almost restricted to seeds, i.e. there is essentially no detectable transcription in other tissues. Almost restricted as meant herein means that unspecific expression is detectable in less than ten, less than five, less than four, less than three, less than two or one other tissue(s). Seed specific expression as used herein includes expression in seed cells or their precursors, such as cells of the endosperm and of the developing embryo.

An expression control sequences can be tested for seed specific expression by determining the expression pattern of a nucleic acid of interest, e.g., a nucleic acid encoding a reporter protein, such as GFP, in a transgenic plant. Transgenic plants can be generated by techniques well known to the person skilled in the art and as discussed elsewhere in this specification. The aforementioned amounts or expression pattern are, preferably, determined by Northern Blot or in situ hybridization techniques as described in WO 02/102970 in Brassica napus plants, most preferably, at 40 days after flowering.

The term “nucleic acid of interest” refers to a nucleic acid which shall be expressed under the control of the expression control sequence referred to herein. Preferably, a nucleic acid of interest encodes a polypeptide the presence of which is desired in a cell or non-human organism as referred to herein and, in particular, in a plant seed. Such a polypeptide may be an enzyme which is required for the synthesis of seed storage compounds or may be a seed storage protein. It is to be understood that if the nucleic acid of interest encodes a polypeptide, transcription of the nucleic acid in RNA and translation of the transcribed RNA into the polypeptide may be required. A nucleic acid of interest, also preferably, includes biologically active RNA molecules and, more preferably, antisense RNAs, ribozymes, micro RNAs or siRNAs. Said biologically active RNA molecules can be used to modify the amount of a target polypeptide present in a cell or non-human organism. For example, an undesired enzymatic activity in a seed can be reduced due to the seed specific expression of an antisense RNAs, ribozymes, micro RNAs or siRNAs. The underlying biological principles of action of the aforementioned biologically active RNA molecules are well known in the art. Moreover, the person skilled in the art is well aware of how to obtain nucleic acids which encode such biologically active RNA molecules. It is to be understood that the biologically active RNA molecules may be directly obtained by transcription of the nucleic acid of interest, i.e. without translation into a polypeptide. It is to be understood that the expression control sequence may also govern the expression of more than one nucleic acid of interest, i.e. at least one, at least two, at least three, at least four, at least five etc. nucleic acids of interest.

The term “operatively linked” as used herein means that the expression control sequence of the present invention and a nucleic acid of interest, are linked so that the expression can be governed by the said expression control sequence, i.e. the expression control sequence shall be functionally linked to said nucleic acid sequence to be expressed. Accordingly, the expression control sequence and the nucleic acid sequence to be expressed may be physically linked to each other, e.g., by inserting the expression control sequence at the 5′ end of the nucleic acid sequence to be expressed. Alternatively, the expression control sequence and the nucleic acid to be expressed may be merely in physical proximity so that the expression control sequence is capable of governing the expression of the at least one nucleic acid sequence of interest. The expression control sequence and the nucleic acid to be expressed are, preferably, separated by not more than 500 bp, 300 bp, 100 bp, 80 bp, 60 bp, 40 bp, 20 bp, 10 by or 5 bp.

As set forth above, the polynucleotide of the present invention, in a preferred embodiment, comprises also a termination sequence for transcription downstream of the nucleic acid of interest. A termination sequence for transcription relates to a nucleic acid sequence which terminates the process of RNA transcription. Suitable termination sequences are well known in the art and comprise, preferably, the SV40-poly-A site, the tk-poly-A site, the nos or ocs terminator from Agrobacterium tumefaciens or the 35S terminator from Cauliflower mosaic virus.

Advantageously, it has been found in the studies underlying the present invention that seed specific expression of a nucleic acid of interest can be achieved by expressing said nucleic acid of interest under the control of an expression control sequence from Brassica napus or a variant expression control sequence as specified above. The expression control sequences provided by the present invention allow for a reliable and highly specific expression of nucleic acids of interest. Thanks to the present invention, it is possible to (i) specifically manipulate biochemical processes in seeds, e.g., by expressing heterologous enzymes or biologically active RNAs, or (ii) to produce heterologous proteins in seeds. In principle, the present invention contemplates the use of the polynucleotide, the vector, the host cell or the non-human transgenic organism for the expression of a nucleic acid of interest. Preferably, the envisaged expression is seed specific. More preferably, the nucleic acid of interest to be used in the various embodiments of the present invention encodes a seed storage protein or is involved in the modulation of seed storage compounds.

As used herein, seed storage compounds include fatty acids and triacylglycerides which have a multiplicity of applications in the food industry, in animal nutrition, in cosmetics and the pharmacological sector. Depending on whether they are free saturated or unsaturated fatty acids or else triacylglycerides with an elevated content of saturated or unsaturated fatty acids, they are suitable for various different applications. More preferably, the polynucleotide of the present invention comprising the expression control sequence referred to above is applied for the manufacture of polyunsaturated fatty acids (PUFAs). For the manufacture of PUFAs in seeds, the activity of enzymes involved in their synthesis, in particular, elongases and desaturases, needs to be modulated. This will be achieved by seed specific expression of the nucleic acids of interest encoding the aforementioned enzymes or by seed specific expression of antisense, ribozyme, RNAi molecules which downregulate the activity of the enzymes by interfering with their protein synthesis. PUFAs are seed storage compounds which can be isolated by a subsequently applied purification process using the aforementioned seeds.

Particularly preferred PUFAs in accordance with the present invention are polyunsaturated long-chain ω-3-fatty acids such as eicosapentaenoic acid (=EPA, C20:5^{Δ5,8,11,14,17}), ω-3 eicostetraenic acid (=ETA, C20:4^{Δ8,11,14,17}), arachidonic acid (=ARA C20:4^{Δ5,8,11,14}) or docosahexaenoic acid (=DHA, C22:6^{Δ4,7,10,13,16,19}). They are important components of human nutrition owing to their various roles in health aspects, including the development of the child brain, the functionality of the eyes, the synthesis of hormones and other signal substances, and the prevention of cardiovascular disorders, cancer and diabetes (Poulos, A Lipids 30:1-14, 14^{Δ8,11,14,17}995; Horrocks, L A and Yeo Y K Pharmacol Res 40:211-225, 1999). There is, therefore, a need for the production of polyunsaturated long-chain fatty acids.

Particular preferred enzymes involved in the synthesis of PUFAs are disclosed in WO 91/13972 (Δ9-desaturase), WO 93/11245 (Δ15-desaturase), WO 94/11516 (Δ12-desaturase), EP A 0 550 162, WO 94/18337, WO 97/30582, WO 97/21340, WO 95/18222, EP A 0 794 250, Stukey et al., J. Biol. Chem., 265, 1990: 20144-20149, Wada et al., Nature 347, 1990: 200-203 or Huang et al., Lipids 34, 1999: 649-659. Δ6-Desaturases are described in WO 93/06712, U.S. Pat. No. 5,614,393, U.S. Pat. No. 5,614,393, WO 96/21022, WO 00/21557 and WO 99/27111, and also the application for the production in transgenic organisms is described in WO 98/46763, WO 98/46764 and WO 98/46765. Here, the expression of various desaturases is also described and claimed in WO 99/64616 or WO 98/46776, as is the formation of polyunsaturated fatty acids. As regards the expression efficacy of desaturases and its effect on the formation of polyunsaturated fatty acids, it must be noted that the expression of a single desaturase as described to date has only resulted in low contents of unsaturated fatty acids/lipids such as, for example, γ-linolenic acid and stearidonic acid. Furthermore, mixtures of ω-3- and ω-6-fatty acids are usually obtained.

The present invention also relates to a vector comprising the polynucleotide of the present invention.

The term “vector”, preferably, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotides of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, natural competence, carbon-based clusters, chemically mediated transfer, electroporation or particle bombardment (e.g., “gene-gun”). Suitable methods for the transformation or transfection of host cells, including plant cells, can be found in Sambrook et al. (Molecular Cloning: A Laboratory Manual, 2^{nd} ed., Cold Spring Harbor Laboratory, Cold Spring Harbor Laboratory Press, Cold Spring Harbor, N.Y., 1989) and other laboratory manuals, such as Methods in Molecular Biology, 1995, Vol. 44, Agrobacterium protocols, Ed.: Gartland and Davey, Humana Press, Totowa, N.J. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells.

Preferably, the vector referred to herein is suitable as a cloning vector, i.e. replicable in microbial systems. Such vectors ensure efficient cloning in bacteria and, preferably, yeasts or fungi and make possible the stable transformation of plants. Those which must be mentioned are, in particular, various binary and co-integrated vector systems which are suitable for the T-DNA-mediated transformation. Such vector systems are, as a rule, characterized in that they contain at least the vir genes, which are required for the Agrobacterium-mediated transformation, and the sequences which delimit the T-DNA (T-DNA border). These vector systems, preferably, also comprise further cis-regulatory regions such as promoters and terminators and/or selection markers with which suitable transformed host cells or organisms can be identified. While co-integrated vector systems have vir genes and T-DNA sequences arranged on the same vector, binary systems are based on at least two vectors, one of which bears vir genes, but no T-DNA, while a second one bears T-DNA, but no vir gene. As a consequence, the last-mentioned vectors are relatively small, easy to manipulate and can be replicated both in E. coli and in Agrobacterium. These binary vectors include vectors from the pBIB-HYG, pPZP, pBecks, pGreen series. Preferably used in accordance with the invention are Bin19, pBI101, pBinAR, pGPTV and pCAMBIA. An overview of binary vectors and their use can be found in Hellens et al, Trends in Plant Science (2000) 5, 446-451. Furthermore, by using appropriate cloning vectors, the polynucleotide of the invention can be introduced into host cells or organisms such as plants or animals and, thus, be used in the transformation of plants, such as those which are published, and cited, in: Plant Molecular Biology and Biotechnology (CRC Press, Boca Raton, Fla.), chapter 6/7, pp. 71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205-225.

More preferably, the vector of the present invention is an expression vector. In such an expression vector, the polynucleotide comprises an expression cassette as specified above allowing for expression in eukaryotic cells or isolated fractions thereof. An expression vector may, in addition to the polynucleotide of the invention, also comprise further regulatory elements including transcriptional as well as translational enhancers. Preferably, the expression vector is also a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

Suitable expression vector backbones are, preferably, derived from expression vectors known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX (Pharmacia Biotech Inc; Smith, D. B., and Johnson, K. S. (1988) Gene 67:31-40), pMAL (New England Biolabs, Beverly, Mass.) and pRIT5 (Pharmacia, Piscataway, N.J.), where glutathione S-transferase (GST), maltose E-binding protein and protein A, respectively, are fused with the nucleic acid of interest encoding a protein to be expressed. The target gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a coexpressed viral RNA polymerase (T7 gn1). This viral polymerase is provided by the host strains BL21 (DE3) or HMS174 (DE3) from a resident λ-prophage which harbors a T7 gn1 gene under the transcriptional control of the lacUV 5 promoter. Examples of vectors for expression in the yeast S. cerevisiae comprise pYeDesaturasec1 (Baldari et al. (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.). Vectors and processes for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells (for example Sf9 cells) comprise the pAc series (Smith et al. (1983) Mol. Cell Biol. 3:2156-2165) and the pVL series (Lucklow and Summers (1989) Virology 170:31-39).

The polynucleotides of the present invention can be used for expression of a nucleic acid of interest in single-cell plant cells (such as algae), see Falciatore et al., 1999, Marine Biotechnology 1 (3):239-251 and the references cited therein, and plant cells from higher plants (for example Spermatophytes, such as arable crops) by using plant expression vectors. Examples of plant expression vectors comprise those which are described in detail in: Becker, D., Kemper, E., Schell, J., and Masterson, R. (1992) “New plant binary vectors with selectable markers located proximal to the left border”, Plant Mol. Biol. 20:1195-1197; and Bevan, M. W. (1984) “Binary Agrobacterium vectors for plant transformation”, Nucl. Acids Res. 12:8711-8721; Vectors for Gene Transfer in Higher Plants; in: Transgenic Plants, Vol. 1, Engineering and Utilization, Ed.: Kung and R. Wu, Academic Press, 1993, p. 15-38. A plant expression cassette, preferably, comprises regulatory sequences which are capable of controlling the gene expression in plant cells and which are functionally linked so that each sequence can fulfill its function, such as transcriptional termination, for example polyadenylation signals. Preferred polyadenylation signals are those which are derived from Agrobacterium tumefaciens T-DNA, such as the gene 3 of the Ti plasmid pTiACH5, which is known as octopine synthase (Gielen et al., EMBO J. 3 (1984) 835 et seq.) or functional equivalents of these, but all other terminators which are functionally active in plants are also suitable. Since plant gene expression is very often not limited to transcriptional levels, a plant expression cassette preferably comprises other functionally linked sequences such as translation enhancers, for example the overdrive sequence, which comprises the 5′-untranslated tobacco mosaic virus leader sequence, which increases the protein/RNA ratio (Gallie et al., 1987, Nucl. Acids Research 15:8693-8711). Other preferred sequences for the use in functional linkage in plant gene expression cassettes are targeting sequences which are required for targeting the gene product into its relevant cell compartment (for a review, see Kermode, Crit. Rev. Plant Sci. 15, 4 (1996) 285-423 and references cited therein), for example into the vacuole, the nucleus, all types of plastids, such as amyloplasts, chloroplasts, chromoplasts, the extracellular space, the mitochondria, the endoplasmic reticulum, oil bodies, peroxisomes and other compartments of plant cells.

The abovementioned vectors are only a small overview of vectors to be used in accordance with the present invention. Further vectors are known to the skilled worker and are described, for example, in: Cloning Vectors (Ed., Pouwels, P. H., et al., Elsevier, Amsterdam-New York-Oxford, 1985, ISBN 0 444 904018). For further suitable expression systems for prokaryotic and eukaryotic cells see the chapters 16 and 17 of Sambrook, J., Fritsch, E. F., and Maniatis, T., Molecular Cloning: A Laboratory Manual, 2^{nd} edition, Cold Spring Harbor Laboratory, Cold Spring Harbor Laboratory Press, Cold Spring Harbor, N.Y., 1989.

The present invention also contemplates a host cell comprising the polynucleotide or the vector of the present invention.

Host cells are primary cells or cell lines derived from multicellular organisms such as plants or animals. Furthermore, host cells encompass prokaryotic or eukaryotic single cell organisms (also referred to as micro-organisms). Primary cells or cell lines to be used as host cells in accordance with the present invention may be derived from the multicellular organisms referred to below. Host cells which can be exploited are furthermore mentioned in: Goeddel, Gene Expression Technology: Methods in Enzymology 185, Academic Press, San Diego, Calif. (1990). Specific expression strains which can be used, for example those with a lower protease activity, are described in: Gottesman, S., Gene Expression Technology: Methods in Enzymology 185, Academic Press, San Diego, Calif. (1990) 119-128. These include plant cells and certain tissues, organs and parts of plants in all their phenotypic forms such as anthers, fibers, root hairs, stalks, embryos, calli, cotelydons, petioles, harvested material, plant tissue, reproductive tissue and cell cultures which is derived from the actual transgenic plant and/or can be used for bringing about the transgenic plant. Preferably, the host cells may be obtained from plants. More preferably, oil crops are envisaged which comprise large amounts of lipid compounds, such as oilseed rape, evening primrose, hemp, thistle, peanut, canola, linseed, soybean, safflower, sunflower, borage, or plants such as maize, wheat, rye, oats, triticale, rice, barley, cotton, cassava, pepper, Tagetes, Solanaceae plants such as potato, tobacco, eggplant and tomato, Vicia species, pea, alfalfa, bushy plants (coffee, cacao, tea), Salix species, trees (oil palm, coconut) and perennial grasses and fodder crops. Especially preferred plants according to the invention are oil crops such as soybean, peanut, oilseed rape, canola, linseed, hemp, evening primrose, sunflower, safflower, trees (oil palm, coconut). Suitable methods for obtaining host cells from the multicellular organisms referred to below as well as conditions for culturing these cells are well known in the art.

The micro-organisms are, preferably, bacteria or fungi including yeasts. Preferred fungi to be used in accordance with the present invention are selected from the group of the families Chaetomiaceae, Choanephoraceae, Cryptococcaceae, Cunninghamellaceae, Demetiaceae, Moniliaceae, Mortierellaceae, Mucoraceae, Pythiaceae, Sacharomycetaceae, Saprolegniaceae, Schizosacharomycetaceae, Sodariaceae or Tuberculariaceae. Further preferred micro-organisms are selected from the group: Choanephoraceae such as the genera Blakeslee, Choanephora, for example the genera and species Blakeslea trispora, Choanephora cucurbitarum, Choanephora infundibuliferavar. cucurbitarum, Mortierellaceae, such as the genus Mortierella, for example the genera and species Mortierella isabellina, Mortierella polycephala, Mortierella ramanniana, Mortierella vinacea, Mortierella zonata, Pythiaceae such as the genera Phytium, Phytophthora for example the genera and species Pythium debaryanum, Pythium intermedium, Pythium irregulare, Pythium megalacanthum, Pythium paroecandrum, Pythium sylvaticum, Pythium ultimum, Phytophthora cactorum, Phytophthora cinnamomi, Phytophthora citricola, Phytophthora citrophthora, Phytophthora cryptogea, Phytophthora drechsleri, Phytophthora erythroseptica, Phytophthora lateralis, Phytophthora megasperma, Phytophthora nicotianae, Phytophthora nicotianae var. parasitica, Phytophthora palmivora, Phytophthora parasitica, Phytophthora syringae, Saccharomycetaceae such as the genera Hansenula, Pichia, Saccharomyces, Saccharomycodes, Yarrowia for example the genera and species Hansenula anomala, Hansenula californica, Hansenula canadensis, Hansenula capsulata, Hansenula ciferrii, Hansenula glucozyma, Hansenula henricii, Hansenula holstii, Hansenula minuta, Hansenula nonfermentans, Hansenula philodendri, Hansenula polymorpha, Hansenula satumus, Hansenula subpelliculosa, Hansenula wickerhamii, Hansenula wingei, Pichia alcoholophila, Pichia angusta, Pichia anomala, Pichia bispora, Pichia burtonii, Pichia canadensis, Pichia capsulata, Pichia carsonii, Pichia cellobiosa, Pichia ciferrii, Pichia farinosa, Pichia fermentans, Pichia finlandica, Pichia glucozyma, Pichia guilliermondii, Pichia haplophila, Pichia henricii, Pichia holstii, Pichia jadinii, Pichia lindnerii, Pichia membranaefaciens, Pichia methanolica, Pichia minuta var. minuta, Pichia minuta var. nonfermentans, Pichia norvegensis, Pichia ohmeri, Pichia pastoris, Pichia philodendri, Pichia pini, Pichia polymorpha, Pichia quercuum, Pichia rhodanensis, Pichia sargentensis, Pichia stipitis, Pichia strasburgensis, Pichia subpelliculosa, Pichia toletana, Pichia trehalophila, Pichia vini, Pichia xylosa, Saccharomyces aceti, Saccharomyces bailii, Saccharomyces bayanus, Saccharomyces bisporus, Saccharomyces capensis, Saccharomyces carlsbergensis, Saccharomyces cerevisiae, Saccharomyces cerevisiae var. ellipsoideus, Saccharomyces chevalieri, Saccharomyces delbrueckii, Saccharomyces diastaticus, Saccharomyces drosophilarum, Saccharomyces elegans, Saccharomyces ellipsoideus, Saccharomyces fermentati, Saccharomyces florentinus, Saccharomyces fragilis, Saccharomyces heterogenicus, Saccharomyces hienipiensis, Saccharomyces inusitatus, Saccharomyces italicus, Saccharomyces kluyveri, Saccharomyces krusei, Saccharomyces lactis, Saccharomyces marxianus, Saccharomyces microellipsoides, Saccharomyces montanus, Saccharomyces norbensis, Saccharomyces oleaceus, Saccharomyces paradoxus, Saccharomyces pastorianus, Saccharomyces pretoriensis, Saccharomyces rosei, Saccharomyces rouxii, Saccharomyces uvarum, Saccharomycodes ludwigii, Yarrowia lipolytica, Schizosacharomycetaceae such as the genera Schizosaccharomyces e.g. the species Schizosaccharomyces japonicus var. japonicus, Schizosaccharomyces japonicus var. versatilis, Schizosaccharomyces malidevorans, Schizosaccharomyces octosporus, Schizosaccharomyces pombe var. malidevorans, Schizosaccharomyces pombe var. pombe, Thraustochytriaceae such as the genera Althomia, Aplanochytrium, Japonochytrium, Schizochytrium, Thraustochytrium e.g. the species Schizochytrium aggregatum, Schizochytrium limacinum, Schizochytrium mangrovei, Schizochytrium minutum, Schizochytrium octosporum, Thraustochytrium aggregatum, Thraustochytrium amoeboideum, Thraustochytrium antacticum, Thraustochytrium arudimentale, Thraustochytrium aureum, Thraustochytrium benthicola, Thraustochytrium globosum, Thraustochytrium indicum, Thraustochytrium kerguelense, Thraustochytrium kinnei, Thraustochytrium motivum, Thraustochytrium multirudimentale, Thraustochytrium pachydermum, Thraustochytrium proliferum, Thraustochytrium roseum, Thraustochytrium rossii, Thraustochytrium striatum or Thraustochytrium visurgense. Further preferred microorganisms are bacteria selected from the group of the families Bacillaceae, Enterobacteriacae or Rhizobiaceae. Examples of such micro-organisms may be selected from the group: Bacillaceae such as the genera Bacillus for example the genera and species Bacillus acidocaldarius, Bacillus acidoterrestris, Bacillus alcalophilus, Bacillus amyloliquefaciens, Bacillus amylolyticus, Bacillus brevis, Bacillus cereus, Bacillus circulans, Bacillus coagulans, Bacillus sphaericus subsp. fusiformis, Bacillus galactophilus, Bacillus globisporus, Bacillus globisporus subsp. marinus, Bacillus halophilus, Bacillus lentimorbus, Bacillus lentus, Bacillus licheniformis, Bacillus megaterium, Bacillus polymyxa, Bacillus psychrosaccharolyticus, Bacillus pumilus, Bacillus sphaericus, Bacillus subtilis subsp. spizizenii, Bacillus subtilis subsp. subtilis or Bacillus thuringiensis; Enterobacteriacae such as the genera Citrobacter, Edwardsiella, Enterobacter, Erwinia, Escherichia, Klebsiella, Salmonella or Serratia for example the genera and species Citrobacter amalonaticus, Citrobacter diversus, Citrobacter freundii, Citrobacter genomospecies, Citrobacter gillenii, Citrobacter intermedium, Citrobacter koseri, Citrobacter murliniae, Citrobacter sp., Edwardsiella hoshinae, Edwardsiella ictaluri, Edwardsiella tarda, Erwinia alni, Erwinia amylovora, Erwinia ananatis, Erwinia aphidicola, Erwinia billingiae, Erwinia cacticida, Erwinia cancerogena, Erwinia carnegieana, Erwinia carotovora subsp. atroseptica, Erwinia carotovora subsp. betavasculorum, Erwinia carotovora subsp. odorifera, Erwinia carotovora subsp. wasabiae, Erwinia chrysanthemi, Erwinia cypripedii, Erwinia dissolvens, Erwinia herbicola, Erwinia mallotivora, Erwinia milletiae, Erwinia nigrifluens, Erwinia nimipressuralis, Erwinia persicina, Erwinia psidii, Erwinia pyrifoliae, Erwinia quercina, Erwinia rhapontici, Erwinia rubrifaciens, Erwinia salicis, Erwinia stewartii, Erwinia tracheiphila, Erwinia uredovora, Escherichia adecarboxylata, Escherichia anindolica, Escherichia aurescens, Escherichia blattae, Escherichia coli, Escherichia coli var. communior, Escherichia coli-mutabile, Escherichia fergusonii, Escherichia hermannii, Escherichia sp., Escherichia vulneris, Klebsiella aerogenes, Klebsiella edwardsii subsp. atlantae, Klebsiella ornithinolytica, Klebsiella oxytoca, Klebsiella planticola, Klebsiella pneumoniae, Klebsiella pneumoniae subsp. pneumoniae, Klebsiella sp., Klebsiella terrigena, Klebsiella trevisanii, Salmonella abony, Salmonella arizonae, Salmonella bongori, Salmonella choleraesuis subsp. arizonae, Salmonella choleraesuis subsp. bongori, Salmonella choleraesuis subsp. cholereasuis, Salmonella choleraesuis subsp. diarizonae, Salmonella choleraesuis subsp. houtenae, Salmonella choleraesuis subsp. indica, Salmonella choleraesuis subsp. salamae, Salmonella daressalaam, Salmonella enterica subsp. houtenae, Salmonella enterica subsp. salamae, Salmonella enteritidis, Salmonella gallinarum, Salmonella heidelberg, Salmonella panama, Salmonella senftenberg, Salmonella typhimurium, Serratia entomophila, Serratia ficaria, Serratia fonticola, Serratia grimesii, Serratia liquefaciens, Serratia marcescens, Serratia marcescens subsp. marcescens, Serratia marinorubra, Serratia odorifera, Serratia plymouthensis, Serratia plymuthica, Serratia proteamaculans, Serratia proteamaculans subsp. quinovora, Serratia quinivorans or Serratia rubidaea; Rhizobiaceae such as the genera Agrobacterium, Carbophilus, Chelatobacter, Ensifer, Rhizobium, Sinorhizobium for example the genera and species Agrobacterium atlanticum, Agrobacterium ferrugineum, Agrobacterium gelatinovorum, Agrobacterium larrymoorei, Agrobacterium meteori, Agrobacterium radiobacter, Agrobacterium rhizogenes, Agrobacterium rubi, Agrobacterium stellulatum, Agrobacterium tumefaciens, Agrobacterium vitis, Carbophilus carboxidus, Chelatobacter heintzii, Ensifer adhaerens, Ensifer arboris, Ensifer fredii, Ensifer kostiensis, Ensifer kummerowiae, Ensifer medicae, Ensifer meliloti, Ensifer saheli, Ensifer terangae, Ensifer xinjiangensis, Rhizobium ciceri Rhizobium etli, Rhizobium fredii, Rhizobium galegae, Rhizobium gallicum, Rhizobium giardinii, Rhizobium hainanense, Rhizobium huakuii, Rhizobium huautlense, Rhizobium indigoferae, Rhizobium japonicum, Rhizobium leguminosarum, Rhizobium loessense, Rhizobium loti, Rhizobium lupini, Rhizobium mediterraneum, Rhizobium meliloti, Rhizobium mongolense, Rhizobium phaseoli, Rhizobium radiobacter, Rhizobium rhizogenes, Rhizobium rubi, Rhizobium sullae, Rhizobium tianshanense, Rhizobium trifolii, Rhizobium tropici, Rhizobium undicola, Rhizobium vitis, Sinorhizobium adhaerens, Sinorhizobium arboris, Sinorhizobium fredii, Sinorhizobium kostiense, Sinorhizobium kummerowiae, Sinorhizobium medicae, Sinorhizobium meliloti, Sinorhizobium morelense, Sinorhizobium saheli or Sinorhizobium xinjiangense.

How to culture the aforementioned micro-organisms is well known to the person skilled in the art.

The present invention also relates to a non-human transgenic organism, preferably a plant or seed thereof, comprising the polynucleotide or the vector of the present invention.

The term “non-human transgenic organism”, preferably, relates to a plant, a plant seed, an non-human animal or a multicellular micro-organism. The polynucleotide or vector may be present in the cytoplasm of the organism or may be incorporated into the genome either heterologous or by homologous recombination. Host cells, in particular those obtained from plants or animals, may be introduced into a developing embryo in order to obtain mosaic or chimeric organisms, i.e. non-human transgenic organisms comprising the host cells of the present invention. Suitable transgenic organisms are, preferably, all organisms which are suitable for the expression of recombinant genes.

Preferred plants to be used for making non-human transgenic organisms according to the present invention are all dicotyledonous or monocotyledonous plants, algae or mosses. Advantageous plants are selected from the group of the plant families Adelotheciaceae, Anacardiaceae, Asteraceae, Apiaceae, Betulaceae, Boraginaceae, Brassicaceae, Bromeliaceae, Caricaceae, Cannabaceae, Convolvulaceae, Chenopodiaceae, Crypthecodiniaceae, Cucurbitaceae, Ditrichaceae, Elaeagnaceae, Ericaceae, Euphorbiaceae, Fabaceae, Geraniaceae, Gramineae, Juglandaceae, Lauraceae, Leguminosae, Linaceae, Prasinophyceae or vegetable plants or ornamentals such as Tagetes. Examples which may be mentioned are the following plants selected from the group consisting of: Adelotheciaceae such as the genera Physcomitrella, such as the genus and species Physcomitrella patens, Anacardiaceae such as the genera Pistacia, Mangifera, Anacardium, for example the genus and species Pistacia vera [pistachio], Mangifer indica [mango] or Anacardium occidentale [cashew], Asteraceae, such as the genera Calendula, Carthamus, Centaurea, Cichorium, Cynara, Helianthus, Lactuca, Locusta, Tagetes, Valeriana, for example the genus and species Calendua officinalis [common marigold], Carthamus tinctorius [safflower], Centaurea cyanus [cornflower], Cichorium intybus [chicory], Cynara scolymus [artichoke], Helianthus annus [sunflower], Lactuca sativa, Lactuca crispa, Lactuca esculenta, Lactuca scariola L. ssp. sativa, Lactuca scariola L. var. integrate, Lactuca scariola L. var. integrifolia, Lactuca sativa subsp. romana, Locusta communis, Valeriana locusta [salad vegetables], Tagetes lucida, Tagetes erecta or Tagetes tenuifolia [african or french marigold], Apiaceae, such as the genus Daucus, for example the genus and species Daucus carota [carrot], Betulaceae, such as the genus Corylus, for example the genera and species Corylus avellana or Corylus colurna [hazelnut], Boraginaceae, such as the genus Borago, for example the genus and species Borago officinalis [borage], Brassicaceae, such as the genera Brassica, Melanosinapis, Sinapis, Arabadopsis, for example the genera and species Brassica napus, Brassica rapa ssp. [oilseed rape], Sinapis arvensis Brassica juncea, Brassica juncea var. juncea, Brassica juncea var. crispifolia, Brassica juncea var. foliosa, Brassica nigra, Brassica sinapioides, Melanosinapis communis [mustard], Brassica oleracea [fodder beet] or Arabidopsis thaliana, Bromeliaceae, such as the genera Anana, Bromelia (pineapple), for example the genera and species Anana comosus, Ananas ananas or Bromelia comosa [pineapple], Caricaceae, such as the genus Carica, such as the genus and species Carica papaya [pawpaw], Cannabaceae, such as the genus Cannabis, such as the genus and species Cannabis sativa [hemp], Convolvulaceae, such as the genera Ipomea, Convolvulus, for example the genera and species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, batate], Chenopodiaceae, such as the genus Beta, such as the genera and species Beta vulgaris, Beta vulgaris var. altissima, Beta vulgaris var. Vulgaris, Beta maritima, Beta vulgaris var. perennis, Beta vulgaris var. conditiva or Beta vulgaris var. esculenta [sugarbeet], Crypthecodiniaceae, such as the genus Crypthecodinium, for example the genus and species Cryptecodinium cohnii, Cucurbitaceae, such as the genus Cucurbita, for example the genera and species Cucurbita maxima, Cucurbita mixta, Cucurbita pepo or Cucurbita moschata [pumpkin/squash], Cymbellaceae such as the genera Amphora, Cymbella, Okedenia, Phaeodactylum, Reimeria, for example the genus and species Phaeodactylum tricornutum, Ditrichaceae such as the genera Ditrichaceae, Astomiopsis, Ceratodon, Chrysoblastella, Ditrichum, Distichium, Eccremidium, Lophidion, Philibertiella, Pleuridium, Saelania, Trichodon, Skottsbergia, for example the genera and species Ceratodon antarcticus, Ceratodon columbiae, Ceratodon heterophyllus, Ceratodon purpureus, Ceratodon purpureus, Ceratodon purpureus ssp. convolutus, Ceratodon, purpureus spp. stenocarpus, Ceratodon purpureus var. rotundifolius, Ceratodon ratodon, Ceratodon stenocarpus, Chrysoblastella chilensis, Ditrichum ambiguum, Ditrichum brevisetum, Ditrichum crispatissimum, Ditrichum difficile, Ditrichum falcifolium, Ditrichum flexicaule, Ditrichum giganteum, Ditrichum heteromallum, Ditrichum lineare, Ditrichum lineare, Ditrichum montanum, Ditrichum montanum, Ditrichum pallidum, Ditrichum punctulatum, Ditrichum pusillum, Ditrichum pusillum var. tortile, Ditrichum rhynchostegium, Ditrichum schimperi, Ditrichum tortile, Distichium capillaceum, Distichium hagenii, Distichium inclinatum, Distichium macounii, Eccremidium floridanum, Eccremidium whiteleggei, Lophidion strictus, Pleuridium acuminatum, Pleuridium alternifolium, Pleuridium holdridgei, Pleuridium mexicanum, Pleuridium ravenelii, Pleuridium subulatum, Saelania glaucescens, Trichodon borealis, Trichodon cylindricus or Trichodon cylindricus var. oblongus, Elaeagnaceae such as the genus Elaeagnus, for example the genus and species Olea europaea [olive], Ericaceae such as the genus Kalmia, for example the genera and species Kalmia latifolia, Kalmia angustifolia, Kalmia microphylla, Kalmia polifolia, Kalmia occidentalis, Cistus chamaerhodendros or Kalmia lucida [mountain laurel], Euphorbiaceae such as the genera Manihot, Janipha, Jatropha, Ricinus, for example the genera and species Manihot utilissima, Janipha manihot, Jatropha manihot, Manihot aipil, Manihot dulcis, Manihot manihot, Manihot melanobasis, Manihot esculenta [manihot] or Ricinus communis [castor-oil plant], Fabaceae such as the genera Pisum, Albizia, Cathormion, Feuillea, Inga, Pithecolobium, Acacia, Mimosa, Medicajo, Glycine, Dolichos, Phaseolus, Soja, for example the genera and species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea julibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [silk tree], Medicago sativa, Medicago falcata, Medicago varia [alfalfa], Glycine max Dolichos soja, Glycine gracilis, Glycine hispida, Phaseolus max, Soja hispida or Soja max [soybean], Funariaceae such as the genera Aphanorrhegma, Entosthodon, Funaria, Physcomitrella, Physcomitrium, for example the genera and species Aphanorrhegma serratum, Entosthodon attenuatus, Entosthodon bolanderi, Entosthodon bonplandii, Entosthodon californicus, Entosthodon drummondii, Entosthodon jamesonii, Entosthodon leibergii, Entosthodon neoscoticus, Entosthodon rubrisetus, Entosthodon spathulifolius, Entosthodon tucsoni, Funaria americana, Funaria bolanderi, Funaria calcarea, Funaria californica, Funaria calvescens, Funaria convoluta, Funaria flavicans, Funaria groutiana, Funaria hygrometrica, Funaria hygrometrica var. arctica, Funaria hygrometrica var. calvescens, Funaria hygrometrica var. convoluta, Funaria hygrometrica var. muralis, Funaria hygrometrica var. utahensis, Funaria microstoma, Funaria microstoma var. obtusifolia, Funaria muhlenbergii, Funaria orcuttii, Funaria plano-convexa, Funaria polaris, Funaria ravenelii, Funaria rubriseta, Funaria serrata, Funaria sonorae, Funaria sublimbatus, Funaria tucsoni, Physcomitrella californica, Physcomitrella patens, Physcomitrella readeri, Physcomitrium australe, Physcomitrium californicum, Physcomitrium collenchymatum, Physcomitrium coloradense, Physcomitrium cupuliferum, Physcomitrium drummondii, Physcomitrium eurystomum, Physcomitrium flexifolium, Physcomitrium hookeri, Physcomitrium hookeri var. serratum, Physcomitrium immersum, Physcomitrium kellermanii, Physcomitrium megalocarpum, Physcomitrium pyriforme, Physcomitrium pyriforme var. serratum, Physcomitrium rufipes, Physcomitrium sandbergii, Physcomitrium subsphaericum, Physcomitrium washingtoniense, Geraniaceae, such as the genera Pelargonium, Cocos, Oleum, for example the genera and species Cocos nucifera, Pelargonium grossularioides or Oleum cocois [coconut], Gramineae, such as the genus Saccharum, for example the genus and species Saccharum officinarum, Juglandaceae, such as the genera Juglans, Wallia, for example the genera and species Juglans regia, Juglans ailanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut], Lauraceae, such as the genera Persea, Laurus, for example the genera and species Laurus nobilis [bay], Persea americana, Persea gratissima or Persea persea [avocado], Leguminosae, such as the genus Arachis, for example the genus and species Arachis hypogaea [peanut], Linaceae, such as the genera Linum, Adenolinum, for example the genera and species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [linseed], Lythrarieae, such as the genus Punica, for example the genus and species Punica granatum [pomegranate], Malvaceae, such as the genus Gossypium, for example the genera and species Gossypium hirsutum, Gossypium arboreum, Gossypium barbadense, Gossypium herbaceum or Gossypium thurberi [cotton], Marchantiaceae, such as the genus Marchantia, for example the genera and species Marchantia berteroana, Marchantia foliacea, Marchantia macropora, Musaceae, such as the genus Musa, for example the genera and species Musa nana, Musa acuminata, Musa paradisiaca, Musa spp. [banana], Onagraceae, such as the genera Camissonia, Oenothera, for example the genera and species Oenothera biennis or Camissonia brevipes [evening primrose], Palmae, such as the genus Elacis, for example the genus and species Elaeis guineensis [oil palm], Papaveraceae, such as the genus Papaver, for example the genera and species Papaver orientale, Papaver rhoeas, Papaver dubium [poppy], Pedaliaceae, such as the genus Sesamum, for example the genus and species Sesamum indicum [sesame], Piperaceae, such as the genera Piper, Artanthe, Peperomia, Steffensia, for example the genera and species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata [cayenne pepper], Poaceae, such as the genera Hordeum, Secale, Avena, Sorghum, Andropogon, Holcus, Panicum, Oryza, Zea (maize), Triticum, for example the genera and species Hordeum vulgare, Hordeum jubatum, Hordeum murinum, Hordeum secalinum, Hordeum distichon, Hordeum aegiceras, Hordeum hexastichon, Hordeum hexastichum, Hordeum irregulare, Hordeum sativum, Hordeum secalinum [barley], Secale cereale [rye], Avena sativa, Avena fatua, Avena byzantina, Avena fatua var. sativa, Avena hybrida [oats], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum, Panicum militaceum [millet], Oryza sativa, Oryza latifolia [rice], Zea mays [maize], Triticum aestivum, Triticum durum, Triticum turgidum, Triticum hybernum, Triticum macha, Triticum sativum or Triticum vulgare [wheat], Porphyridiaceae, such as the genera Chroothece, Flintiella, Petrovanella, Porphyridium, Rhodella, Rhodosorus, Vanhoeffenia, for example the genus and species Porphyridium cruentum, Proteaceae, such as the genus Macadamia, for example the genus and species Macadamia intergrifolia [macadamia], Prasinophyceae such as the genera Nephroselmis, Prasinococcus, Scherffelia, Tetraselmis, Mantoniella, Ostreococcus, for example the genera and species Nephroselmis olivacea, Prasinococcus capsulatus, Scherffelia dubia, Tetraselmis chui, Tetraselmis suecica, Mantoniella squamata, Ostreococcus tauri, Rubiaceae such as the genus Cofea, for example the genera and species Cofea spp., Coffea arabica, Coffea canephora or Coffea liberica [coffee], Scrophulariaceae such as the genus Verbascum, for example the genera and species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein], Solanaceae such as the genera Capsicum, Nicotiana, Solanum, Lycopersicon, for example the genera and species Capsicum annuum, Capsicum annuum var. glabriusculum, Capsicum frutescens [pepper], Capsicum annuum [paprika], Nicotiana tabacum, Nicotiana alata, Nicotiana attenuate, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [eggplant], Lycopersicon esculentum, Lycopersicon lycopersicum, Lycopersicon pyriforme, Solanum integrifolium or Solanum lycopersicum [tomato], Sterculiaceae, such as the genus Theobroma, for example the genus and species Theobroma cacao [cacao] or Theaceae, such as the genus Camellia, for example the genus and species Camellia sinensis [tea]. In particular preferred plants to be used as transgenic plants in accordance with the present invention are oil fruit crops which comprise large amounts of lipid compounds, such as peanut, oilseed rape, canola, sunflower, safflower, poppy, mustard, hemp, castor-oil plant, olive, sesame, Calendula, Punica, evening primrose, mullein, thistle, wild roses, hazelnut, almond, macadamia, avocado, bay, pumpkin/squash, linseed, soybean, pistachios, borage, trees (oil palm, coconut, walnut) or crops such as maize, wheat, rye, oats, triticale, rice, barley, cotton, cassava, pepper, Tagetes, Solanaceae plants such as potato, tobacco, eggplant and tomato, Vicia species, pea, alfalfa or bushy plants (coffee, cacao, tea), Salix species, and perennial grasses and fodder crops. Preferred plants according to the invention are oil crop plants such as peanut, oilseed rape, canola, sunflower, safflower, poppy, mustard, hemp, castor-oil plant, olive, Calendula, Punica, evening primrose, pumpkin/squash, linseed, soybean, borage, trees (oil palm, coconut). Especially preferred are plants which are high in C18:2- and/or C18:3-fatty acids, such as sunflower, safflower, tobacco, mullein, sesame, cotton, pumpkin/squash, poppy, evening primrose, walnut, linseed, hemp, thistle or safflower. Very especially preferred plants are plants such as safflower, sunflower, poppy, evening primrose, walnut, linseed, or hemp.

Preferred mosses are Physcomitrella or Ceratodon. Preferred algae are Isochrysis, Mantoniella, Ostreococcus or Crypthecodinium, and algae/diatoms such as Phaeodactylum or Thraustochytrium. More preferably, said algae or mosses are selected from the group consisting of: Shewanella, Physcomitrella, Thraustochytrium, Fusarium, Phytophthora, Ceratodon, Isochrysis, Aleurita, Muscarioides, Mortierella, Phaeodactylum, Cryphthecodinium, specifically from the genera and species Thallasiosira pseudonona, Euglena gracilis, Physcomitrella patens, Phytophtora infestans, Fusarium graminaeum, Cryptocodinium cohnii, Ceratodon purpureus, Isochrysis galbana, Aleurita farinosa, Thraustochytrium sp., Muscarioides viallii, Mortierella alpina, Phaeodactylum tricornutum or Caenorhabditis elegans or especially advantageously Phytophtora infestans, Thallasiosira pseudonona and Cryptocodinium cohnii.

Transgenic plants may be obtained by transformation techniques as published, and cited, in: Plant Molecular Biology and Biotechnology (CRC Press, Boca Raton, Fla.), chapter 6/7, pp.71-119 (1993); F. F. White, Vectors for Gene Transfer in Higher Plants; in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus, Annu. Rev. Plant Physiol. Plant Molec. Biol. 42 (1991), 205-225. Preferably, transgenic plants can be obtained by T-DNA-mediated transformation. Such vector systems are, as a rule, characterized in that they contain at least the vir genes, which are required for the Agrobacterium-mediated transformation, and the sequences which delimit the T-DNA (T-DNA border). Suitable vectors are described elsewhere in the specification in detail.

Preferably, a multicellular micro-organism as used herein refers to protists or diatoms. More preferably, it is selected from the group of the families Dinophyceae, Turaniellidae or Oxytrichidae, such as the genera and species: Crypthecodinium cohnii, Phaeodactylum tricornutum, Stylonychia mytilus, Stylonychia pustulata, Stylonychia putrina, Stylonychia notophora, Stylonychia sp., Colpidium campylum or Colpidium sp.

The present invention also relates to a method for expressing a nucleic acid of interest in a host cell comprising
(a) introducing the polynucleotide or the vector of the present invention into the host cell, whereby the nucleic acid sequence of interest will be operatively linked to the expression control sequence; and
(b) expressing the said nucleic acid sequence in said host cell.

The polynucleotide or vector of the present invention can be introduced into the host cell by suitable transfection or transformation techniques as specified elsewhere in this description. The nucleic acid of interest will be expressed in the host cell under suitable conditions. To this end, the host cell will be cultivated under conditions which, in principle, allow for transcription of nucleic acids. Moreover, the host cell, preferably, comprises the exogenously supplied or endogenously present transcription machinery required for expressing a nucleic acid of interest by the expression control sequence. More preferably, the host cell is a plant cell and, most preferably, a seed cell or precursor thereof.

Moreover, the present invention encompasses a method for expressing a nucleic acid of interest in a non-human organism comprising
(a) introducing the polynucleotide or the vector of the present invention into the non human organism, whereby the nucleic acid sequence of interest will be operatively linked to the expression control sequence; and
(b) expressing the said nucleic acid sequence in said non-human transgenic organism.

The polynucleotide or vector of the present invention can be introduced into the non-human transgenic organism by suitable techniques as specified elsewhere in this description. The non-human transgenic organism, preferably, comprises the exogenously supplied or endogenously present transcription machinery required for expressing a nucleic acid of interest by the expression control sequence. More preferably, the non-human transgenic organism is a plant or seed thereof. It is to be understood that the nucleic acid of interest will be expressed, preferably, seed specific in the said non-human transgenic organism.

In the following tables 1 to 6, the cis-regulatory elements found in the expression control sequences of the present invention are shown.

TABLE 1
cis-regulatory elements of SEQ ID NO: 7

| | | | Opt. | | | | | | |
|---|---|---|---|---|---|---|---|---|---|
| Seq. | Family/ | | thres | Start | End | | Core | Matrix | |
| name | matrix | Further Information | h. | pos. | pos. | Strand | sim. | sim. | Sequence |
| SEQ_7 | P$TBPF/ | Plant TATA box | 0.90 | 4 | 18 | + | 1.000 | 0.930 | gtcaTATAta |
| | TATA.02 | | | | | | | | tatga |
| SEQ_7 | P$TBPF/ | Plant TATA box | 0.90 | 5 | 19 | − | 1.000 | 0.930 | gtcaTATAta |
| | TATA.02 | | | | | | | | tatga |
| SEQ_7 | P$PSRE/ | GAAA motif involved in pollen specific | 0.83 | 20 | 36 | − | 1.000 | 0.886 | caaaaGAAAc |
| | GAAA.01 | transcriptional activation | | | | | | | tatggaa |
| SEQ_7 | P$GTBX/ | SBF-1 | 0.87 | 33 | 49 | + | 1.000 | 0.087 | tttggagTTA |
| | SBF1.01 | | | | | | | | Aacgcat |
| SEQ_7 | P$NACF/ | Wheat NACdomain DNA binding factor | 0.68 | 60 | 82 | + | 1.000 | 0.680 | tatgatttag |
| | TANAC69.01 | | | | | | | | cTACGtgaca |
| | | | | | | | | | gaa |
| SEQ_7 | P$GBOX/ | Oryza sativa bZIP protein 8 | 0.84 | 64 | 84 | + | 1.000 | 0.899 | atttagctAC |
| | OSBZ8.01 | | | | | | | | GTgacaggaa |
| | | | | | | | | | aa |
| SEQ_7 | P$ABRE/ | ABA response elements | 0.82 | 65 | 81 | + | 1.000 | 0.853 | tttagctACG |
| | ABRE.01 | | | | | | | | Tgacaga |
| SEQ_7 | P$OPAQ/ | Rice transcription activator-1 (RITA), | 0.95 | 65 | 81 | − | 1.000 | 0.981 | tctgtcACGT |
| | RITA1.01 | basic leucin zipper protein, highly | | | | | | | agctaaa |
| | | expresses during seed development | | | | | | | |
| SEQ_7 | P$OPAQ/ | Rice transcription activator-1 (RITA), | 0.95 | 66 | 82 | + | 1.000 | 0.985 | ttagctACGT |
| | RITA1.01 | basic leucin zipper protein, highly | | | | | | | gacagaa |
| | | expresses during seed development | | | | | | | |
| SEQ_7 | P$AREF/ | Silencing element binding factor- | 0.96 | 70 | 82 | − | 1.000 | 0.964 | ttcTGTCacg |
| | SEBF.01 | transcriptional repressor | | | | | | | tag |
| SEQ_7 | P$TALE/ | Homeodomain protein of the Knotted class 1 | 1.00 | 73 | 85 | + | 1.000 | 1.000 | cgTGACagaa |
| | HVH21.01 | | | | | | | | aat |
| SEQ_7 | O$RPOA/ | Avian C-type LTR PolyA signal | 0.71 | 88 | 108 | + | 0.750 | 0.379 | cagatCAAAg |
| | APOLYA.01 | | | | | | | | tgtcgttttt |
| | | | | | | | | | t |
| SEQ_7 | 0$RPOA/ | Avian C-type LTR PolyA signal | 0.71 | 88 | 108 | + | 0.750 | 0.739 | tataaAAAAc |
| | APOLYA.01 | | | | | | | | gacactttga |
| | | | | | | | | | t |
| SEQ_7 | P$NACF/ | Wheat NACdomain DNA binding factor | 0.68 | 93 | 115 | − | 0.812 | 0.717 | ggtctataaa |
| | TANAC69.01 | | | | | | | | aAACGacact |
| | | | | | | | | | ttg |
| SEQ_7 | P$TBPF/ | Plant TATA box | 0.90 | 101 | 115 | − | 1.000 | 0.968 | ggtcTATAaa |
| | TATA.02 | | | | | | | | aaacg |
| SEQ_7 | P$AHBP/ | Homeodomain protein WUSCHEL | 0.94 | 121 | 131 | − | 1.000 | 1.000 | ttaatTAATg |
| | WUS.01 | | | | | | | | t |
| SEQ_7 | P$DOFF/ | Dof1/MNB1a-single zinc finger transcription | 0.98 | 122 | 138 | + | 1.000 | 0.980 | cattaattAA |
| | DOF1.01 | factor | | | | | | | AGgataa |
| SEQ_7 | P$MYBS/ | MybSt1 (Myb Solanum tuberosum 1) with a | 0.90 | 126 | 142 | − | 1.000 | 0.983 | tactttATCC |
| | MYBST1.01 | single myb repeat | | | | | | | tttaatt |
| SEQ_7 | P$1BOX/ | Class I GATA factors | 0.93 | 129 | 145 | + | 1.000 | 0.967 | taaagGATAa |
| | GATA.01 | | | | | | | | agtaaga |
| SEQ_7 | P$NCS1/ | Nodulin consensus sequence 1 | 0.85 | 129 | 139 | + | 0.804 | 0.884 | tAAAGgataa |
| | NCS1.01 | | | | | | | | a |
| SEQ_7 | P$MYCL/ | ICE (inducer of CBF expression 1), | 0.95 | 161 | 179 | + | 0.954 | 0.966 | aggaaACAAa |
| | ICE.01 | AtMYC2(rd22BP1) | | | | | | | tgatttcca |
| SEQ_7 | P$PSRE/ | GAAA motif involved in pollen specific | 0.83 | 166 | 182 | − | 1.000 | 0.842 | ttgtgGAAAt |
| | GAAA.01 | transcriptional activation | | | | | | | catttgt |
| SEQ_7 | P$AHBP/ | Arabidopsis thaliana homeo box protein 1 | 0.90 | 167 | 177 | + | 0.789 | 0.900 | caaATGAttt |
| | ATHB1.01 | | | | | | | | c |
| SEQ_7 | P$AHBP/ | HDZip class I protein ATHB5 | 0.89 | 167 | 177 | − | 0.936 | 0.936 | gaaATCAttt |
| | ATHB5.01 | | | | | | | | g |
| SEQ_7 | P$NCS1/ | Nodulin consensus sequence 1 | 0.85 | 167 | 177 | + | 0.887 | 0.904 | cAAATgattt |
| | NCS1.01 | | | | | | | | c |
| SEQ_7 | P$MYBL/ | CAACTC regulatory elements, GA-inducible | 0.83 | 192 | 208 | − | 1.000 | 0.836 | attcgaaAGT |
| | CARE.01 | | | | | | | | Tgattca |
| SEQ_7 | P$DOFF/ | Dof1/MNB1a-single zinc finger transcription | 0.98 | 205 | 221 | − | 1.000 | 0.987 | accaaattAA |
| | DOF1.01 | factor | | | | | | | AGtattc |
| SEQ_7 | P$WBXF/ | Elicitor response element | 0.89 | 213 | 229 | − | 1.000 | 0.918 | tgagctTGAC |
| | ERE.01 | | | | | | | | caaatta |
| SEQ_7 | O$RVUP/ | Upstream element of C-type Long Terminal | 0.76 | 227 | 247 | − | 1.000 | 0.783 | actcacatga |
| | LTRUP.01 | Repeats | | | | | | | gTTTCgtgtg |
| | | | | | | | | | a |
| SEQ_7 | P$LEGB/ | Legumin box, highly conserved sequence | 0.59 | 263 | 289 | − | 0.750 | 0.601 | tacatatCCA |
| | LEGB.01 | element about 100 by upstream of the TSS | | | | | | | Aatcagaggt |
| | | in legumin genes | | | | | | | agaaggc |
| SEQ_7 | P$MYBS/ | Rice MYB proteins with single DNA binding | 0.82 | 274 | 290 | − | 1.000 | 0.850 | gtacaTATCc |
| | OSMYBS.01 | domains, binding to the amylase element | | | | | | | aaatcag |
| | | (TATCCA) | | | | | | | |
| SEQ_7 | P$GTBX/ | SBF-1 | 0.87 | 284 | 300 | + | 1.000 | 0.789 | tatgtacTTA |
| | SBF1.01 | | | | | | | | Aaacact |
| SEQ_7 | P$EINL/ | TEIL (tobacco EIN3-like) | 0.92 | 285 | 293 | + | 1.000 | 0.935 | aTGTActta |
| | TEIL.01 | | | | | | | | |
| | | | | | | | | | acttaaaaca |
| SEQ_7 | O$RVUP/ | Upstream element of C-type Long Terminal | 0.76 | 289 | 309 | + | 1.000 | 0.784 | cTTTCtgagg |
| | OTRUP.01 | Repeats | | | | | | | aa |
| SEQ_7 | P$TELO/ | Arabidopsis Telo-box interacting protein | 0.85 | 308 | 322 | + | 1.000 | 0.896 | aaacACCCta |
| | ATPURA.01 | releated to the conserved animal protein | | | | | | | acgct |
| | | Pur-alpha | | | | | | | |
| SEQ_7 | P$MIIG/ | Maize activator P of flavonoid biosynthetic | 0.93 | 320 | 334 | + | 0.966 | 0.973 | gcttGGTTgg |
| | P_ACT.01 | genes | | | | | | | tggat |
| SEQ_7 | P$MYBL/ | Myb-like protein of Petunia hybrida | 0.80 | 339 | 355 | − | 1.000 | 0.807 | ctaaaattGT |
| | MYBPH3.01 | | | | | | | | TAtgatg |
| SEQ_7 | O$RPOA/ | PolyA signal of D-type LTRs | 0.78 | 348 | 368 | − | 0.750 | 0.834 | aCCAAtaaaa |
| | DTYPEPA.01 | | | | | | | | aatctaaaat |
| | | | | | | | | | t |
| SEQ_7 | P$LREM/ | Motif involved in carotenoid and toco- | 0.85 | 349 | 359 | − | 1.000 | 0.990 | aaATCTaaaa |
| | ATCTA.01 | pherol biosynthesis and in the expression | | | | | | | t |
| | | of photosynthesis-related genes | | | | | | | |
| SEQ_7 | P$CCAF/ | Circadian clock associated 1 | 0.85 | 352 | 366 | − | 1.000 | 0.971 | caataaaaAA |
| | CCA1.01 | | | | | | | | TCtaa |
| SEQ_7 | P$CAAT/ | CCAAT-box in plant promoters | 0.97 | 361 | 369 | − | 1.000 | 0.981 | caCCAAtaa |
| | CAAT.01 | | | | | | | | |
| SEQ_7 | P$OPAQ/ | Opaque-2 regulatory protein | 0.87 | 363 | 379 | − | 0.794 | 0.871 | aaccacataT |
| | O2.01 | | | | | | | | CACcaat |
| SEQ_7 | O$RPAD/ | Mammalian C-type LTR Poly A downstream | 0.87 | 371 | 383 | + | 1.000 | 0.874 | tatGTGGttt |
| | PADS.01 | element | | | | | | | tgt |
| SEQ_7 | P$MIIG/ | Putative cis-acting element in various PAL | 0.81 | 380 | 394 | + | 0.963 | 0.859 | ttGTGGttgg |
| | PALBOXP.01 | and 4CL gene promoters | | | | | | | agaag |
| SEQ_7 | O$RPOA/ | Lentiviral Poly A signal | 0.94 | 398 | 418 | − | 1.000 | 0.982 | tgaAATAaag |
| | LPOLYA.01 | | | | | | | | ttattagaac |
| | | | | | | | | | t |
| SEQ_7 | P$HMGF/ | High mobility group WY-like proteins | 0.89 | 408 | 422 | + | 1.000 | 0.895 | acttTATTtc |
| | HMG_IY.01 | | | | | | | | atcat |
| SEQ_7 | P$AHBP/ | Sunflower homeodomain leucine-zipper | 0.87 | 415 | 425 | + | 1.000 | 0.940 | ttcatcATTA |
| | HAHB4.01 | protein Hahb-4 | | | | | | | t |
| SEQ_7 | P$SBPD/ | SQUA promoter binding proteins | 0.88 | 435 | 451 | − | 1.000 | 0.902 | tactgGTACa |
| | SBP.01 | | | | | | | | atacagt |
| SEQ_7 | P$GTBX/ | Trihelix DNA-binding factor GT-3a | 0.83 | 437 | 453 | − | 0.750 | 0.839 | tatactGGTA |
| | GT3A.01 | | | | | | | | caataca |
| SEQ_7 | P$MADS/ | AGL1, Arabidopsis MADS-domain protein | 0.84 | 440 | 460 | − | 1.000 | 0.890 | gaaTGCCtat |
| | AGL1.01 | AGAMOUS-like 1 | | | | | | | actggtacaa |
| | | | | | | | | | t |
| SEQ_7 | P$MADS/ | AGL1, Arabidopsis MADS-domain protein | 0.84 | 441 | 461 | + | 0.995 | 0.862 | ttgTACCagt |
| | AGL1.01 | AGAMOUS-like 1 | | | | | | | ataggcattc |
| | | | | | | | | | t |
| SEQ_7 | P$1BOX/ | Class I GATA factors | 0.93 | 461 | 477 | + | 1.000 | 0.937 | tcttcGATAa |
| | GATA.01 | | | | | | | | tacaaat |
| SEQ_7 | P$WBXF/ | WRKY plant specific zinc-finger-type | 0.92 | 479 | 495 | − | 1.000 | 0.971 | aagttTTGAc |
| | WRKY.01 | factor associated with pathogen defence, | | | | | | | tattata |
| | | W box | | | | | | | |
| SEQ_7 | P$1BOX/ | Class I GATA factors | 0.93 | 495 | 511 | − | 1.000 | 0.946 | tatatGATAa |
| | GATA.01 | | | | | | | | ttagcaa |
| SEQ_7 | P$AHBP/ | Sunflower homeodomain leucine-zipper | 0.87 | 498 | 508 | − | 1.000 | 0.910 | atgataATTA |
| | HAHB4.01 | protein Hahb-4 | | | | | | | g |
| SEQ_7 | P$NCS1/ | Nodulin consensus sequence 1 | 0.85 | 516 | 526 | − | 0.878 | 0.868 | cAAATgatgt |
| | NCS1.01 | | | | | | | | g |
| SEQ_7 | P$L1BX/ | L1-specific homeodomain protein ATML1 | 0.82 | 528 | 544 | − | 1.000 | 0.823 | taagtaTAAA |
| | ATML1.01 | (A. thaliana meristem layer 1) | | | | | | | agtattg |
| SEQ_7 | P$SPF1/ | DNA-binding protein of sweet potato that | 0.87 | 529 | 541 | + | 1.000 | 0.923 | aaTACTttta |
| | SP8BF.01 | binds to the SP8a (ACTGTGTA)and SP8b | | | | | | | tac |
| | | (TACTATT) sequences of sporamin and beta- | | | | | | | |
| | | amylase genes | | | | | | | |
| SEQ_7 | P$STKM/ | Storekeeper (STK), plant specific DNA binding | 0.85 | 570 | 584 | − | 1.000 | 0.852 | accTAAAtaa |
| | STK.01 | protein important for tuber-specific and | | | | | | | tcaaa |
| | | sucrose-inducible gene expression | | | | | | | |
| SEQ_7 | P$GTBX/ | SBF-1 | 0.87 | 590 | 606 | + | 1.000 | 0.872 | ctcatttTTA |
| | SBF1.01 | | | | | | | | Atataga |
| SEQ_7 | P$SEF4/ | Soybean embryo factor 4 | 0.98 | 592 | 602 | + | 1.000 | 0.983 | caTTTTtaat |
| | SEF4.01 | | | | | | | | a |
| SEQ_7 | P$PSRE/ | GAAA motif involved in pollen specific | 0.83 | 600 | 616 | − | 1.000 | 0.872 | gtttaGAAAt |
| | GAAA.01 | transcriptional activation | | | | | | | tctatat |
| SEQ_7 | P$MYBL/ | Myb-like protein of Petunia hybrida | 0.80 | 608 | 624 | − | 0.750 | 0.845 | aaaaaacgGT |
| | MYBPH3.01 | | | | | | | | TTagaaa |
| SEQ_7 | P$MYBL/ | Myb-like protein of Petunia hybrida | 0.80 | 610 | 626 | + | 0.750 | 0.803 | tctaaaccGT |
| | MYBPH3.01 | | | | | | | | TTttttt |
| SEQ_7 | P$MSAE/ | M-phase-specific activators (NtmybA1, | 0.80 | 611 | 625 | − | 1.000 | 0.867 | aaaaaAACGg |
| | MSA.01 | NtmybA2, NtmybB) | | | | | | | tttag |
| SEQ_7 | P$DOFF/ | Prolamin box, conserved in cereal seed | 0.75 | 619 | 625 | − | 0.761 | 0.802 | tgaaatgaAA |
| | PBOX.01 | storage protein gene promoters | | | | | | | AAaaaaa |
| SEQ_7 | P$GAPB/ | Cis-element in the GAPDH promoters conferring | 0.88 | 621 | 635 | − | 1.000 | 0.960 | tgaaATGAaa |
| | GAP.01 | light inducibility | | | | | | | aaaaa |
| SEQ_7 | P$OPAQ/ | Opaque-2 regulatory protein | 0.87 | 624 | 640 | + | 1.000 | 0.924 | tttttcattT |
| | O2.01 | | | | | | | | CATcatc |
| SEQ_7 | P$DOFF/ | Prolamin box, conserved in cereal seed storage | 0.75 | 635 | 651 | − | 1.000 | 0.758 | tggttagaAA |
| | PBOX.01 | protein gene promoters | | | | | | | AGatgat |
| SEQ_7 | P$NCS1/ | Nodulin consensus sequence 1 | 0.85 | 635 | 645 | − | 1.000 | 0.963 | gAAAAgatga |
| | NCS1.01 | | | | | | | | t |
| SEQ_7 | P$DOFF/ | Prolamin box, conserved in cereal seed storage | 0.75 | 652 | 668 | − | 1.000 | 0.819 | gagactgtAA |
| | PBOX.01 | protein gene promoters | | | | | | | AGatgaa |
| SEQ_7 | P$AHBP/ | Sunflower homeodomain leucine-zipper protein | 0.87 | 675 | 685 | + | 1.000 | 0.892 | tatatgATTA |
| | HAHB4.01 | Hahb-4 | | | | | | | g |
| SEQ_7 | P$HMGF/ | High mobility group WY-like proteins | 0.89 | 684 | 698 | + | 1.000 | 0.905 | agttTATTtc |
| | HMG_IY.01 | | | | | | | | attcg |
| SEQ_7 | P$TBPF/ | Plant TATA box | 0.90 | 707 | 721 | + | 1.000 | 0.91 | tttcTATAta |
| | TATA.02 | | | | | | | | ttaaa |
| SEQ_7 | P$AHBP/ | Sunflower homeodomain leucine-zipper protein | 0.87 | 730 | 740 | − | 1.000 | 0.936 | tcgattATTA |
| | HAHB4.01 | Hahb-4 | | | | | | | t |
| SEQ_7 | P$MSAE/ | M-phase-specific activators (NtmybA1, | 0.80 | 746 | 760 | − | 0.750 | 0.803 | cttcaAATGg |
| | MSA.01 | NtmybA2, NtmybB) | | | | | | | tgata |
| SEQ_7 | P$MYCL/ | ICE (inducer of CBF expression 1), AtMYC2 | 0.95 | 770 | 788 | + | 1.000 | 0.953 | ttgtgACATt |
| | ICE.01 | (rd22BP1) | | | | | | | tggcattac |
| SEQ_7 | P$OCSE/ | bZIP transcription factor binding to | 0.73 | 773 | 793 | + | 0.974 | 0.811 | tgacatttgg |
| | OCSTF.01 | OCS-elements | | | | | | | catTACGgga |
| | | | | | | | | | a |
| SEQ_7 | P$MADS/ | Binding sites for AP1, AP3-PI and AG dimers | 0.75 | 794 | 814 | + | 1.000 | 0.765 | aggtcCCATg |
| | MADS.01 | | | | | | | | tatctcaaac |
| | | | | | | | | | t |
| SEQ_7 | P$EINL/ | TEIL (tobacco EIN3-like) | 0.92 | 801 | 809 | + | 1.000 | 0.980 | aTGTAtctc |
| | TEIL.01 | | | | | | | | |
| SEQ_7 | P$MADS/ | Agamous, required for normal flower | 0.80 | 813 | 833 | − | 0.902 | 0.825 | gttTGCCaca |
| | AG.01 | development, similarity to SRF (human) and | | | | | | | tgtgtaagaa |
| | | MCM (yeast) proteins | | | | | | | |
| | | | | | | | | | g |
| SEQ_7 | P$ABRE/ | ABA (abscisic acid) inducible transcriptional | 0.79 | 816 | 832 | + | 0.750 | 0.790 | cttacACATg |
| | ABF1.01 | activator | | | | | | | tggcaaa |
| SEQ_7 | P$OPAQ/ | Recognition site for BZIP transcription | 0.81 | 816 | 832 | − | 1.000 | 0.841 | tttgccACAT |
| | O2_GCN4.01 | factors that belong to the group of | | | | | | | gtgtaag |
| | | Opaque-2 like proteins | | | | | | | |
| SEQ_7 | P$DOFF/ | Prolamin box, conserved in cereal seed storage | 0.75 | 846 | 862 | + | 0.761 | 0.776 | gcacttgtAA |
| | PBOX.01 | protein gene promoters | | | | | | | AAtcaaa |
| SEQ_7 | O$RPOA/ | Avian C-type LTR PolyA signal | 0.71 | 858 | 878 | − | 1.000 | 0.816 | gtaaaTAAAc |
| | APOLYA.01 | | | | | | | | taactttttg |
| | | | | | | | | | a |
| SEQ_7 | P$MYBL/ | Myb-like protein of Petunia hybrida | 0.76 | 861 | 877 | + | 1.000 | 0.919 | aaaagtTAGT |
| | MYBPH3.02 | | | | | | | | ttattta |
| SEQ_7 | P$MADS/ | Binding sites for AP1, AP3-PI and AG dimers | 0.75 | 867 | 887 | − | 1.000 | 0.752 | acaaaCCATg |
| | MADS.01 | | | | | | | | taaataaact |
| | | | | | | | | | a |
| SEQ_7 | P$TBPF/ | Plant TATA box | 0.88 | 869 | 883 | − | 0.782 | 0.886 | accaTGTAaa |
| | TATA.01 | | | | | | | | taaac |
| SEQ_7 | P$OCSE/ | bZIP transcription factor binding to | 0.73 | 908 | 928 | − | 0.974 | 0.753 | taacacaaac |
| | OCSTF.01 | OCS-elements | | | | | | | aacTACGtag |
| | | | | | | | | | g |
| SEQ_7 | P$LFYB/ | Plant specific floral meristem identity gene | 0.93 | 933 | 945 | − | 0.885 | 0.962 | gGCCAttggt |
| | LFY.01 | LEAFY (LFY) | | | | | | | gGCCAttggt |
| SEQ_7 | P$MSAE/ | M-phase-specific activators (NtmybA1, | 0.80 | 934 | 948 | + | 0.750 | 0.800 | aaaccAATGg |
| | MSA.01 | NtmybA2, NtmybB) | | | | | | | ccata |
| SEQ_7 | P$CAAT/ | CCAAT-box in plant promoters | 0.97 | 935 | 943 | + | 1.000 | 0.986 | aaCCAAtgg |
| | CAAT.01 | | | | | | | | |
| SEQ_7 | P$HEAT/ | Heat shock element | 0.81 | 959 | 973 | − | 1.000 | 0.856 | agatatttga |
| | HSE.01 | | | | | | | | AGAAt |
| SEQ_7 | P$MYCL/ | ICE (inducer of CBF expression 1), AtMYC2 | 0.95 | 980 | 998 | + | 1.000 | 0.970 | ttcatACATa |
| | ICE.01 | (rd22BP1) | | | | | | | tgtctaaca |
| SEQ | P$MYBL/M | | 0.76 | 1018 | 1034 | − | 1.000 | 0.896 | cgtggtTAGT |
| _7 | YBPH3.02 | Myb-like protein of Petunia hybrida | | | | | | | taataac |
| SEQ_7 | P$OCSE/ | OCS-like elements | 0.69 | 1018 | 1038 | + | 1.000 | 0.706 | gttattaact |
| | OCSL.01 | | | | | | | | aaccACGTaa |
| | | | | | | | | | a |
| SEQ_7 | P$MIIG/ | Putative cis-acting element in various PAL and | 0.81 | 1021 | 1035 | − | 0.936 | 0.855 | acGTGGttag |
| | PALBOXP.01 | 4CL gene promoters | | | | | | | ttaat |
| SEQ_7 | P$GBOX/ | bZIP protein G-Box binding factor 1 | 0.94 | 1023 | 1043 | − | 1.000 | 0.972 | acatttttAC |
| | GBF1.01 | | | | | | | | GTggttagtta |
| | | | | | | | | | a |
| SEQ_7 | P$GBOX/ | UPRE (unfolded protein response element) | 0.86 | 1024 | 1044 | + | 1.000 | 0.914 | aactaaCCAC |
| | UPRE.01 | like motif | | | | | | | gtaaaaatgt |
| | | | | | | | | | t |
| SEQ_7 | P$OPAQ/ | Recognition site for BZIP transcription | 0.81 | 1025 | 1041 | − | 0.951 | 0.834 | atttttACGTg |
| | O2_GCN4.01 | factors that belong to the group of | | | | | | | gttagt |
| | | Opaque-2 like proteins | | | | | | | |
| SEQ_7 | P$ABRE/ | ABA response elements | 0.82 | 1026 | 1042 | − | 1.000 | 0.879 | catttttACG |
| | ABRE.01 | | | | | | | | Tggttag |
| SEQ_7 | O$RPOA/ | Mammalian C-type LTR Poly A signal | 0.76 | 1062 | 1082 | + | 1.000 | 0.797 | caaaaTAAAc |
| | POLYA.01 | | | | | | | | cataacaatg |
| | | | | | | | | | t |
| SEQ_7 | P$TELO/ | Arabidopsis Telo-box interacting protein | 0.85 | 1066 | 1080 | + | 0.750 | 0.856 | ataaACCAta |
| | ATPURA.01 | related to the conserved animal protein | | | | | | | acaat |
| | | Pur-alpha | | | | | | | |
| SEQ_7 | P$LEGB/ | Legumin box, highly conserved sequence | 0.59 | 1070 | 1096 | + | 0.750 | 0.618 | accataaCAA |
| | LEGB.01 | element about 100 by upstream of the | | | | | | | Tgtgaggata |
| | | TSS in legumin genes | | | | | | | |
| | | | | | | | | | caaatta |
| SEQ_7 | P$DOFF/ | Dof1/MNB1a - single zinc finger | 0.98 | 1088 | 1104 | + | 1.000 | 0.987 | tacaaattAA |
| | DOF1.01 | transcription factor | | | | | | | AGttaca |
| SEQ_7 | P$GTBX/ | Trihelix DNA-binding factor GT-3a | 0.83 | 1093 | 1109 | + | 1.000 | 0.902 | attaaaGTTA |
| | GT3A.01 | | | | | | | | caagttt |
| SEQ_7 | P$GBOX/ | UPRE (unfolded protein response element) | 0.86 | 1134 | 1154 | − | 1.000 | 0.918 | atgcaaCCAC |
| | UPRE.01 | like motif | | | | | | | gtaagagagt |
| | | | | | | | | | g |
| SEQ_7 | P$GBOX/ | bZIP protein G-Box binding factor 1 | 0.94 | 1135 | 1155 | + | 1.000 | 0.973 | actctcttAC |
| | GBF1.01 | | | | | | | | GTggttgcat |
| | | | | | | | | | a |
| SEQ_7 | P$ABRE/ | ABA response elements | 0.82 | 1136 | 1152 | + | 1.000 | 0.836 | ctctcttACG |
| | ABRE.01 | | | | | | | | Tggttgc |
| SEQ_7 | P$OCSE/ | bZIP transcription factor binding to | 0.73 | 1140 | 1160 | − | 0.846 | 0.781 | acacgtatgc |
| | OCSTF.01 | OCS-elements | | | | | | | aacCACGtaag |
| | | | | | | | | | g |
| SEQ_7 | P$OCSE/ | bZIP transcription factor binding to | 0.73 | 1141 | 1161 | + | 0.974 | 0.793 | ttacgtggtt |
| | OCSTF.01 | OCS-elements | | | | | | | gcaTACGtgt |
| | | | | | | | | | g |
| SEQ_7 | P$GBOX/ | bZIP protein G-Box binding factor 1 | 0.94 | 1147 | 1167 | + | 1.000 | 0.968 | ggttgcatAC |
| | GBF1.01 | | | | | | | | GTgtgtatatg |
| | | | | | | | | | g |
| SEQ_7 | P$ABRE/ | ABA response elements | 0.82 | 1148 | 1164 | + | 1.000 | 0.822 | gttgcatACG |
| | ABRE.01 | | | | | | | | Tgtgtat |
| SEQ_7 | P$OCSE/ | OCS-like elements | 0.69 | 1152 | 1172 | − | 1.000 | 0.708 | aaatgcatat |
| | OCSL.01 | | | | | | | | acacACGTat |
| | | | | | | | | | g |
| SEQ_7 | P$MYBS/ | MYB protein from wheat | 0.83 | 1154 | 1170 | − | 1.000 | 0.917 | atgcATATac |
| | TAMYB80.01 | | | | | | | | acacgta |
| SEQ_7 | P$MYBS/ | MYB protein from wheat | 0.83 | 1159 | 1175 | + | 1.000 | 0.893 | gtgtATATgc |
| | TAMYB80.01 | | | | | | | | atttatg |
| SEQ_7 | P$L1BX/ | L1-specific homeodomain protein ATML1 | 0.82 | 1163 | 1179 | − | 1.000 | 0.938 | ctctcaTAAA |
| | ATML1.01 | (A. thaliana meristem layer 1) | | | | | | | tgcatat |
| SEQ_7 | P$DOFF/ | Dof1/MNB1a-single zinc finger transcription | 0.98 | 1174 | 1190 | + | 1.000 | 0.982 | tgagagctAA |
| | DOF1.01 | factor | | | | | | | AGagtat |
| SEQ_7 | P$MYBS/ | Rice MYB proteins with single DNA binding | 0.82 | 1183 | 1199 | + | 1.000 | 0.905 | aagagTATCc |
| | OSMYBS.01 | domains, binding to the amylase element) | | | | | | | attcatt |
| | | (TATCCA) | | | | | | | |
| SEQ_7 | P$MYBS/ | MYB protein from wheat | 0.83 | 1194 | 1210 | − | 1.000 | 0.896 | aagtATATgc |
| | TAMYB80.01 | | | | | | | | aaatgaa |
| SEQ_7 | P$L1BX/ | L1-specific homeodomain protein ATML1 | 0.82 | 1197 | 1213 | − | 0.750 | 0.827 | tgaaagTATA |
| | ATML1.01 | (A. thaliana meristem layer 1) | | | | | | | tgcaaat |
| SEQ_7 | P$MYBS/ | MYB protein from wheat | 0.83 | 1199 | 1215 | + | 1.000 | 0.909 | ttgcATATac |
| | TAMYB80.01 | | | | | | | | tttcata |
| SEQ_7 | P$OCSE/ | OCS-like elements | 0.69 | 1212 | 1232 | − | 1.000 | 0.692 | agagttatat |
| | OCSL.01 | | | | | | | | ataaACGTat |
| | | | | | | | | | g |
| SEQ_7 | P$TBPF/ | Plant TATA box | 0.88 | 1213 | 1227 | − | 1.000 | 0.889 | tataTATAaa |
| | TATA.01 | | | | | | | | cgtat |
| SEQ_7 | P$TBPF/ | Plant TATA box | 0.90 | 1215 | 1229 | − | 1.000 | 0.917 | gttaTATAta |
| | TATA.02 | | | | | | | | aacgt |
| SEQ_7 | P$TBPF/ | Plant TATA box | 0.90 | 1216 | 1230 | + | 1.000 | 0.937 | cgttTATAta |
| | TATA.02 | | | | | | | | taact |
| SEQ_7 | P$TBPF/ | Plant TATA box | 0.90 | 1217 | 1231 | − | 1.000 | 0.931 | gagtTATAta |
| | TATA.02 | | | | | | | | taaac |
| SEQ_7 | O$RPAD/ | Mammalian C-type LTR Poly A downstream | 0.87 | 1242 | 1254 | − | 1.000 | 0.877 | attGTGGttt |
| | PADS.01 | element | | | | | | | cat |
| SEQ_7 | P$DOFF/ | Prolamin box, conserved in cereal seed | | | | | | | tggattagAA |
| | PBOX.01 | storage protein gene promoters | | | | | | | AGtgtat |
| SEQ_7 | P$CAAT/ | CCAAT-box in plant promoters | 0.97 | 1267 | 1275 | + | 1.000 | 0.992 | atCCAAtaa |
| | CAAT.01 | | | | | | | | |
| SEQ_7 | P$AHBP/ | Arabidopsis thaliana homeo box protein 1 | 0.90 | 1269 | 1279 | − | 1.000 | 0.990 | agaATTAttg |
| | ATHB1.01 | | | | | | | | g |
| SEQ_7 | P$AHBP/ | HDZip class I protein ATHB5 | 0.89 | 1269 | 1279 | + | 0.829 | 0.940 | ccaATAAttc |
| | ATHB5.01 | | | | | | | | t |
| SEQ_7 | P$MYBS/ | Rice MYB proteins with single DNA binding | 0.82 | 1277 | 1293 | − | 1.000 | 0.827 | aaaggTATCa |
| | OSMYBS.01 | domains, binding to the amylase element | | | | | | | atagaga |
| | | (TATCCA) | | | | | | | |

TABLE 2
cis-regulatory elements of SEQ ID NO: 8

| | | | | | | | | | |
|---|---|---|---|---|---|---|---|---|---|
| SEQ_8 | P$MADS/ | AGL15, Arabidopsis MADS-domain protein | 0.79 | 16 | 36 | + | 1.000 | 0.822 | cagTACTaca |
| | AGL15.01 | AGAMOUS-like 15 | | | | | | | tttggtatca |
| | | | | | | | | | a |
| SEQ_8 | P$MYCL/ | ICE (inducer of CBF expression 1), AtMYC2 (rd22BP1) | 0.95 | 16 | 34 | − | 1.000 | 0.988 | gtactACATt |
| | ICE.01 | | | | | | | | tggtatcaa |
| SEQ_8 | P$MADS/ | AGL3, MADS Box protein | 0.83 | 17 | 37 | + | 0.973 | 0.929 | tgataCCAAa |
| | AGL3.01 | | | | | | | | tgtagtactg |
| | | | | | | | | | t |
| SEQ_8 | P$SPF1/ | DNA-binding protein of sweet potato that binds to | 0.87 | 30 | 42 | + | 1.000 | 0.883 | agTACTgtgg |
| | SP8BF.01 | the SP8a (ACTGTGTA) and SP8b (TACTATT) sequences | | | | | | | tgt |
| | | of sporamin and beta-amylase genes | | | | | | | |
| SEQ_8 | P$DOFF/ | Prolamin box, conserved in cereal seed storage | 0.75 | 35 | 51 | + | 0.776 | 0.796 | tgtggtgtAA |
| | PBOX.01 | protein gene promoters | | | | | | | ATcctct |
| SEQ_8 | P$L1BX/ | L1-specific homeodomain protein ATML1 (A. thaliana | 0.82 | 52 | 68 | + | 1.000 | 0.834 | gtttacTAAA |
| | ATML1.01 | meristem layer 1) | | | | | | | tgcttcc |
| SEQ_8 | P$GTBX/ | S1F, site 1 binding factor of spinach rps1 promoter | 0.79 | 58 | 74 | − | 1.000 | 0.797 | ctgtATGGaa |
| | S1F.01 | | | | | | | | gcattta |
| SEQ_8 | P$MADS/ | AGL1, Arabidopsis MADS-domain protein | 0.84 | 103 | 123 | − | 1.000 | 0.850 | ttcTGCCcct |
| | AGL1.01 | AGAMOUS-like 1 | | | | | | | gtcggaaatg |
| | | | | | | | | | c |
| SEQ_8 | P$DREB/ | C-repeat/dehydration response element | 0.89 | 104 | 118 | + | 1.000 | 0.923 | catttCCGAc |
| | CRT_DRE.01 | | | | | | | | agggg |
| SEQ_8 | P$MADS/ | AGL1, Arabidopsis MADS-domain protein | 0.84 | 104 | 124 | + | 0.975 | 0.856 | catTTCCgac |
| | AGL1.01 | AGAMOUS-like 1 | | | | | | | aggggcagaa |
| | | | | | | | | | c |
| SEQ_8 | P$LEGB/ | RY and Sph motifs conserved in seed-specific | 0.87 | 154 | 180 | − | 1.000 | 0.873 | gagacattCA |
| | RY.01 | promoters | | | | | | | TGcaccaggc |
| | | | | | | | | | gggctgt |
| SEQ_8 | P$NCS3/ | Nodulin consensus sequence 3 | 0.89 | 203 | 213 | + | 1.000 | 0.960 | gtCACCctcc |
| | NCS3.01 | | | | | | | | c |
| SEQ_8 | P$MADS/ | AGL2, Arabidopsis MADS-domain protein | 0.82 | 239 | 259 | + | 0.763 | 0.821 | caaatCCGTa |
| | AGL | AGAMOUS-like 2 | | | | | | | actcgtaaat |
| | | | | | | | | | a |
| SEQ_8 | P$OCSE/ | bZIP transcription factor binding to OCS-elements | 0.73 | 248 | 268 | − | 0.974 | 0.770 | tggcggtagt |
| | OCSTF.01 | | | | | | | | attTACGagt |
| | | | | | | | | | t |
| SEQ_8 | P$DREB/ | H. vulgare dehydration-response factor 1 | 0.89 | 258 | 272 | + | 1.000 | 0.897 | tactACCGcc |
| | HVDRF1.01 | | | | | | | | accgg |
| SEQ_8 | P$CE1F/ | ABA insensitive protein 4 (ABI4) | 0.87 | 263 | 275 | + | 1.000 | 0.893 | ccgcCACCgg |
| | ABI4.01 | | | | | | | | cca |
| SEQ_8 | 0$RPOA/ | Avian C-type LTR PolyA signal | 0.71 | 264 | 284 | − | 0.750 | 0.754 | agaaaAAAAt |
| | APOLYA.01 | | | | | | | | ggccggtggc |
| | | | | | | | | | g |
| SEQ_8 | P$MADS/ | MADS-box protein SQUAMOSA | 0.90 | 268 | 288 | + | 1.000 | 0.902 | accggccATT |
| | SQUA.01 | | | | | | | | Tttttcttag |
| | | | | | | | | | a |
| SEQ_8 | P$LREM/ | Motif involved in carotenoid and tocopherol | 0.85 | 281 | 291 | − | 1.000 | 0.926 | aaATCTaaga |
| | ATCTA.01 | biosynthesis and in the expression of | | | | | | | a |
| | | photosynthesis-related genes | | | | | | | |
| SEQ_8 | P$CCAF/ | Circadian clock associated 1 | 0.85 | 284 | 298 | − | 1.000 | 0.981 | caaaaaaaAA |
| | CCA1.01 | | | | | | | | TCtaa |
| SEQ_8 | P$NCS1/ | Nadulin consensus sequence 1 | 0.85 | 298 | 308 | − | 1.000 | 0.949 | aAAAAgattt |
| | NCS1.01 | | | | | | | | c |
| SEQ_8 | P$CCAF/ | Circadian clock associated 1 | 0.85 | 312 | 326 | − | 1.000 | 0.860 | gaaaaaaaAA |
| | CCA1.01 | | | | | | | | TCaga |
| SEQ_8 | P$CCAF/ | Circadian clock associated 1 | 0.85 | 325 | 339 | − | 1.000 | 0.855 | gaaaaaaaAA |
| | CCA1.01 | | | | | | | | TCcga |
| SEQ_8 | P$MSAE/ | M-phase-specific activators (NtmybA1, | 0.80 | 337 | 351 | − | 1.000 | 0.854 | acacaAACGg |
| | MSA.01 | NtmybA2, NtmybB) | | | | | | | gagaa |
| SEQ_8 | P$CARM/ | CA-rich element | 0.78 | 343 | 361 | − | 1.000 | 0.791 | tcctcacAAC |
| | CARICH.01 | | | | | | | | Acacaaacg |
| SEQ_8 | P$MYBL/ | CAACTC regulatory elements, GA-inducible | 0.83 | 359 | 375 | + | 1.000 | 0.891 | ggaagagAGT |
| | CARE.01 | | | | | | | | Tgtggga |
| SEQ_8 | P$URNA/ | Upstream sequence elements in the promoters of | 0.75 | 363 | 379 | − | 1.000 | 0.774 | catttcCCAC |
| | USE.01 | U-snRNA genes of higher plants | | | | | | | aactctc |
| SEQ_8 | P$OCSE/ | OCS-like elements | 0.69 | 366 | 386 | + | 0.807 | 0.690 | agttgtggga |
| | OCSL.01 | | | | | | | | aatgACATat |
| | | | | | | | | | a |
| SEQ_8 | P$OPAQ/ | Recognition site for BZIP transcription | 0.81 | 374 | 390 | + | 1.000 | 0.837 | gaaatgACAT |
| | O2_GCN4.01 | factors that belong to the group of | | | | | | | atatata |
| | | Opaque- like proteins | | | | | | | |
| SEQ_8 | P$TBPF/ | Plant TATA box | 0.90 | 379 | 393 | + | 1.000 | 0.938 | gacaTATAta |
| | TATA.02 | | | | | | | | tagag |
| SEQ_8 | P$TBPF/ | Plant TATA box | 0.90 | 380 | 394 | − | 1.000 | 0.960 | tctcTATAta |
| | TATA.02 | | | | | | | | tatgt |
| SEQ_8 | P$PSRE/ | GAAA motif involved in pollen specific | 0.83 | 388 | 404 | + | 1.000 | 0.932 | atagaGAAAt |
| | GAAA.01 | transcriptional activation | | | | | | | tttcgag |
| SEQ_8 | P$MYBL/ | CAACTC regulatory elements, GA-inducible | 0.83 | 396 | 412 | + | 1.000 | 0.588 | attttcgAGT |
| | CARE.01 | | | | | | | | Tgggtag |
| SEQ_8 | P$MIIG/ | Maize C1 myb-domain protein | 0.92 | 404 | 418 | + | 1.000 | 0.957 | gttggGTAGt |
| | MYBC1.01 | | | | | | | | tgaaa |
| SEQ_8 | P$MYBL/ | CAACTC regulatory elements, GA-inducible | 0.83 | 404 | 420 | + | 1.000 | 0.866 | gttgggtAGT |
| | CARE.01 | | | | | | | | Tgaaata |
| SEQ_8 | P$MYBL/ | GA-regulated myb gene from barley | 0.91 | 416 | 432 | − | 1.000 | 0.924 | aaatcgttGT |
| | GAMYB.01 | | | | | | | | TAtattt |
| SEQ_8 | P$MYBS/ | MYB protein from wheat | 0.83 | 432 | 448 | − | 1.000 | 0.845 | taaaATATtc |
| | TAMYB80.01 | | | | | | | | cttataa |
| SEQ_8 | P$OPAQ/ | Recognition site for BZIP transcription | 0.81 | 442 | 458 | + | 1.000 | 0.872 | tattttACAT |
| | O2_GCN4.01 | factors that belong to the group of | | | | | | | ggatttt |
| | | Opaque-2 like proteins | | | | | | | |
| SEQ_8 | P$GTBX/ | S1F, site 1 binding factor of spinach rpsl | 0.79 | 446 | 462 | + | 1.000 | 0.805 | ttacATGGat |
| | S1F.01 | promoter | | | | | | | tttacat |
| SEQ_8 | P$OPAQ/ | Recognition site for BZIP transcription | 0.81 | 453 | 469 | + | 1.000 | 0.846 | gattttACAT |
| | O2_GCN4.01 | factors that belong to the group of | | | | | | | ggtttta |
| | | Opaque-2 like proteins | | | | | | | |
| SEQ_8 | P$GTBX/ | S1F, site 1 binding factor of spinach rps1 | 0.79 | 457 | 473 | + | 1.000 | 0.807 | ttacATGGtt |
| | S1F.01 | promoter | | | | | | | ttaacat |
| SEQ_8 | P$NACF/ | Wheat NACdomain DNA binding factor | 0.68 | 483 | 505 | + | 0.812 | 0.688 | aacgacttta |
| | TANAC69.01 | | | | | | | | cGACGaaatt |
| | | | | | | | | | agg |
| SEQ_8 | P$MADS/ | Agamous, required for normal flower development, | 0.80 | 490 | 510 | − | 1.000 | 9,834 | actTACCtaa |
| | AG.01 | sililarity to SRF (human) and MCM (yeast) | | | | | | | tttcgtcgta |
| | | proteins | | | | | | | a |
| SEQ_8 | P$GTBX/ | GT1-Box binding factors with a trihelix DNA- | 0.85 | 499 | 515 | + | 0.968 | 0.907 | aattagGTAA |
| | GT1.01 | binding domain | | | | | | | gttaaag |
| SEQ_8 | P$DOFF/ | Dof2-single zinc finger transcription factor | 0.98 | 504 | 520 | + | 1.000 | 0.993 | ggtaagttAA |
| | DOF2.01 | | | | | | | | AGcacat |
| SEQ_8 | P$L1BX/ | L1-specific homeodomain protein ATML1 | 0.82 | 513 | 529 | − | 1.000 | 0.838 | gtgtatTAAA |
| | ATML1.01 | (A. thaliana meristem layer 1) | | | | | | | tgtgctt |
| SEQ_8 | P$GBOX/ | bZIP protein G-Box binding factor 1 | 0.94 | 519 | 539 | − | 1.000 | 0.956 | aggtgaaaAC |
| | GBF1.01 | | | | | | | | GTgtattaaa |
| | | | | | | | | | t |
| SEQ_8 | P$OCSE/OCS | OCS-like elements | 0.69 | 525 | 545 | − | 1.000 | 0.777 | atatttaggt |
| | OCSL.01 | | | | | | | | gaaaACGTgt |
| | | | | | | | | | a |
| SEQ_8 | P$GTBX/ | Trihelix DNA-binding factor GT-3a | 0.83 | 538 | 554 | − | 1.000 | 0.839 | gttaccGTTA |
| | GT3A.01 | | | | | | | | tatttag |
| SEQ_8 | P$MYBL/ | Myb-like protein of Petunia hybrida | 0.80 | 540 | 556 | − | 1.000 | 0.837 | atgttaccGT |
| | MYBPH3.01 | | | | | | | | TAtattt |
| SEQ_8 | P$MSAE/ | M-phase-specific activators (NtmybA1, | 0.80 | 541 | 555 | + | 1.000 | 0.942 | aatatAACGg |
| | MSA.01 | NtmybA2, NtmybB) | | | | | | | taaca |
| SEQ_8 | P$GTBX/ | Trihelix DNA-binding factor GT-3a | 0.83 | 544 | 560 | − | 1.000 | 0.876 | aagcatGTTA |
| | GT3A.01 | | | | | | | | ccgttat |
| SEQ_8 | P$NACF/ | Wheat NACdomain DNA binding factor | 0.68 | 573 | 595 | + | 1.000 | 0.692 | gatgtaatga |
| | TANAC69.01 | | | | | | | | tTACGacgta |
| | | | | | | | | | ttt |
| SEQ_8 | P$AHBP/ | HD-ZIP class III protein ATHB9 | 0.77 | 576 | 586 | + | 1.000 | 1.000 | gtaATGAtta |
| | ATHB9.01 | | | | | | | | c |
| SEQ_8 | P$AHBP/ | Sunflower homeodomain leucine-zipper | 0.87 | 576 | 586 | − | 1.000 | 0.981 | gtaatcATTA |
| | HAHB4.01 | protein Hahb-4 | | | | | | | c |
| SEQ_8 | P$GBOX/ | Anaerobic basic leucine zipper | 0.91 | 595 | 615 | − | 1.000 | 0.910 | acgaatgtAC |
| | ABZ1.01 | | | | | | | | GTggaatgag |
| | | | | | | | | | a |
| SEQ_8 | P$OPAQ/ | Opaque-2 regulatory protein | 0.87 | 598 | 614 | + | 1.000 | 0.901 | cattCCACgt |
| | O2.02 | | | | | | | | acattcg |
| SEQ_8 | P$EINL/ | TEIL (tobacco EIN3-like) | 0.92 | 603 | 611 | − | 1.000 | 0.941 | aTGTAcgtg |
| | TEIL.01 | | | | | | | | |
| SEQ_8 | P$NACF/ | Wheat NACdomain DNA binding factor | 0.68 | 627 | 649 | + | 0.812 | 0.681 | aaggagttta |
| | TANAC69.01 | | | | | | | | cGACGaaacc |
| | | | | | | | | | agt |
| SEQ_8 | P$DOFF/ | Prolamin box, conserved in cereal seed storage | 0.75 | 654 | 670 | − | 0.776 | 0.768 | cgccatgtAA |
| | PBOX.01 | protein gene promoters | | | | | | | ATttacg |
| SEQ_8 | P$GBOX/ | Oryza sativa bZIP protein 8 | 0.84 | 655 | 675 | + | 0.750 | 0.843 | gtaaatttAC |
| | OSBZ8.01 | | | | | | | | ATggcgttta |
| | | | | | | | | | c |
| SEQ_8 | P$LREM/ | Motif involved in carotenoid and tocopherol | 0.85 | 681 | 691 | − | 1.000 | 0.900 | gaATCTaact |
| | ATCTA.01 | biosynthesis and in the expression of | | | | | | | t |
| | | photosynthesis-related genes | | | | | | | |
| SEQ_8 | P$DOFF/ | Dof3-single zinc finger transcription factor | 0.99 | 700 | 716 | + | 1.000 | 0.995 | ttcgttgtAA |
| | DOF3.01 | | | | | | | | AGcccat |
| SEQ_8 | P$ERSE/ | ERSE I (ER stress-response element I)-like | 0.79 | 712 | 730 | + | 0.750 | 0.791 | cccatgtaaa |
| | ERSE_I.01 | motif | | | | | | | tttacGACG |
| SEQ_8 | P$MYBL/ | Myb-like protein of Petunia hybrida | 0.76 | 746 | 762 | + | 0.778 | 0.761 | aaatgtTCGT |
| | MYBPH3.02 | | | | | | | | tgttatg |
| SEQ_8 | P$MYBL/ | GA-regulated myb gene from barley | 0.91 | 749 | 765 | + | 1.000 | 0.946 | tgttcgttGT |
| | GAMYB.01 | | | | | | | | TAtgggc |
| SEQ_8 | P$GTBX/ | S1F, site 1 binding factor of spinach rpsl | 0.79 | 756 | 772 | + | 1.000 | 0.795 | tgttATGGgc |
| | S1F.01 | promoter | | | | | | | acgtttt |
| SEQ_8 | P$GBOX/ | Oryza sativa bZIP protein 8 | 0.84 | 757 | 777 | − | 1.000 | 0.865 | acaagaaaAC |
| | OSBZ8.01 | | | | | | | | GTgcccataa |
| | | | | | | | | | c |
| SEQ_8 | P$MYCL/ | Myc recognition sequences | 0.93 | 758 | 776 | − | 1.000 | 0.940 | caagaaaACG |
| | MYCRS.01 | | | | | | | | Tgcccataa |
| SEQ_8 | P$ABRE/ | ABA (abscisic acid) inducible transcriptional | 0.82 | 760 | 776 | − | 1.000 | 0.892 | caagaaaaCG |
| | ABF1.03 | activator | | | | | | | TGcccat |
| SEQ_8 | P$OCSE/ | OCS-like elements | 0.69 | 763 | 783 | − | 1.000 | 0.691 | atcactacaa |
| | OCSL.01 | | | | | | | | gaaaACGTgc |
| | | | | | | | | | c |
| SEQ_8 | P$AHBP/ | Arabidopsis thaliana homeo box protein 1 | 0.90 | 795 | 805 | + | 1.000 | 0.989 | aaaATTAtta |
| | ATHB1.01 | | | | | | | | a |
| SEQ_8 | P$AHBP/ | HDZip class I protein ATHB5 | 0.89 | 795 | 805 | − | 0.829 | 0.902 | ttaATAAttt |
| | ATHB5.01 | | | | | | | | t |
| SEQ_8 | P$GTBX/ | SBF-1 | 0.87 | 795 | 811 | + | 1.000 | 0.886 | aaattaTTAA |
| | SBF1.01 | | | | | | | | aaaaaa |
| SEQ_8 | P$NCS1/ | Nadulin consensus sequence 1 | 0.85 | 807 | 817 | + | 1.000 | 0.990 | aAAAAgatta |
| | NCS1.01 | | | | | | | | a |
| SEQ_8 | P$AHBP/ | Homeodomain protein WUSCHEL | 0.94 | 811 | 821 | − | 1.000 | 0.963 | gttctTAATc |
| | WUS.01 | | | | | | | | t |
| SEQ_8 | P$MADS/ | AGL3, MADS Box protein | 0.83 | 824 | 844 | − | 0.973 | 0.854 | ttatcCCAAa |
| | AGL3.01 | | | | | | | | tactgattta |
| | | | | | | | | | g |
| SEQ_8 | P$MYBS/ | MybSt1 (Myb Solanum tuberosum 1) with a single | 0.90 | 832 | 848 | − | 1.000 | 0.957 | atcattATCC |
| | MYBST1.01 | myb repeat | | | | | | | caaatac |
| SEQ_8 | P$1BOX/ | Class I GATA factors | 0.93 | 835 | 851 | + | 1.000 | 0.932 | tttggGATAa |
| | GATA.01 | | | | | | | | tgatgct |
| SEQ_8 | P$AHBP/ | Sunflower homeodomain leucine-zipper | 0.87 | 841 | 851 | − | 1.000 | 0.937 | agcatcATTA |
| | HAHB4.01 | protein Hahb-4 | | | | | | | t |
| SEQ_8 | P$MADS/ | Agamous, required for normal flower | 0.80 | 845 | 865 | − | 1.000 | 0.808 | ctaTACCtaa |
| | AG.01 | development, similarity to SRF (human) | | | | | | | taagagcatc |
| | | and MCM (yeast) proteins | | | | | | | |
| | | | | | | | | | a |
| SEQ_8 | P$MIIG/ | Maize C1 myb-domain protein | 0.92 | 871 | 885 | + | 1.000 | 0.980 | cgttgGTAGt |
| | MYBC1.01 | | | | | | | | tgcat |
| SEQ_8 | P$MYBL/ | CAACTC regulatory elements, GA-inducible | 0.83 | 871 | 887 | + | 1.000 | 0.844 | cgttggtAGT |
| | CARE.01 | | | | | | | | Tgcattg |
| SEQ_8 | P$NCS1/ | Nadulin consensus sequence 1 | 0.85 | 889 | 899 | − | 0.878 | 0.909 | cAAATgatga |
| | NCS1.01 | | | | | | | | t |
| SEQ_8 | P$GBOX/ | HBP-1a, suggested to be involved in the cell | 0.88 | 896 | 916 | − | 1.000 | 0.919 | tgcatgcCAC |
| | HBP1A.01 | cycle-dependent expression | | | | | | | Gttgaatcaa |
| | | | | | | | | | a |
| SEQ_8 | P$GBOX/ | Oryza sativa bZIP protein 8 | 0.84 | 897 | 917 | + | 1.000 | 0.941 | ttgattcaAC |
| | OSBZ8.01 | | | | | | | | GTggcatgca |
| | | | | | | | | | g |
| SEQ_8 | P$ABRE/ | ABA response elements | 0.82 | 898 | 914 | + | 1.000 | 0.939 | tgattcaACG |
| | ABRE.01 | | | | | | | | Tggcatg |
| SEQ_8 | P$LEGB/ | RY and Sph motifs conserved in seed-specific | 0.87 | 903 | 929 | + | 1.000 | 0.884 | caacgtggCA |
| | RY.01 | promoters | | | | | | | TGcagttcat |
| | | | | | | | | | tggctaa |
| SEQ_8 | P$EPFF/ | Member of the EPF family of zinc finger | 0.75 | 904 | 926 | + | 1.000 | 0.751 | aacgtggcat |
| | ZPT22.01 | transcription factors | | | | | | | gCAGTtcatt |
| | | | | | | | | | ggc |
| SEQ_8 | P$CAAT/ | CCAAT-box in plant promoters | 0.97 | 919 | 927 | − | 1.000 | 0.978 | agCCAAtga |
| | CAAT.01 | | | | | | | | |
| SEQ_8 | P$GTBX/ | Trihelix DNA-binding factor GT-3a | 0.83 | 926 | 942 | + | 1.000 | 0.884 | ctaattGTTA |
| | GT3A.01 | | | | | | | | cacatct |
| SEQ_8 | P$AHBP/ | Sunflower homeodomain leucine-zipper protein | 0.87 | 953 | 963 | − | 1.000 | 0.913 | cgtataATTA |
| | HAHB4.01 | Hahb-4 | | | | | | | g |
| SEQ_8 | P$GBOX/ | bZIP protein G-Box binding factor 1 | 0.94 | 953 | 973 | + | 1.000 | 0.956 | ctaattatAC |
| | GBF1.01 | | | | | | | | GTggtggtga |
| | | | | | | | | | g |
| SEQ_8 | P$ABRE/ | ABA response elements | 0.82 | 954 | 970 | + | 1.000 | 0.874 | taattatACG |
| | ABRE.01 | | | | | | | | Tggtggt |
| SEQ_8 | P$SALT/ | Zinc-finger protein in alfalfa roots, regulates | 0.95 | 958 | 972 | + | 1.000 | 0.955 | tatacgtGGT |
| | ALFIN1.02 | salt tolerance | | | | | | | Ggtga |
| SEQ_8 | P$NACF/ | Wheat NACdomain DNA binding factor | 0.68 | 963 | 985 | + | 1.000 | 0.747 | gtggtggtga |
| | TANAC69.01 | | | | | | | | gTACGtagtt |
| | | | | | | | | | caa |
| SEQ_8 | P$MYBS/ | Rice MYB proteins with single DNA binding domains, | 0.82 | 1009 | 1025 | − | 1.000 | 0.842 | aaaagTATCc |
| | OSMYBS.01 | binding to the amylase element (TATCCA) | | | | | | | accaaaa |
| SEQ_8 | O$RVUP/ | Upstream element of C-type Long Terminal Repeats | 0.76 | 1025 | 1045 | + | 0.761 | 0.805 | tgcataatca |
| | LTRUP.01 | | | | | | | | gTTTTgattg |
| | | | | | | | | | t |
| SEQ_8 | P$LREM/ | Motif involved in carotenoid and tocopherol | 0.85 | 1049 | 1059 | + | 1.000 | 0.990 | atATCTaaat |
| | ATCTA.01 | biosynthesis and in the expression of | | | | | | | c |
| | | photosynthesis-related genes | | | | | | | |
| SEQ_8 | P$LREM/ | Motif involved in carotenoid and tocopherol | 0.85 | 1055 | 1065 | + | 1.000 | 0.874 | aaATCTacgt |
| | ATCTA.01 | biosynthesis and and in the expression of | | | | | | | g |
| | | photosynthesis-related genes | | | | | | | |
| SEQ_8 | P$GBOX/TGA | Arabidopsis leucine zipper protein TGA1 | 0.90 | 1058 | 1078 | + | 0.818 | 0.901 | tctacgTGAT |
| | TGA1.01 | | | | | | | | gtaacgaccg |
| | | | | | | | | | a |
| SEQ_8 | P$GTBX/ | Trihelix DNA-binding factor GT-3a | 0.83 | 1071 | 1087 | + | 0.750 | 0.852 | acgaccGATA |
| | GT3A.01 | | | | | | | | caataaa |
| SEQ_8 | 0$RPOA/ | Lentiviral Poly A signal | 0.94 | 1079 | 1099 | + | 1.000 | 0.941 | tacAATAaac |
| | LPOLYA.01 | | | | | | | | aattctgatt |
| | | | | | | | | | g |
| SEQ_8 | P$STKM/ | Storekeeper (STK), plant specific DNA binding | 0.85 | 1081 | 1095 | + | 1.000 | 0.902 | caaTAAAcaa |
| | STK.01 | protein important for tuber-specific and | | | | | | | ttctg |
| | | sucrose-inducible gene expression | | | | | | | |
| SEQ_8 | 0$RPOA/ | Avian C-type LTR PolyA signal | 0.71 | 1097 | 1117 | + | 0.750 | 0.742 | ttgaaTATAt |
| | APOLYA.01 | | | | | | | | atccatatga |
| | | | | | | | | | t |
| SEQ_8 | P$GTBX/ | S1F, site 1 binding factor of spinach rps1 | 0.79 | 1100 | 1116 | − | 1.000 | 0.811 | tcatATGGat |
| | S1F.01 | promoter | | | | | | | atatatt |
| SEQ_8 | P$MYBS/ | Rice MYB proteins with single DNA binding domains, | 0.82 | 1101 | 1117 | + | 1.000 | 0.882 | atataTATCc |
| | OSMYBS.01 | binding to the amylase element (TATCCA) | | | | | | | atatgat |
| SEQ_8 | P$MADS/ | AGL15, Arabidopsis MADS-domain protein | 0.79 | 1104 | 1124 | − | 1.000 | 0.793 | tcaTACTatc |
| | AGL15.01 | AGAMOUS-like 15 | | | | | | | atatggatat |
| | | | | | | | | | a |
| SEQ_8 | P$ERSE/ | ERSE I (ER stress-response element I)-like motif | 0.79 | 1138 | 1156 | + | 1.000 | 0.842 | caactatggt |
| | ERSE_1.01 | | | | | | | | gttgcCACG |
| SEQ_8 | P$NACF/ | Wheat NACdomain DNA binding factor | 0.68 | 1142 | 1164 | + | 0.895 | 0.687 | tatggtgttg |
| | TANAC69.01 | | | | | | | | cCACGtaacg |
| | | | | | | | | | acc |
| SEQ_8 | P$GBOX/ | bZIP protein G-Box binding factor 1 | 0.94 | 1145 | 1165 | − | 1.000 | 0.973 | tggtcgttAC |
| | GBF1.01 | | | | | | | | GTggcaacac |
| | | | | | | | | | c |
| SEQ_8 | P$GBOX/ | HBP-la, suggested to be involved in the cell | 0.88 | 1146 | 1166 | + | 1.000 | 0.952 | gtgttgcCAC |
| | HBP1A.01 | cycle-dependent expression | | | | | | | Gtaacgacca |
| | | | | | | | | | t |
| SEQ_8 | P$OPAQ/ | Rice transcription activator-1 (RITA), basic | 0.95 | 1147 | 1163 | − | 1.000 | 0.981 | gtcgttACGT |
| | RITA1.01 | leucin zipper protein, highly expressed | | | | | | | ggcaaca |
| | | during seed development | | | | | | | |
| SEQ_8 | P$ABRE/ | ABA (abscisic acid) inducible transcriptional | 0.82 | 1148 | 1164 | − | 1.000 | 0.897 | ggtcgttaCG |
| | ABF1.03 | activator | | | | | | | TGgcaac |
| SEQ_8 | P$OPAQ/ | Rice transcription activator-1 (RITA), basic | 0.95 | 1148 | 1164 | + | 1.000 | 0.985 | gttgccACGT |
| | RITA1.01 | leucin zipper protein, highly expressed | | | | | | | aacgacc |
| | | during seed development | | | | | | | |
| SEQ_8 | P$MYBL/ | Anther-specific myb gene from tobacco | 0.96 | 1152 | 1168 | − | 1.000 | 0.974 | agatggtcGT |
| | NTMYBAS1.01 | | | | | | | | TAcgtgg |
| SEQ_8 | 0$RPOA/ | Lentiviral Poly A signal | 0.94 | 1156 | 1176 | − | 1.000 | 0.989 | gttAATAaag |
| | LPOLYA.01 | | | | | | | | atggtcgtta |
| | | | | | | | | | c |
| SEQ_8 | P$MADS/ | | 0.75 | 1158 | 1178 | + | 1.000 | 0.768 | aacgaCCATc |
| | MADS.01 | Binding sites for AP1, AP3-PI and AG dimers | | | | | | | tttattaacc |
| | | | | | | | | | a |
| SEQ_8 | P$DOFF/ | Dof1/MNB1a-single zinc finger transcription factor | 0.98 | 1162 | 1178 | − | 1.000 | 0.984 | tggttaatAA |
| | DOF1.01 | | | | | | | | AGatggt |
| SEQ_8 | P$GTBX/ | SBF-1 | 0.87 | 1166 | 1182 | − | 1.000 | 0.894 | gtcatggTTA |
| | SBF1.01 | | | | | | | | Ataaaga |
| SEQ_8 | P$GBOX/ | Wheat bZIP transcription factor HBP1B (histone | 0.83 | 1172 | 1192 | − | 1.000 | 0.943 | gttgcggcAC |
| | HBP1B.01 | gene binding protein 1b) | | | | | | | GTcatggtta |
| | | | | | | | | | a |
| SEQ_8 | P$GBOX/ | Arabidopsis leucine zipper protein TGA1 | 0.90 | 1173 | 1193 | + | 1.000 | 0.971 | taaccaTGAC |
| | TGA1.01 | | | | | | | | gtgccgcaac |
| | | | | | | | | | c |
| SEQ_8 | P$ABRE/ | ABA (abscisic acid) inducible transcriptional | 0.82 | 1174 | 1190 | + | 1.000 | 0.863 | aaccatgaCG |
| | ABF1.03 | activator | | | | | | | TGccgca |
| SEQ_8 | P$ERSE/ | ERSE I (ER stress-response element I)-like motif | 0.79 | 1183 | 1200 | − | 1.000 | 0.804 | cctctgtggt |
| | ERSE_1.01 | | | | | | | | tgcggCACG |
| SEQ_8 | P$IDDF/ | Maize INDETERMINATE1 zinc finger protein | 0.92 | 1196 | 1208 | − | 1.000 | 0.927 | gttgTTGTcc |
| | ID1.01 | | | | | | | | tct |
| SEQ_8 | P$MYBL/ | GA-regulated myb gene from barley | 0.91 | 1197 | 1213 | − | 0.884 | 0.911 | tatgtgttGT |
| | GAMYB.01 | | | | | | | | GTcctc |
| SEQ_8 | 0$LTUP/ | Lentiviral TATA upstream element | 0.71 | 1213 | 1235 | + | 1.000 | 0.725 | acagcatcga |
| | TAACC.01 | | | | | | | | gaAACCgcat |
| | | | | | | | | | act |
| SEQ_8 | P$MADS/ | AGL15, Arabidopsis MADS-domain protein | 0.79 | 1229 | 1249 | + | 1.000 | 0.808 | gcaTACTaac |
| | AGL15.01 | AGAMOUS-like 15 | | | | | | | actcgcaaag |
| | | | | | | | | | t |
| SEQ_8 | P$CARM/ | CA-rich element | 0.78 | 1245 | 1263 | + | 1.000 | 0.812 | aaagtgcAAC |
| | CARICH.01 | | | | | | | | Acccaaaac |
| SEQ_8 | O$MINI/MUS | Muscle Initiator Sequence | 0.86 | 1290 | 1308 | + | 1.000 | 0.860 | gcaacaCCAC |
| | MUSCLE_IN1.02 | | | | | | | | gcagctata |
| SEQ_8 | P$OCSE/ | OCS-like elements | 0.69 | 1296 | 1316 | + | 1.000 | 0.690 | ccacgcagct |
| | OCSL.01 | | | | | | | | atacACGTat |
| | | | | | | | | | c |
| SEQ_8 | P$GBOX/ | bZIP protein G-Box binding factor 1 | 0.94 | 1301 | 1321 | − | 1.000 | 0.967 | tagaagatAC |
| | GBF1.01 | | | | | | | | GTgtatagct |
| | | | | | | | | | g |
| SEQ_8 | P$OCSE/ | OCS-like elements | 0.69 | 1307 | 1327 | − | 1.000 | 0.737 | ttagcataga |
| | OCSL.01 | | | | | | | | agatACGTgt |
| | | | | | | | | | a |
| SEQ_8 | P$GARP/ | Type-B response regulator (ARR10), member of the | 0.97 | 1309 | 1317 | − | 1.000 | 0.985 | AGATacgtg |
| | ARR10.01 | GARP-family of plant myb-related DNA binding | | | | | | | |
| | | motifs | | | | | | | |
| SEQ_8 | P$GBOX/ | bZIP protein G-Box binding factor 1 | 0.94 | 1320 | 1340 | − | 1.000 | 0.976 | gacatgacAC |
| | GBF1.01 | | | | | | | | GTgttagcat |
| | | | | | | | | | a |
| SEQ_8 | P$GBOX/ | bZIP protein G-Box binding factor 1 | 0.94 | 1321 | 1341 | + | 1.000 | 0.977 | atgctaacAC |
| | GBF1.01 | | | | | | | | GTgtcatgtc |
| | | | | | | | | | t |
| SEQ_8 | P$MYCL/ | ICE (inducer of CBF expression 1), AtMYC2 | 0.95 | 1321 | 1339 | − | 0.954 | 0.966 | acatgACACg |
| | ICE.01 | (rd22BP1) | | | | | | | tgttagcat |
| SEQ_8 | P$ABRE/ | ABA response elements | 9.82 | 1322 | 1338 | + | 1.000 | 0.951 | tgctaacACG |
| | ABRE.01 | | | | | | | | Tgtcatg |
| SEQ_8 | P$CE3S/ | Coupling element 3 (CE3), non-ACGT ABRE | 0.77 | 1322 | 1340 | + | 0.750 | 0.806 | tgctaaCACG |
| | CE3.01 | | | | | | | | tgtcatgtc |
| SEQ_8 | P$MYCL/ | Rice bHLH protein | 0.85 | 1322 | 1340 | + | 1.000 | 0.901 | tgctaaCACG |
| | OSBHLH66.01 | | | | | | | | tgtcatgtc |
| SEQ_8 | P$ABRE/ | ABA response elements | 0.82 | 1323 | 1339 | − | 1.000 | 0.897 | acatgacACG |
| | ABRE.01 | | | | | | | | Tgttagc |
| SEQ_8 | P$DPBF/ | bZIP factors DPBF-1 and 2 (Dc3 promoter binding | 0.89 | 1326 | 1336 | + | 1.000 | 0.920 | aACACgtgtc |
| | DPBF.01 | factor-1 and 2) | | | | | | | a |
| SEQ_8 | P$DREB/ | C-repeat/dehydration response element | 0.89 | 1341 | 1355 | + | 1.000 | 0.904 | ttgaaCCGAc |
| | CRT_DRE.01 | | | | | | | | caaga |
| SEQ_8 | P$MYCL/ | ICE (inducer of CBF expression 1), AtMYC2 | 0.95 | 1356 | 1374 | − | 1.000 | 0.965 | tcgatACATt |
| | ICE.01 | (rd22BP1) | | | | | | | tgtagtgtg |
| SEQ_8 | O$LDPS/ | Lentiviral Poly A downstream element | 0.89 | 1384 | 1398 | − | 1.000 | 0.900 | gtGTGTatgg |
| | LDSPOLYA.01 | | | | | | | | tcttc |
| SEQ_8 | P$MADS/ | AGL15, Arabidopsis MADS-domain protein | 0.79 | 1398 | 1418 | − | 0.850 | 0.823 | tgtTGCTgtg |
| | AGL15.01 | AGAMOUS-like 15 | | | | | | | taaagaaagt |
| | | | | | | | | | g |
| SEQ_8 | P$DOFF/ | Prolamin box, conserved in cereal seed storage | 0.75 | 1399 | 1415 | − | 1.000 | 0.882 | tgctgtgtAA |
| | PBOX.01 | protein gene promoters | | | | | | | AGaaagt |
| SEQ_8 | P$MADS/ | AGL15, Arabidopsis MADS-domain protein | 0.79 | 1399 | 1419 | + | 0.925 | 0.882 | actTTCTtta |
| | AGL15.01 | AGAMOUS-like 15 | | | | | | | cacagcaaca |
| | | | | | | | | | t |
| SEQ_8 | P$RAV5/ | 5′-part of bipartite RAV1 binding site, | 0.96 | 1412 | 1422 | + | 1.000 | 0.967 | agcAACAtac |
| | RAV1-5.01 | interacting with AP2 domain | | | | | | | a |
| SEQ_8 | P$DOFF/ | Dof2-single zinc finger transcription factor | 0.98 | 1431 | 1447 | − | 1.000 | 0.983 | aattatatAA |
| | DOF2.01 | | | | | | | | AGctttt |
| SEQ_8 | P$TBPF/ | Plant TATA box | 0.88 | 1432 | 1446 | − | 1.000 | 0.892 | attaTATAaa |
| | TATA.01 | | | | | | | | gcttt |
| SEQ_8 | P$TBPF/ | Plant TATA box | 0.90 | 1434 | 1448 | − | 1.000 | 0.921 | taatTATAta |
| | TATA.02 | | | | | | | | aagct |
| SEQ_8 | P$AHBP/ | Sunflower homeodomain leucine-zipper protein | 0.87 | 1439 | 1449 | + | 1.000 | 0.902 | tatataATTA |
| | HAHB4.01 | Hahb-4 | | | | | | | t |
| SEQ_8 | P$1BOX/ | Class I GATA factors | 0.93 | 1439 | 1455 | − | 1.000 | 0.946 | gaaatGATAa |
| | GATA.01 | | | | | | | | ttatata |
| SEQ_8 | P$AHBP/ | Sunflower homeodomain leucine-zipper protein | 0.87 | 1442 | 1452 | − | 1.000 | 0.910 | atgataATTA |
| | HAHB4.01 | Hahb-4 | | | | | | | t |
| SEQ_8 | P$AHBP/ | HD-ZIP class III protein ATHB9 | 0.77 | 1445 | 1455 | − | 1.000 | 0.775 | gaaATGAtaa |
| | ATHB9.01 | | | | | | | | t |
| SEQ_8 | P$NCS1/ | Nodulin consensus sequence 1 | 0.85 | 1445 | 1455 | − | 0.878 | 0.915 | gAAATgataa |
| | NCS1.01 | | | | | | | | t |

TABLE 3
cis-regulatory elements of SEQ ID NO: 9

| | | | | | | | | | |
|---|---|---|---|---|---|---|---|---|---|
| SEQ_9 | P$CGCG/ATSR1.01 | Arabidopsis thaliana signal-responsive | 0.84 | 2 | 18 | − | 1.000 | 0.859 | ccaCGCGtgcc |
| | | gene1, Ca2+/ calmodulin binding protein | | | | | | | ctatagcgac |
| | | homolog to NtER1 (tobacco early ethylene- | | | | | | | |
| | | responsive gene) | | | | | | | |
| SEQ_9 | P$CE3S/CE3.01 | Coupling element 3 (CE3), non-ACGT ABRE | 0.77 | 3 | 21 | − | 1.000 | 0.905 | caCGCGtgccc |
| | | | | | | | | | tata |
| SEQ_9 | P$CGCG/ATSR1.01 | Arabidopsis thaliana signal-responsive | 0.84 | 9 | 25 | + | 1.000 | 0.856 | gcaCGCGtggt |
| | | gene1, Ca2+/ calmodulin binding protein | | | | | | | cgacgg |
| | | homolog to NtER1 (tobacco early ethylene- | | | | | | | |
| | | responsive gene) | | | | | | | |
| SEQ_9 | P$MIIG/P_ACT.01 | Maize activator P of flavonoid | 0.93 | 30 | 44 | + | 0.966 | 0.983 | ggctGGTTggt |
| | | biosynthetic genes | | | | | | | aaaa |
| SEQ_9 | P$GBOX/ROM.01 | Regulator of MAT (ROM1, ROM2) | 0.85 | 39 | 59 | + | 1.000 | 0.891 | gtaaaaCCACc |
| | | | | | | | | | tcagcctccg |
| SEQ_9 | P$GBOX/ | bZIP transcription factor from | 0.84 | 44 | 64 | − | 0.750 | 0.855 | tgaatcggagg |
| | BZIP910.02 | Antirrhinum majus | | | | | | | cTGAGgtggt |
| SEQ_9 | O$RVUP/LTRUP.01 | Upstream element of C-type Long Terminal | 0.76 | 55 | 75 | + | 1.000 | 0.804 | ctccgattcag |
| | | Repeats | | | | | | | TTTCtggatc |
| SEQ_9 | P$GBOX/ | bZIP transcription factor from | 0.76 | 107 | 127 | − | 0.750 | 0.765 | cggcggAGACt |
| | BZIP911.02 | Antirrhinum majus | | | | | | | tgtccttctt |
| SEQ_9 | P$DREB/HVDRF1.01 | H. vulgare dehydration-response factor 1 | 0.89 | 117 | 131 | + | 0.782 | 0.900 | agtctccgccg |
| | | | | | | | | | ccgg |
| SEQ_9 | P$MADS/AGL1.01 | Arabidopsis MADS-domain protein | 0.84 | 130 | 150 | + | 0.761 | 0.860 | ggcAACCAaat |
| | | AGAMOUS-like 1 | | | | | | | cgggaacgaa |
| SEQ_9 | P$E2FF/E2F.01 | E2F class I sites | 0.82 | 136 | 150 | − | 1.000 | 0.825 | ttcgTTCCcga |
| | | | | | | | | | tttg |
| SEQ_9 | P$TCPF/ | TCP class I transcription factor | 0.94 | 150 | 162 | + | 1.000 | 0.944 | agcgGCCCagc |
| | ATTCP20.01 | (Arabidopsis) | | | | | | | ga |
| SEQ_9 | P$CGCG/OSCBT.01 | Oryza sativa CaM-binding transcription | 0.78 | 201 | 217 | − | 0.817 | 0.783 | cttCGAGtctt |
| | | factor | | | | | | | cgccga |
| SEQ_9 | P$IDRE/IDE1.01 | Iron-deficiency-responsive element 1 | 0.77 | 213 | 227 | − | 0.777 | 0.805 | caGCAGgcttc |
| | | | | | | | | | ttcg |
| SEQ_9 | P$DOFF/DOF3.01 | Dof3 - single zinc finger transcription | 0.99 | 223 | 239 | − | 1.000 | 0.979 | gtctctgaAAA |
| | | factor | | | | | | | Gcagca |
| SEQ_9 | P$WBXF/ZAP1.01 | Arabidopsis thaliana Zinc-dependent | 0.84 | 237 | 253 | + | 0.750 | 0.840 | gactgTGGAcc |
| | | Activator Protein-1 (ZAP1) | | | | | | | gaggac |
| SEQ_9 | O$RVUP/LTRYOP.01 | Upstream element of C-type Long Terminal | 0.76 | 275 | 295 | + | 1.000 | 0.769 | ggcatgatcga |
| | | Repeats | | | | | | | TTTCaagaag |
| SEQ_9 | P$MYBS/HVMCB1.01 | Hordeum vulgare Myb-related CAB-promoter- | 0.93 | 288 | 304 | − | 1.000 | 0.957 | ccccgtATCCt |
| | | binding protein 1 | | | | | | | tcttga |
| SEQ_9 | P$OSCE/OCSTF/01 | bZIP transcription factor binding to OCS- | 0.73 | 313 | 333 | + | 0.846 | 0.739 | ttacgacgaca |
| | | elements | | | | | | | ccAACGctta |
| SEQ_9 | P$MSAE/MSA.01 | M-phase-specific activators (NtmybA1, | 0.80 | 321 | 335 | + | 1.000 | 0.802 | acaccAACGct |
| | | NtmybA2, NtmybB) | | | | | | | tact |
| SEQ_9 | O$LTUP/TAACC.01 | Lentiviral TATA upstream element | 0.71 | 360 | 382 | − | 1.000 | 0.718 | ctggttcttgc |
| | | | | | | | | | tAACCtcaaag |
| | | | | | | | | | c |
| SEQ_9 | P$MYBL/GAMYB.01 | GA-regulated myb gene from barley | 0.91 | 360 | 376 | + | 1.000 | 0.914 | gctttgagGTT |
| | | | | | | | | | Agcaag |
| SEQ_9 | P$MYBS/MYBST1.01 | MybSt1 (Myb Solanum tuberosum 1) with a | 0.90 | 381 | 397 | − | 1.000 | 0.958 | aagcttATCCa |
| | | single myb repeat | | | | | | | tgaact |
| SEQ_9 | P$GTBX/S1F.01 | S1F, site 1 binding factor of spinach | 0.79 | 382 | 398 | + | 1.000 | 0.811 | gttcATGGata |
| | | rps1 promoter | | | | | | | agctta |
| SEQ_9 | P$IBOX/GATA.01 | Class I GATA factors | 0.93 | 384 | 400 | + | 1.000 | 0.945 | tcatgGATAag |
| | | | | | | | | | cttagg |
| SEQ_9 | P$CARM/CARICH.01 | CA-rich element | 0.78 | 393 | 411 | − | 0.750 | 0.866 | ttcttcaAACT |
| | | | | | | | | | cctaagct |
| SEQ_9 | P$MIIG/ | Cis-acting element conserved in various | 0.80 | 439 | 453 | − | 1.000 | 0.818 | tgaggcttGGT |
| | PALBOXL.01 | PAL and 4CL promoters | | | | | | | Gaaa |
| SEQ_9 | P$REM/ATCTA.01 | Motif involved in carotenoid and toco- | 0.85 | 465 | 475 | − | 1.000 | 0.897 | caATCTataag |
| | | pherol biosynthesis and in the expression | | | | | | | |
| | | of photosynthesis-related genes | | | | | | | |
| SEQ_9 | P$GTBX/GT1.01 | GT1-Box binding factors with a trihelix | 0.85 | 471 | 487 | + | 0.968 | 0.852 | gattgtGTAAg |
| | | DNA-binding domain | | | | | | | tctata |
| SEQ_9 | P$MSAE/MSA.01 | M-phase-specific activators (NtmybA1, | 0.80 | 513 | 527 | + | 1.000 | 0.968 | agtccAACGgc |
| | | NtmybA2, NtmybB) | | | | | | | aaga |
| SEQ_9 | P$NCS2/NCS2.01 | Nodulin consensus sequence 2 | 0.79 | 538 | 552 | + | 1.000 | 0.795 | ggttgtCTCTg |
| | | | | | | | | | tgaa |
| SEQ_9 | P$LFYB/LFY.01 | Plant specific floral meristem identity | 0.93 | 580 | 592 | + | 0.914 | 0.948 | tCCCAatggta |
| | | gene LEAFY (LFY) | | | | | | | aa |
| SEQ_9 | P$MSAE/MSA.01 | M-phase-specific activators (NtmybA1, | 0.80 | 587 | 601 | + | 1.000 | 0.890 | ggtaaAACGgt |
| | | NtmybA2, NtmybB) | | | | | | | tgat |
| SEQ_9 | P$MYBL/MYBPH3.01 | Myb-like protein of Petunia hybrida | 0.80 | 588 | 604 | + | 0.750 | 0.831 | gtaaaacgGTT |
| | | | | | | | | | Gatgat |
| SEQ_9 | P$GBOX/ | bZIP transcription factor from | 0.76 | 604 | 624 | + | 1.000 | 0.804 | tggtggTGACa |
| | BZIP911.02 | Antirrhinum majus | | | | | | | tgtatgattg |
| SEQ_9 | P$OPAQ/O2.01 | Opaque-2 regulatory protein | 0.87 | 605 | 621 | − | 0.794 | 0.901 | tcatacatgTC |
| | | | | | | | | | ACcacc |
| SEQ_9 | P$OPAQ/ | Recognition site for BZIP transcription | 0.81 | 606 | 622 | + | 1.000 | 0.941 | gtggtgACATg |
| | O2_GCN4.01 | factors that belong to the group of | | | | | | | tatgat |
| | | Opaque-2 like proteins | | | | | | | |
| SEQ_9 | P$CAAT/CAAT.01 | CCAAT-box in plant promoters | 0.97 | 619 | 627 | − | 1.000 | 0.984 | aaCCAAtca |
| SEQ_9 | P$MIIG/P_ACT.01 | Maize activator P of flavonoid bio- | 0.93 | 695 | 709 | + | 0.966 | 0.969 | tggaGGTTggt |
| | | synthetic genes | | | | | | | cccg |
| SEQ_9 | P$IBOX/IBOX.01 | I-Box in rbcS genes and other light | 0.81 | 729 | 745 | + | 0.750 | 0.811 | ttgaaGAGAag |
| | | regulated genes | | | | | | | gttaag |
| SEQ_9 | P$CARM/CARICH.01 | CA-rich element | 0.78 | 739 | 757 | − | 1.000 | 0.803 | ggcctgcAACA |
| | | | | | | | | | tcttaacc |
| SEQ_9 | P$RAV5/RAV1-5.01 | 5′-part of bipartite RAV1 binding site, | 0.96 | 770 | 780 | − | 1.000 | 0.979 | tgcAACAcaaa |
| | | interacting with AP2 domain | | | | | | | |
| SEQ_9 | P$LEGB/RY.01 | RY and Sph motifs conserved in seed- | 0.87 | 782 | 808 | − | 1.000 | 0.895 | ggtgacttCAT |
| | | specific promoters | | | | | | | Gcaaaatctca |
| | | | | | | | | | gtctt |
| SEQ_9 | P$CCAF/CCA1.01 | Circadian clock associated 1 | 0.85 | 787 | 801 | − | 1.000 | 0.945 | tcatgcaaAAT |
| | | | | | | | | | Ctca |
| SEQ_9 | P$OCSE/OCSL.01 | OCS-like elements | 0.69 | 798 | 818 | − | 0.769 | 0.706 | caatcaaagag |
| | | | | | | | | | gtgACTTcat |
| SEQ_9 | P$LREM/ATCTA.01 | Motif involved in carotenoid and toco- | 0.85 | 824 | 834 | + | 1.000 | 0.916 | gcATCTaagaa |
| | | pherol biosynthesis and in the expression | | | | | | | |
| | | of photosynthesis-related genes | | | | | | | |
| SEQ_9 | O$RPOA/ | PolyA signal of D-type LTRs | 0.78 | 838 | 858 | + | 1.000 | 0.797 | gCCATtagaat |
| | DTYPEPA.01 | | | | | | | | gattgatttg |
| SEQ_9 | P$AHBP/ATHB5.01 | HDZip class I protein ATHB5 | 0.89 | 844 | 854 | + | 0.829 | 0.904 | agaATGAttga |
| SEQ_9 | P$AHBP/ATHB5.01 | HDZip class I protein ATHB5 | 0.89 | 844 | 854 | − | 0.936 | 0.977 | tcaATCAttct |
| SEQ_9 | P$LREM/ATCTA.01 | Motif involved in carotenoid and toco- | 0.85 | 857 | 867 | + | 1.000 | 0.854 | tgATCTacggt |
| | | pherol biosynthesis and in the expression | | | | | | | |
| | | of photosynthesis-related genes | | | | | | | |
| SEQ_9 | P$TELO/ATPURA.01 | Arabidopsis Telo-box interacting protein | 0.85 | 857 | 871 | − | 0.750 | 0.863 | tgaaACCGtag |
| | | related to the conserved animal protein | | | | | | | atca |
| | | Pur-alpha | | | | | | | |
| SEQ_9 | P$PSRE/GAAA.01 | GAAA motif involved in pollen specific | 0.83 | 859 | 875 | − | 1.000 | 0.843 | gcattGAAAcc |
| | | transcriptional activation | | | | | | | gtagat |
| SEQ_9 | P$GTBX/GT3A.01 | Trihelix DNA-binding factor GT-3a | 0.83 | 860 | 876 | + | 0.750 | 0.839 | tctacgGTTTc |
| | | | | | | | | | aatgcc |
| SEQ_9 | P$TELO/ATPURA.01 | Arabidopsis Telo-box interacting protein | 0.85 | 905 | 919 | − | 0.750 | 0.854 | aaaaATCCtaa |
| | | related to the conserved animal protein | | | | | | | gttg |
| | | Pur-alpha | | | | | | | |
| SEQ_9 | P$SPF1/SP8BF.01 | DNA-binding protein of sweet potato that | 0.87 | 917 | 929 | + | 1.000 | 0.872 | ttTACTttttc |
| | | binds to the SP8a (ACTGTGTA) and SP8b | | | | | | | tt |
| | | (TACTATT) sequences of sporamin and beta- | | | | | | | |
| | | amylase genes | | | | | | | |
| SEQ_9 | P$SPF1/SP8BF.01 | Motif involved in carotenoid and toco- | 0.85 | 936 | 946 | + | 1.000 | 0.859 | tgATCTactta |
| | | pherol biosynthesis and in the expression | | | | | | | |
| | | of photosynthesis-related genes | | | | | | | |
| SEQ_9 | O$LDPS/ | Lentiviral Poly A downstream element | 0.89 | 945 | 959 | + | 0.980 | 0.901 | taCTGTgtaat |
| | LDSPOLYA.01 | | | | | | | | tttt |
| SEQ_9 | P$L1BX/ATML1.01 | L1-specific homeodomain protein ATML1 | 0.82 | 961 | 977 | − | 1.000 | 0.838 | gccatcTAAAt |
| | | (A. thaliana meristem layer 1) | | | | | | | gaacca |
| SEQ_9 | P$LREM/ATCTA.01 | Motif involved in carotenoid and toco- | 0.85 | 966 | 976 | − | 1.00 | 0.960 | ccATCTaaatg |
| | | pherol biosynthesis and in the expression | | | | | | | |
| | | of photosynthesis-related genes | | | | | | | |
| SEQ_9 | P$MADS/AGL15.01 | AGL15, Arabidopsis MADS-domain protein | 0.79 | 976 | 996 | + | 0.925 | 0.796 | gctTTCTctca |
| | | AGAMOUS-like 15 | | | | | | | tatgaaactc |
| SEQ_9 | P$EINL/TEIL.01 | TEIL (tobacco EIN3-like) | 0.92 | 988 | 996 | + | 0.964 | 0.24 | aTGAAactc |
| SEQ_9 | P$CAAT/CAAT.01 | CCAAT-box in plant promoters | 0.97 | 1001 | 1009 | − | 1.000 | 0.983 | atCCAAtat |
| SEQ_9 | P$LREM/ATCTA.01 | Motif involved in carotenoid and toco- | 0.85 | 1017 | 1027 | + | 1.000 | 0.902 | taATCTataat |
| | | pherol biosynthesis and in the expression | | | | | | | |
| | | of photosynthesis-related genes | | | | | | | |
| SEQ_9 | P$GTBX/GT1.01 | GT1-Box binding factors with a trihelix | 0.85 | 1020 | 1036 | − | 1.000 | 0.866 | tattgtGTTAt |
| | | DNA-binding domain | | | | | | | tataga |
| SEQ_9 | P$IBOX/IBOX.01 | I-Box in rbcS genes and other light | 0.81 | 1034 | 1050 | + | 0.750 | 0.843 | ataaaAATAag |
| | | regulated genes | | | | | | | attact |
| SEQ_9 | P$SPF1/SP8BF.01 | DNA-binding protein of sweet potato that | 0.87 | 1045 | 1057 | + | 1.000 | 0.881 | atTACTgtcaa |
| | | binds to the SP8a (ACTGTGTA) and SP8b | | | | | | | tt |
| | | (TACTATT) sequences of sporamin and beta- | | | | | | | |
| | | amylase genes | | | | | | | |
| SEQ_9 | P$MYBL/ | Anther-specific myb gene from tobacco | 0.96 | 1065 | 1081 | + | 1.000 | 1.000 | ctaaggcaGTT |
| | NTMYBAS1.01 | | | | | | | | Aggtga |
| SEQ_9 | P$MIIG/PALBOXL.01 | Cis-acting element conserved in various | 0.80 | 1069 | 1083 | + | 1.000 | 0.827 | ggcagttaGGT |
| | | PAL and 4CL promoters | | | | | | | Gaca |
| SEQ_9 | P$AREF/ARE.01 | Auxin Response Element | 0.93 | 1074 | 1086 | − | 1.000 | 0.932 | ataTGTCacct |
| | | | | | | | | | aa |
| SEQ_9 | P$MADS/AGL2.01 | AGL2, Arabidopsis MADS-domain protein | 0.82 | 1091 | 1111 | − | 1.000 | 0.847 | ttcgaCCATag |
| | | AGAMOUS-like 2 | | | | | | | ctaggatcta |
| SEQ_9 | P$GARP/ARR10.01 | Type-B response regulator (ARR10), member | 0.97 | 1092 | 1100 | + | 1.000 | 0.970 | AGATcctag |
| | | of the GARP-family of plant myb-related | | | | | | | |
| | | DNA binding motifs | | | | | | | |
| SEQ_9 | P$L1BX/ATML1.01 | L1-specific homeodomain protein ATML1 | 0.82 | 1123 | 1139 | + | 0.750 | 0.834 | ttttatGAAAt |
| | | (A. thaliana meristem layer 1) | | | | | | | gtaggt |
| SEQ_9 | P$GTBX/GT1.01 | GT1-Box binding factors with a trihelix | 0.85 | 1132 | 1148 | + | 0.968 | 0.877 | atgtagGTAAg |
| | | DNA-binding domain | | | | | | | tattgg |
| SEQ_9 | P$CAAT/CAAT.01 | CCAAT-box in plant promoters | 0.97 | 1142 | 1150 | − | 1.000 | 0.977 | aaCCAAtac |
| SEQ_9 | P$GTBX/SBF1.01 | SBF-1 | 0.87 | 1183 | 1199 | + | 1.000 | 0.878 | tttgtgtTTAA |
| | | | | | | | | | tcaaat |
| SEQ_9 | P$AHBP/WUS.01 | Homeodomain protein WUSCHEL | 0.94 | 1186 | 1196 | + | 1.000 | 0.963 | gtgttTAATca |
| SEQ_9 | P$DOFF/PBOX.01 | Prolamin box, conserved in cereal seed | 0.75 | 1205 | 1221 | + | 0.761 | 0.793 | tgttctgaAAA |
| | | storage protein gene promoters | | | | | | | Attcaa |
| SEQ_9 | P$HEAT/HSE.01 | Heat shock element | 0.81 | 1206 | 1220 | − | 1.000 | 0.870 | tgaatttttcA |
| | | | | | | | | | GAAc |
| SEQ_9 | P$NCS1/NCS1.01 | Nadulin consensus sequence 1 | 0.85 | 1224 | 1234 | + | 1.000 | 0.966 | aAAAAgatcaa |
| SEQ_9 | P$MYBL/MYBPH3.02 | Myb-like protein of Petunia hybrida | 0.76 | 1234 | 1250 | + | 0.778 | 0.791 | aaaattTGGTt |
| | | | | | | | | | aaagaa |
| SEQ_9 | P$GTBX/GT1.01 | GT1-Box binding factors with a trihelix | 0.85 | 1236 | 1252 | + | 1.000 | 0.867 | aatttgGTTAa |
| | | DNA-binding domain | | | | | | | agaaga |
| SEQ_9 | P$DOFF/DOF1.01 | Doff/MNB1a-single zinc finger trans- | 0.98 | 1237 | 1253 | + | 1.000 | 0.981 | atttggttAAA |
| | | cription factor | | | | | | | Gaagac |
| SEQ_9 | P$OCSE/OCSL.01 | OCS-like elements | 0.69 | 1238 | 1258 | + | 0.807 | 0.720 | tttggttaaag |
| | | | | | | | | | aagACGAact |
| SE_9 | P$CCAF/CCA1.01 | Circadian clock associated 1 | 0.85 | 1257 | 1271 | + | 1.000 | 0.923 | cttacaaaAAT |
| | | | | | | | | | Cttg |
| SEQ_9 | P$MYBL/MYBPH3.02 | Myb-like protein of Petunia hybrida | 0.76 | 1274 | 1290 | + | 1.000 | 0.866 | ataactTAGTt |
| | | | | | | | | | aaatga |
| SEQ_9 | P$AHBP/ATHB9.01 | HD-ZIP class III protein ATHB9 | 0.77 | 1284 | 1294 | + | 1.000 | 0.757 | taaATGAtaac |
| SEQ_9 | P$IBOX/GATA.01 | Class I GATA factors | 0.93 | 1284 | 1300 | + | 1.000 | 0.963 | taaatGATAac |
| | | | | | | | | | aaatct |
| SEQ_9 | P$NCS1/NCS1.01 | Nadulin consensus sequence 1 | 0.85 | 1284 | 1294 | + | 0.878 | 0.909 | tAAATgataac |
| SEQ_9 | P$MYBL/GAMYB.01 | GA-regulated myb gene from barley | 0.91 | 1286 | 1302 | − | 1.000 | 0.949 | gcagatttGTT |
| | | | | | | | | | Atcatt |
| SEQ_9 | P$CCAF/CCA1.01 | Circadian clock associated 1 | 0.85 | 1288 | 1302 | + | 1.000 | 0.861 | tgataacaAAT |
| | | | | | | | | | Ctgc |
| SEQ_9 | P$OCSE/OCSL.01 | OCS-like elements | 0.69 | 1305 | 1325 | + | 0.807 | 0.701 | gtcaataaaaa |
| | | | | | | | | | gttACGAgtc |
| SEQ_9 | P$MYBL/MYBPH3.01 | Myb-like protein of Petunia hybrida | 0.80 | 1308 | 1324 | + | 1.000 | 0.810 | aataaaaaGTT |
| | | | | | | | | | Acgagt |
| SEQ_9 | P$GTGX/GT3A.01 | Trihelix DNA-binding factor GT-3a | 0.83 | 1310 | 1326 | + | 1.000 | 0.876 | taaaaaGTTAc |
| | | | | | | | | | gagtct |
| SEQ_9 | P$MYBL/MYBPH3.02 | Myb-like protein of Petunia hybrida | 0.76 | 1372 | 1388 | + | 1.000 | 0.926 | acaagtTAGTt |
| | | | | | | | | | gagtca |
| SEQ_9 | P$OPAQ/GCN4.01 | GCN4, conserved in cereal seed storage | 0.81 | 1377 | 1393 | + | 1.000 | 0.880 | ttagtTGAGtc |
| | | protein gene promoters, similar to yeast | | | | | | | acgtgc |
| | | GCN4 and vertebrate AP-1 | | | | | | | |
| SEQ_9 | P$GBOX/CPRF.01 | Common plant regulatory factor (CPRF) | 0.95 | 1379 | 1399 | − | 1.000 | 0.975 | aggtaagcACG |
| | | from parsley | | | | | | | Tgactcaact |
| SEQ_9 | P$GBOX/CPRF.01 | Common plant regulatory factor (CPRF) | 0.95 | 1380 | 1400 | + | 1.000 | 0.966 | gttgagtcACG |
| | | from parsley | | | | | | | Tgcttacctt |
| SEQ_9 | P$MCL/ | Rice bHLH protein | 0.85 | 1380 | 1398 | − | 1.000 | 0.933 | ggtaagCACGt |
| | OSBHLH66.01 | | | | | | | | gactcaac |
| SEQ_9 | P$MYCL/MY CRS.01 | Myc recognition sequences | 0.93 | 1381 | 1399 | + | 1.000 | 0.990 | ttgagtcACGT |
| | | | | | | | | | gcttacct |
| SEQ_9 | P$OPAQ/RITA1.01 | Rice transcription activator-1 (RITA), | 0.95 | 1381 | 1397 | − | 1.000 | 0.970 | gtaagcACGTg |
| | | basic leucin zipper protein, highly | | | | | | | actcaa |
| | | expressed during seed development | | | | | | | |
| SEQ_9 | P$ABRE/ABRE.01 | ABA response elements | 0.82 | 1382 | 1398 | − | 1.000 | 0.832 | ggtaagcACGT |
| | | | | | | | | | gactca |
| SEQ_9 | O$RPOA/APOLYA.01 | Avian C-type LTR PolyA signal | 0.71 | 1397 | 1417 | + | 1.000 | 0.797 | ccttcTAAAaa |
| | | | | | | | | | gcctttttga |
| SEQ_9 | P$DOFF/DOF3.01 | Dof3-single zinc finger transcription | 0.99 | 1397 | 1413 | + | 1.000 | 0.969 | ccttctaaAAA |
| | | factor | | | | | | | Gccttt |
| SEQ_9 | O$RPOA/APOLYA.01 | Avian C-type LTR PolyA signal | 0.71 | 1401 | 1421 | − | 0.750 | 0.720 | ttgatCAAAaa |
| | | | | | | | | | ggctttttag |
| SEQ_9 | P$LFYB/LFY.01 | Plant specific floral meristem identity | 0.93 | 1414 | 1426 | − | 0.914 | 0.938 | aACCAttgatc |
| | | gene LEAFY (LFY) | | | | | | | aa |
| SEQ_9 | P$SBPD/SBP.01 | SQUA promoter binding proteins | 0.88 | 1419 | 1435 | − | 1.000 | 0.895 | atttgGTACaa |
| | | | | | | | | | ccattg |
| SEQ_9 | P$SBPD/SBP.01 | SQUA promoter binding proteins | 0.88 | 1422 | 1438 | + | 1.000 | 0.902 | tggttGTACca |
| | | | | | | | | | aatgag |
| SEQ_9 | P$MADS/AG.01 | Agamous, required for normal flower | 0.80 | 1425 | 1445 | + | 1.000 | 0.808 | ttgTACCaaat |
| | | development, similarity to SRF (human) | | | | | | | gagaagagag |
| | | and MCM (yeast) proteins | | | | | | | |
| SEQ_9 | P$GAGA/BPC.01 | Basic pentacysteine proteins | 1.00 | 1436 | 1460 | + | 1.000 | 1.000 | gagaagAGAGa |
| | | | | | | | | | ctataagcgtt |
| | | | | | | | | | gca |
| SEQ_9 | P$SPF1/SP8BF.01 | DNA-binding protein of sweet potato that | 0.87 | 1464 | 1476 | + | 1.000 | 0.872 | ttTACTtttac |
| | | binds to the SP8a (ACTGTGTA) and SP8b | | | | | | | tt |
| | | (TACTATT) sequences of sporamin and beta- | | | | | | | |
| | | amylase genes | | | | | | | |
| SEQ_9 | P$SPF1/SP8BF.01 | DNA-binding protein of sweet potato that | 0.87 | 1470 | 1482 | + | 1.000 | 0.872 | ttTACTtttac |
| | | binds to the SP8a (ACTGTGTA) and SP8b | | | | | | | tt |
| | | (TACTATT) sequences of sporamin and beta- | | | | | | | |
| | | amylase genes | | | | | | | |
| SEQ_9 | P$TBPF/TATA.02 | Plant TATA box | 0.90 | 1481 | 1495 | + | 1.000 | 0.940 | ttccTATAtaa |
| | | | | | | | | | aaag |
| SEQ_9 | P$TBPF/TATA.01 | Plant TATA box | 0.88 | 1483 | 1497 | + | 1.000 | 0.954 | cctaTATAaaa |
| | | | | | | | | | agtc |
| SEQ_9 | P$OCSE/OCSL.01 | OCS-like elements | 0.69 | 1500 | 1520 | − | 1.000 | 0.708 | tgttatgaggt |
| | | | | | | | | | ttaACGTctt |
| SEQ_9 | P$MYBL/GAMYB.01 | GA-regulated myb gene from barley | 0.91 | 1511 | 1527 | − | 1.000 | 0.954 | atttgtttGTT |
| | | | | | | | | | Atgagg |
| SEQ_9 | P$TBPF/TATA.02 | Plant TATA box | 0.90 | 1523 | 1537 | + | 1.000 | 0.914 | caaaTATAaat |
| | | | | | | | | | ttct |
| SEQ_9 | P$TBPF/TATA.02 | Plant TATA box | 0.90 | 1545 | 1559 | − | 1.000 | 0.911 | atatTATAaat |
| | | | | | | | | | tctt |

TABLE 4
cis-regulatory elements of SEQ ID NO: 10

| | | | | | | | | | |
|---|---|---|---|---|---|---|---|---|---|
| SEQ_10 | P$PSRE/GAAA.01 | GAAA motif involved in pollen specific | 0.83 | 3 | 191 | + | 1.000 | 0.881 | tgatgGAAAtcgt |
| | | transcriptional activation | | | | | | | atcg |
| SEQ_10 | P$MIIG/P_ACT.01 | Maize activator P of flavonoid bio- | 0.93 | 99 | 13 | + | 0.966 | 0.983 | agctGGTTggtac |
| | | synthetic genes | | | | | | | ga |
| SEQ_10 | P$SBPD/SBP.01 | SQUA promoter binding proteins | 0.88 | 001 | 161 | − | 1.000 | 0.914 | ttatcGTACcaa |
| | | | | | | | | | ccagc |
| SEQ_10 | P$1BOX/GATA.01 | Class I GATA factors | 0.93 | 071 | 231 | + | 1.000 | 0.958 | ggtacGATAataa |
| | | | | | | | | | tgta |
| SEQ_10 | P$AHBP/HAHB4.01 | Sunflower homeodomain leucine-zipper | 0.87 | 131 | 231 | − | 1.000 | 0.909 | tacattATTAt |
| | | protein Hahb-4 | | | | | | | |
| SEQ_10 | P$OPAQ/ | Recognition site for BZIP transcription | 0.81 | 271 | 431 | + | 0.829 | 0.818 | tgcatcACCTgct |
| | O2_GCN4.01 | factors that belong to the group of | | | | | | | taaa |
| | | Opaque-like proteins | | | | | | | |
| SEQ_10 | P$STKM/STK.01 | Storekeeper (STK), plant specific DNA | 0.85 | 391 | 531 | − | 0.833 | 0.863 | accCAAActattt |
| | | binding protein important for tuber | | | | | | | aa |
| | | specific and sucrose-inducible gene | | | | | | | |
| | | expression | | | | | | | |
| SEQ_10 | P$CARM/CARICH.01 | CA-rich element | 0.78 | 451 | 631 | − | 1.000 | 0.832 | tgaaacaAACAcc |
| | | | | | | | | | caaact |
| SEQ_10 | P$AREF/ARE.01 | Auxin Response Element | 0,93 | 821 | 942 | + | 1.000 | 0.951 | ttaTGTCtcagtt |
| SEQ_10 | P$MYBL/GAMYB.01 | GA-regulated myb gene from barley | 0.91 | 842 | 002 | + | 1.000 | 0.915 | atgtctcaGTTAt |
| | | | | | | | | | atct |
| SEQ_10 | P$OPAQ/ | Recognition site for BZIP transcription | 0.81 | 012 | 172 | − | 0.829 | 0.821 | gaaattACTTggt |
| | O2_GCN4.01 | factors that belong to the group of | | | | | | | ttac |
| | | Opaque-like proteins | | | | | | | |
| SEQ_10 | P$CARM/CARICH.01 | CA-rich element | 0.78 | 122 | 302 | − | 1.000 | 0.790 | gtgaacgAACAcc |
| | | | | | | | | | gaaatt |
| SEQ_10 | P$GBOX/HBP1B.01 | Wheat bZIP transcription factor HBP1B | 0.83 | 342 | 542 | − | 1.000 | 0.834 | actttgggACGTa |
| | | (histone gene binding protein 1b) | | | | | | | agtaatat |
| SEQ_10 | P$DOFF/DOF2.01 | Dof-single zinc finger transcription | 0.98 | 562 | 722 | + | 1.000 | 0.995 | atatctatAAAGc |
| | | factor | | | | | | | aagt |
| SEQ_10 | P$LREM/ATCTA.01 | Motif involved in carotenoid and toco- | 0.85 | 562 | 662 | + | 1.000 | 0.922 | atATCTataaa |
| | | pherol biosynthesis and in the expression | | | | | | | |
| | | of photosynthesis-related genes | | | | | | | |
| SEQ_10 | P$TBPF/TATA.01 | Plant TATA box | 0.88 | 572 | 712 | + | 1.000 | 0.882 | tatcTATAaagca |
| | | | | | | | | | ag |
| SEQ_10 | P$OPAQ/ | Recognition site for BZIP transcription | 0.81 | 622 | 782 | − | 0.829 | 0.846 | cagatcACTTgct |
| | O2_GCN4.01 | factors that belong to the group of | | | | | | | ttat |
| | | Opaque-like proteins | | | | | | | |
| SEQ_10 | O$RPAD/PADS.01 | Mammalian C-type LTR Poly A downstream | 0.87 | 68 | 80 | + | 0.883 | 0.876 | caaGTGAtctgat |
| | | element | | | | | | | |
| SEQ_10 | P$MYBS/HVMCB1.0 | Hordeum vulgare Myb-related CAB-promoter- | 0.93 | 752 | 912 | + | 1.000 | 0.952 | tctgatATCCtat |
| | | binding protein | | | | | | | gaaa |
| SEQ_10 | P$GAPB/GAP.01 | Cis-element in the GAPDH promoters | 0.88 | 823 | 963 | + | 1.000 | 0.917 | tcctATGAaaatc |
| | | conferring light inducibility | | | | | | | aa |
| SEQ_10 | P$GTBX/GT1.0 | GT1-Box binding factors with a trihelix | 0.85 | 353 | 513 | + | 1.000 | 0.855 | cagaggGTTAatt |
| | | DNA-binding domain | | | | | | | aaaa |
| SEQ_10 | P$TELO/ATPURA.01 | Arabidopsis Telo-box interacting protein | 0.85 | 474 | 614 | + | 0.750 | 0.868 | taaaACGCtaaat |
| | | related to the conserved animal protein | | | | | | | ca |
| | | Pur-alpha | | | | | | | |
| SEQ_10 | P$TBPF/TATA.01 | Plant TATA box | 0,88 | 324 | 464 | − | 1.000 | 0.985 | ttcaTATAaatat |
| | | | | | | | | | at |
| SEQ_10 | P$AHBP/ATHB5.01 | HDZip class I protein ATHB5 | 0.89 | 434 | 534 | + | 0.829 | 0.904 | tgaATGAttga |
| SEQ_10 | P$AHBP/ATHB5.01 | HDZip class I protein ATHB3 | 0.89 | 434 | 534 | − | 0.936 | 0.977 | tcaATCAttca |
| SEQ_10 | P$AHBP/WUS.01 | Homeodomain protein WUSCHEL | 0.94 | 514 | 614 | − | 1.000 | 0.963 | tctttTAATca |
| SEQ_10 | P$PSRE/GAAA.01 | GAAA motif involved in pollen specific | 0.83 | 554 | 714 | + | 1.000 | 0.876 | taaaaGAAAtgtt |
| | | transcriptional activation | | | | | | | caaa |
| SEQ_10 | P$MYBL/MYBPH3.01 | Myb-like protein of Petunia hybrida | 0.80 | 724 | 885 | − | 0.750 | 0.833 | aagaaacgCTTAt |
| | | | | | | | | | acat |
| SEQ_10 | P$PSRE/GAAA.01 | GAAA motif involved in pollen specific | 0.83 | 88 | 04 | + | 1.000 | 0.837 | tctgaGAAAattt |
| | | transcriptional activation | | | | | | | acaa |
| SEQ_10 | P$GTBX/GT1.0 | GT1-Box binding factors with a trihelix | 0.85 | 492 | 408 | − | 0.968 | 0.891 | aattttGTAAatt |
| | | DNA-binding domain | | | | | | | ttct |
| SEQ_10 | P$OPAQ/ | Recognition site for BZIP transcription | 0.81 | 155 | 315 | − | 1.000 | 0.855 | ctgtgtACATggc |
| | O2_GCNr.01 | factors that belong to the group of | | | | | | | taaa |
| | | Opaque-like proteins | | | | | | | |
| SEQ_10 | P$EREF/ANT.01 | ANT (Arabidopsis protein AINTEGUMEN), | 0.81 | 415 | 575 | − | 1.000 | 0.820 | agaatatTCCCaa |
| | | member of the plant-specific family of | | | | | | | tgct |
| | | AP2/EREBP-transcription factors | | | | | | | |
| SEQ_10 | P$MYBS/ | MYB protein from wheat | 0.83 | 425 | 585 | − | 1.000 | 0.987 | gagaATATtccca |
| | | TAMYB80.01 | | | | | | | atgc |

| | | | | | | | | | |
|---|---|---|---|---|---|---|---|---|---|
| SEQ_10 | P$MYBS/ | MYB protein from wheat | 0.83 | 475 | 635 | + | 1.000 | 0.941 | gggaATATtctct |
| | TAMYB8001 | | | | | | | | tcac |
| SEQ_10 | P$HEAT/HSE.01 | Heat shock element | 0.81 | 675 | 815 | − | 1.000 | 0.835 | tgaaagaaaaAGA |
| | | | | | | | | | At |
| SEQ_10 | P$IBOX/GATA.01 | Class I factors | 0.93 | 785 | 946 | − | 1.000 | 0.947 | gcataGATAatgt |
| | | | | | | | | | tgaa |
| SEQ_10 | P$DOFF/DOF3.01 | Dof3-single zinc finger transcription | 0.99 | 895 | 056 | − | 1.000 | 0.997 | aaatttcaAAAGc |
| | | factor | | | | | | | atag |
| SEQ_10 | P$PREM/ | Promoter elements involved in MgProto | 0.77 | 925 | 226 | − | 1.000 | 0.778 | ccatCGACaacaa |
| | MGPROTORE.01 | (Mg-protoporphyrin IX) and light-mediated | | | | | | | gttaaaatttcaa |
| | | induction | | | | | | | aagca |
| SEQ_10 | P$PSRE/GAAA.01 | GAAA motif involved in pollen specific | 0.83 | 946 | 106 | + | 1.000 | 0.837 | cttttGAAAtttt |
| | | transcriptional activation | | | | | | | aact |
| SEQ_10 | P$IDDF/ID1.01 | Maize INDETERMINATE1 zinc finger protein | 0.92 | 096 | 216 | + | 1.000 | 0.939 | cttgTTGTcgatg |
| SEQ_10 | P$MYBS/ | Hordeum vulgare Myb-related CAB-promoter- | 0.93 | 146 | 306 | − | 1.000 | 0.945 | aagcatATCCatc |
| | HVMCB1.01 | binding protein | | | | | | | gaca |
| SEQ_10 | P$MYBS/ | MYB protein from wheat | 0.83 | 196 | 356 | + | 1.000 | 0.907 | atggATATgctta |
| | TAMYB80.01 | | | | | | | | gaga |
| SEQ_10 | P$PSRE/GAAA.01 | GAAA motif involved in pollen specific | 0.83 | 296 | 456 | + | 1.000 | 0.956 | ttagaGAAAtttt |
| | | transcriptional activation | | | | | | | caaa |
| SEQ_10 | P$AHBP/WUS.01 | Homeodomain protein WUSCHEL | 0.94 | 636 | 736 | + | 1.000 | 0.963 | aacatTAATca |
| SEQ_10 | P$AHBP/WUS.01 | Homeodomain protein WUSCHEL | 0.94 | 646 | 746 | − | 1.000 | 1.000 | ttgatTAATgt |
| SEQ_10 | P$1BOX/GATA.01 | Class I factors | 0.93 | 706 | 867 | + | 1.000 | 0.971 | atcaaGATAagct |
| | | | | | | | | | agca |
| SEQ_10 | P$PALA/ | Putative cis-acting element on various | 0.84 | 987 | 167 | + | 0.825 | 0.588 | tgactgtCCATcc |
| | PALBOXA.01 | PAL and 4CL gene promoters | | | | | | | aatata |
| SEQ_10 | P$CAAT/CAAT.01 | CCAAT-box in plant promoters | 0.97 | 077 | 157 | + | 1.000 | 0.983 | atCCAAtat |
| SEQ_10 | O$RPOA/APOLYA.01 | Avian C-type LTR PolyA signal | 0.71 | 407 | 607 | + | 0.750 | 0.765 | tataaTATAtcga |
| | | | | | | | | | caattttt |
| SEQ_10 | P$GTBX/SBF1.01 | SBF | 0.87 | 537 | 697 | − | 1.000 | 0.885 | cgttttcTTAAaa |
| | | | | | | | | | attg |
| SEQ_10 | P$MSAE/MSA.01 | M-phase-specific activators (NtmybA1, | 0.80 | 617 | 757 | + | 1.000 | 0.871 | aagaaAACGgtat |
| | | NtmybA2, NtmybB) | | | | | | | aa |
| SEQ_10 | O$RVUP/LTRUP.01 | Upstream element of C-type Long Terminal | 0.76 | 66 | 86 | + | 1.000 | 0.809 | aacggtataacTT |
| | | Repeats | | | | | | | TCaaagct |
| SEQ_10 | P$L1BX/ATML1.01 | L1-specific homeodomain protein ATML1 | 0.82 | 791 | 807 | + | 1.000 | 0.830 | ggtttaTAAAtgt |
| | | (A. thaliana meristem layer 1) | | | | | | | caaa |
| SEQ_10 | P$TBPF/TATA.02 | Plant A box | 0.90 | 917 | 058 | + | 1.000 | 0.914 | ggttTATAaatgt |
| | | | | | | | | | ca |
| SEQ_10 | P$OPAQ/ | Recognition site for BZIP transcription | 0.81 | 937 | 098 | − | 1.000 | 0.858 | cctttgACATtta |
| | O2_GCN4.01 | factors that belong to the group of | | | | | | | taaa |
| | | Opaque-like proteins | | | | | | | |
| SEQ_10 | P$WBXF/WRKY.01 | WRKY plant specific zinc-finger-type | 0.92 | 958 | 118 | − | 1.000 | 0.936 | gtcctTTGAcatt |
| | | factor associated with pathogen defence, | | | | | | | tata |
| | | W box | | | | | | | |
| SEQ_10 | P$MYBL/MYBPH3.01 | Myb-like protein of Petunia hybrida | 0.80 | 188 | 348 | + | 1.000 | 0.908 | tcaaacccGTTAg |
| | | | | | | | | | tcaa |
| SEQ_10 | P$MSAE/MSA.01 | M-phase-specific activators (NtmybA1, | 0.80 | 198 | 338 | − | 1.000 | 0.854 | tgactAACGggtt |
| | | NtmybA2, NtmybB) | | | | | | | tg |
| SEQ_10 | P$MYBL/MYBPH3.02 | Myb-like protein of Petunia hybrida | 0.76 | 228 | 388 | + | 1.000 | 0.764 | acccgtTAGTcaa |
| | | | | | | | | | ggtt |
| SEQ_10 | P$OCSE/OCSL.01 | OCS-like elements | 0.69 | 508 | 708 | + | 0.769 | 0.705 | atacacaagcact |
| | | | | | | | | | cACCTact |
| SEQ_10 | P$MIIG/ | Cis-acting element conserved in various | 0.80 | 608 | 748 | − | 1.000 | 0.851 | gtgtagtaGGTGa |
| | PALBOXL.01 | PAL and 4CL promoters | | | | | | | gt |
| SEQ_10 | P$DREB/ | C-repeat/dehydration response element | 0.89 | 698 | 838 | + | 1.000 | 0.923 | ctacaCCGAcact |
| | CRT_DRE.01 | | | | | | | | ga |
| SEQ_10 | P$OCSE/OCSTF.01 | bZIP transcription factor binding to | 0.73 | 75 | 95 | + | 1.000 | 0.773 | cgacactgacatt |
| | | OCS-elements | | | | | | | GACGtctc |
| SEQ_10 | P$GBOX/HBP1B.01 | Wheat bZIP transcription factor HBP1B | 0.83 | 880 | 900 | − | 1.000 | 0.891 | aatcagagACGTc |
| | | (histone gene binding protein 1b) | | | | | | | aatgtcag |
| SEQ_10 | P$GBOX/TGA1.01 | Arabidopsis leucine zipper protein TGA1 | 0.90 | 818 | 019 | + | 1.000 | 0.953 | tgacatTGACgtc |
| | | | | | | | | | tctgattc |
| SEQ_10 | P$MADS/SQUA.01 | MADS-box protein SQUAMOSA | 0.90 | 958 | 159 | − | 1.000 | 0.904 | gagtccaATTTat |
| | | | | | | | | | agaatcag |
| SEQ_10 | P$MADS/L15.01 | AGL15, Arabidopsis MADS-domain protein | 0.79 | 968 | 169 | + | 0.925 | 0.873 | tgaTTCTataaat |
| | | AGAMOUS-like 15 | | | | | | | tggactca |
| SEQ_10 | P$TBPF/TATA.02 | Plant A box | 0.90 | 989 | 129 | + | 1.000 | 0.937 | attcTATAaattg |
| | | | | | | | | | ga |
| SEQ_10 | P$DOFF/DOF1.01 | Dof/MNB1a-single zinc finger | 0.98 | 169 | 329 | + | 1.000 | 0.984 | agattcctAAAGa |
| | | transcription factor | | | | | | | cgag |
| SEQ_10 | P$MYBL/GAMYB.01 | GA-regulated myb gene from barley | 0.91 | 309 | 469 | − | 1.000 | 0.929 | atgtatttGTTAt |
| | | | | | | | | | gctc |
| SEQ_10 | P$DOFF/PBOX.01 | Prolamin box, conserved in cereal seed | 0.75 | 509 | 669 | + | 1.000 | 0.843 | agcactgcAAAGa |
| | | storage protein gene promoters | | | | | | | taaa |
| SEQ_10 | P$IBOX/GATA.01 | Class I factors | 0.93 | 569 | 729 | + | 1.000 | 0.975 | gcaaaGATAaaaa |
| | | | | | | | | | aaaa |
| SEQ_10 | P$DOFF/PBF.01 | PBF (MPBF) | 0.97 | 62 | 78 | + | 1.000 | 0.979 | ataaaaaaAAAGg |
| | | | | | | | | | ggta |

TABLE 5
cis-regulatory elements of SEQ ID NO: 11

| | | | | | | | | | |
|---|---|---|---|---|---|---|---|---|---|
| SEQ_11 | P$AHBP/WUS.01 | Homeodomain protein WUSCHEL | 0.94 | 9 | 19 | + | 1.000 | 1.000 | tccctTAATgg |
| SEQ_11 | P$GTBX/SBF1.01 | SBF-1 | 0.87 | 15 | 32 | + | 1.000 | 0.886 | atggagcTTAAac |
| | | | | | | | | | tctt |
| SEQ_11 | P$L1BX/ATML1.01 | L1-specific homeodomain protein ATML | 0.82 | 331 | 47 | − | 1.000 | 0.835 | ttgtatTAAAttc |
| | | (A. thaliana meristem layer 1) | | | | | | | agaa |
| SEQ_11 | P$MYBL/MYBOH3.02 | Myb-like protein of Petunia hybrida | 0.76 | 44 | 60 | + | 0.778 | 0.832 | acaagtTGGTttg |
| | | | | | | | | | atca |
| SEQ_11 | P$WBXF/ERE.01 | Elicitor response element | 0.89 | 91 | 107 | − | 1.000 | 0.903 | ttattcTGACcat |
| | | | | | | | | | tgta |
| SEQ_11 | P$MYBL/GAMYB.01 | GA-regulated myb gene from barley | 0.91 | 100 | 116 | − | 1.000 | 0.919 | ttttctttGTTAt |
| | | | | | | | | | tctg |
| SEQ_11 | P$GTBX/GT1.01 | GT1-Box binding factors with a tri- | 0.85 | 110 | 126 | − | 0.968 | 0.858 | attgtaGTAAttt |
| | | helix DNA-binding domain | | | | | | | tctt |
| SEQ_11 | P$NCS1/NCS1.01 | Nodulin consensus sequence 1 | 0.85 | 135 | 145 | + | 0.804 | 0.882 | aAAACgatttg |
| SEQ_11 | P$GBOX/UPRE.01 | UPRE (unfolded protein response | 0.86 | 137 | 157 | − | 1.000 | 0.950 | tacttaCCACgtc |
| | | element) like motif | | | | | | | aaatcgtt |
| SEQ_11 | P$GBOX/GBF1.01 | bZIP protein G-Box binding factor 1 | 0.94 | 138 | 158 | + | 1.000 | 0.987 | acgatttgACGTg |
| | | | | | | | | | gtaagtat |
| SEQ_11 | P$WBXF/WRKY.01 | WRKY plant specific zinc-finger-type | 0.92 | 138 | 154 | + | 1.000 | 0.937 | acgatTTGAcgtg |
| | | factor associated with pathogen defence, | | | | | | | gtaa |
| | | W box | | | | | | | |
| SEQ_11 | P$ABRE/ABRE.01 | ABA response elements | 0.82 | 139 | 155 | + | 1.000 | 0.875 | cgatttgACGTgg |
| | | | | | | | | | taag |
| SEQ_11 | P$OPAQ/O2.02 | Opaque-regulatory protein | 0.87 | 139 | 155 | − | 1.000 | 0.909 | cttaCCACgtcaa |
| | | | | | | | | | atcg |
| SEQ_11 | P$GAPB/GAP.01 | Cis-element in the GAPDH promoters | 0.88 | 154 | 168 | − | 1.000 | 0.881 | gccaATGAaaata |
| | | conferring light inducibility | | | | | | | ct |
| SEQ_11 | P$CAAT/CAAT.01 | CCAAT-box in plant promoters | 0.97 | 161 | 169 | − | 1.000 | 0.978 | agCCAAtga |
| SEQ_11 | P$GBOX/GBF1.01 | . bZIP protein G-Box binding factor 1 | 0.94 | 170 | 190 | − | 1.000 | 0.967 | cttgttatACGTg |
| | | | | | | | | | tgagaact |
| SEQ_11 | P$ABRE/ABRE.01 | ABA response elements | 0.82 | 173 | 189 | − | 1.000 | 0.880 | ttgttatACGTgt |
| | | | | | | | | | gaga |
| SEQ_11 | P$AHBP/HAHB4.01 | Sunflower homeodomain leucine-zipper | 0.87 | 192 | 202 | − | 1.000 | 0.902 | catataATTAg |
| | | protein Hahb-4 | | | | | | | |
| SEQ_11 | O$LTUP/TAACC.01 | Lentiviral TATA upstream element | 0.81 | 262 | 284 | − | 1.000 | 0.722 | tactctaagtccA |
| | | | | | | | | | ACCcaaacag |
| SEQ_11 | P$CGCG/ATSR1.01 | Arabidopsis thaliana signal-responsive | 0.84 | 296 | 312 | − | 1.000 | 0.941 | cccCGCGtaattt |
| | | gene1, Ca2+/ calmodulin binding | | | | | | | ccga |
| | | protein homolog to NtER1 (tobacco | | | | | | | |
| | | early ethylene-responsive gene) | | | | | | | |
| SEQ_11 | P$AHBP/WUS.01 | Homeodomain protein WUSCHEL | 0.94 | 311 | 321 | − | 1.000 | 0.963 | ccaatTAATcc |
| SEQ_11 | P$CAAT/CAAT.01 | CCAAT-box in plant promoters | 0.97 | 315 | 323 | − | 1.000 | 0.976 | ccCCAAtta |
| SEQ_11 | O$RPOA/POLYA.01 | Mammalian C-type LTR Poly A signal | 0.76 | 318 | 338 | − | 1.000 | 0.807 | tgcaaTAAAacta |
| | | | | | | | | | atccccaa |
| SEQ_11 | P$L1BX/ATML1.01 | L1-specific homeodomain protein ATML | 0.82 | 332 | 348 | − | 0.750 | 0.855 | tagttaTATAtgc |
| | | (A. thaliana meristem layer 1) | | | | | | | aata |
| SEQ_11 | O$RPOA/ | PolyA signal of D-type LTRs | 0.78 | 337 | 357 | − | 0.750 | 0.852 | aCCCTtaaatagt |
| | DTYPEPA.01 | | | | | | | | tatatatg |
| SEQ_11 | P$MYBL/MYBPH3.02 | Myb-like protein of Petunia hybrida | 0.76 | 338 | 354 | − | 1.000 | 0.773 | cttaaaTAGTtat |
| | | | | | | | | | atat |
| SEQ_11 | P$MADS/AGL3.02 | AGL3, MADS Box protein | 0.80 | 340 | 360 | − | 0.790 | 0.853 | ataacCCTTaaat |
| | | | | | | | | | agttatat |
| SEQ_11 | P$SPF1/SP8B | DNA-binding protein of sweet potato | 0.87 | 342 | 354 | + | 0.777 | 0.893 | atAACTatttaag |
| | | that binds to the SP8a (ACTGTGTA) and | | | | | | | |
| | | SP8b (TACTATT) sequences of sporamin | | | | | | | |
| | | and beta-amylase genes | | | | | | | |
| SEQ_11 | P$TEFB/TEF1.01 | TEF cis acting elements in both RNA | 0.76 | 351 | 371 | + | 1.000 | 0.778 | taAGGGttatata |
| | | polymerase II-dependent promoters and | | | | | | | ggacatat |
| | | rDNA spacer sequences | | | | | | | |
| SEQ_11 | P$OCSE/OCSL.01 | OCS-like elements | 0.69 | 352 | 372 | + | 0.807 | 0.691 | aagggttatatag |
| | | | | | | | | | gACATatg |
| SEQ_11 | P$TBPF/TATA.02 | Plant A box | 0.90 | 353 | 367 | − | 1.000 | 0.903 | gtccTATAtaacc |
| | | | | | | | | | ct |
| SEQ_11 | P$MYCL/ICE..01 | ICE (inducer of CBF expression 1), AtMYC2 | 0.95 | 360 | 378 | − | 1.000 | 0.959 | gtttcACATatgt |
| | | (rd22BP1) | | | | | | | cctata |
| SEQ_11 | P$MYBL/MYBPH3.02 | Myb-like protein of Petunia hybrida | 0.76 | 375 | 391 | − | 1.000 | 0.819 | tatcgtTAGTtta |
| | | | | | | | | | gttt |
| SEQ_11 | P$IBOX/GATA.01 | Class I factors | 0.93 | 383 | 399 | + | 1.000 | 0.944 | ctaacGATAattc |
| | | | | | | | | | gtgg |
| SEQ_11 | O$RPAD/PADS.01 | Mammalian C-type LTR Poly A downstream | 0.87 | 393 | 405 | + | 1.000 | 0.895 | ttcGTGGtctagt |
| | | element | | | | | | | |
| SEQ_11 | P$CE3S/CE3.01 | Coupling element 3 (CE3), non-ACGT ABRE | 0.77 | 428 | 446 | − | 0.750 | 0.800 | ttgtaaCCCGtgt |
| | | | | | | | | | cctatg |
| SEQ_11 | P$NCS1/NCS1.01 | Nodulin consensus sequence 1 | 0.85 | 464 | 474 | − | 1.000 | 0.858 | aAAAAgttgaa |
| SEQ_11 | P$MYBL/MYBPH3.02 | Myb-like protein of Petunia hybrida | 0.76 | 475 | 491 | − | 1.000 | 0.872 | taaactTAGTtaa |
| | | | | | | | | | aaaa |
| SEQ_11 | P$DOFF/DOF2.01 | Dof-single zinc finger transcription | 0.98 | 487 | 503 | + | 1.000 | 1.000 | gtttaattAAAGc |
| | | factor | | | | | | | aaaa |
| SEQ_10 | P$IDDF/ID1.01 | Maize INDETERMINATE1 zinc finger | 0.92 | 501 | 513 | − | 1.000 | 0.963 | atttTTGTcattt |
| | | protein | | | | | | | |
| SEQ_11 | P$NCS1/NCS1.01 | Nadulin consensus sequence 1 | 0.85 | 512 | 522 | − | 1.000 | 0.855 | gAAAAgaatat |
| SEQ_11 | P$DOFF/DOF3.01 | Dof-single zinc finger transcription | 0.99 | 549 | 565 | − | 1.000 | 0.998 | aaagtgaaAAAGc |
| | | factor | | | | | | | ggcc |
| SEQ_11 | P$HEAT/HSE.01 | Heat shock element | 0.81 | 575 | 589 | − | 1.000 | 0.857 | aaaaacatcaAGA |
| | | | | | | | | | Aa |
| SEQ_11 | P$GBOX/ | bZIP transcription factor from | 0.76 | 595 | 615 | + | 0.750 | 0.783 | ataagtTGATgtg |
| | BZIP911.02 | Antirrhinum majus | | | | | | | aacatata |
| SEQ_11 | P$OPAQ/O2.01 | Opaque-regulatory protein | 0.87 | 569 | 612 | − | 0.852 | 0.889 | atgttcacaTCAA |
| | | | | | | | | | ctta |
| SEQ_11 | P$NCS1/NCS1.01 | Nodulin consensus sequence 1 | 0.85 | 658 | 668 | − | 0.804 | 0.882 | aAAACgattgt |
| SEQ_11 | P$PREM/ | Promoter elements involved in MgProto | 0.77 | 697 | 727 | − | 1.000 | 0.806 | caatccgtggcga |
| | MGPROTORE.01 | (Mg-protoporphyrin IX) and light- | | | | | | | tttgcact |
| | | mediated induction | | | | | | | |
| SEQ_11 | P$HOCT/HOCT.01 | Octamer motif found in plant histone H4 | 0.76 | 706 | 722 | − | 1.000 | 0.799 | gacggcaATCCgt |
| | | genes | | | | | | | ggcg |
| SEQ_11 | P$DOFF/DOF3.01 | Dof-single zinc finger transcription | 0.99 | 726 | 742 | − | 1.000 | 1.000 | tcgccggaAAAGc |
| | | factor | | | | | | | gcga |
| SEQ_11 | P$CGCG/ATSR1.01 | Arabidopsis thaliana signal-responsive | 0.84 | 748 | 764 | − | 1.000 | 0.904 | aaCGCGttcgcgg |
| | | gene1, Ca2+/ calmodulin binding | | | | | | | tcg |
| | | protein homolog to NtER1 (tobacco | | | | | | | |
| | | early ethylene-responsive gene) | | | | | | | |
| SEQ_11 | P$CGCG/ATSR1.01 | Arabidopsis thaliana signal-responsive | 0.84 | 755 | 771 | + | 1.000 | 0.896 | gaaCGCGttttcg |
| | | gene1, Ca2⇄/ calmodulin binding | | | | | | | aaaa |
| | | protein homolog to NtER1 (tobacco | | | | | | | |
| | | early ethylene-responsive gene) | | | | | | | |
| SEQ_11 | P$SEF3/SEF3.01 | SEF3, Soybean embryo factor 3 | 0.87 | 767 | 781 | − | 1.000 | 0.905 | tttaaACCCattt |
| | | | | | | | | | tc |
| SEQ_11 | P$MYBS/OSMYBS.01 | Rice MYB proteins with single DNA binding | 0.82 | 794 | 810 | − | 1.000 | 0.861 | agaagTATCcaga |
| | | domains, binding to the amylase | | | | | | | caac |
| | | element (TATCCA) | | | | | | | |
| SEQ_11 | P$L1BX/ATML1.01 | L1-specific homeodomain protein ATML1 | 0.82 | 811 | 827 | + | 1.000 | 0.827 | atatttTAAAagt |
| | | (A. thaliana meristem layer 1) | | | | | | | attt |
| SEQ_11 | P$SPF1/SP8BF.01 | DNA-binding protein of sweet potato | 0.87 | 814 | 826 | − | 1.000 | 0.928 | aaTACTtttaaaa |
| | | that binds to the SP8a (ACTGTGTA) and | SP8b | | | | | | |
| | | (TACTATT) sequences of sporamin and | | | | | | | |
| | | beta-amylase genes | | | | | | | |
| SEQ_11 | P$DOFF/PBOX.01 | Prolamin box, conserved in cereal seed | 0.75 | 877 | 893 | + | 1.000 | 0.771 | taactggtAAAGa |
| | | storage protein gene promoters | | | | | | | atat |
| SEQ_11 | P$AHBP/WUS.01 | Homeodomain protein WUSCHEL | 0.94 | 891 | 901 | + | 1.000 | 1.000 | tatttTAATga |
| SEQ_11 | P$GTBX/GT1.01 | GT1-Box binding factors with a | 0.85 | 920 | 936 | + | 0.843 | 0.881 | tctgtgGTGAatg |
| | | trihelix DNA-binding domain | | | | | | | atta |
| SEQ_11 | P$AHBP/ATHB5.01 | HDZip class I protein ATHB5 | 0.89 | 927 | 937 | − | 0.936 | 0.939 | ttaATCAttca |
| SEQ_11 | P$AHBP/HAHB4.01 | Sunflower homeodomain leucine-zipper | 0.87 | 927 | 937 | + | 1.000 | 0.945 | tgaatgATTAa |
| | | protein Hahb-4 | | | | | | | |
| SEQ_11 | P$GTBX/SBF1.01 | SBF-1 | 0.87 | 927 | 943 | + | 1.000 | 0.913 | tgaatgaTTAAat |
| | | | | | | | | | tcac |
| SEQ_11 | P$L1BX/ATML1.01 | L1-specific homeodomain protein ATML | 0.82 | 929 | 945 | + | 1.000 | 0.840 | aatgatTAAAttc |
| | | (A. thaliana meristem layer 1) | | | | | | | acca |
| SEQ_11 | P$AHBP/WUS.01 | Homeodomain protein WUSCHEL | 0.94 | 931 | 941 | − | 1.000 | 0.963 | gaattTAATca |
| SEQ_11 | P$GTBX/GT1.01 | 1-Box binding factors with a trihelix | 0.85 | 933 | 949 | − | 0.843 | 0.919 | agtgtgGTGAatt |
| | | DNA-binding domain | | | | | | | taat |
| SEQ_11 | P$DOFF/PBF.01 | PBF (MPBF) | 0.97 | 943 | 959 | − | 1.000 | 0.984 | ggttatgaAAAGt |
| | | | | | | | | | gtgg |
| SEQ_11 | P$MYBL/GAMYB.01 | GA-regulated myb gene from barley | 0.91 | 950 | 966 | − | 1.000 | 0.919 | tttgattgGTTAt |
| | | | | | | | | | gaaa |
| SEQ_11 | P$CAAT/CAAT.01 | CCAAT-box in plant promoters | 0.97 | 956 | 964 | + | 1.000 | 0.984 | aaCCAAtca |
| SEQ_11 | P$GTBX/SBF1.01 | SBF-1 | 0.87 | 977 | 993 | + | 1.000 | 0.949 | ggagtagTTAAaa |
| | | | | | | | | | aaga |
| SEQ_11 | P$NCS2/NCS2.01 | Nodulin consensus sequence 2 | 0.79 | 984 | 998 | − | 0.750 | 0.846 | tattgtCTTTttt |
| | | | | | | | | | aa |
| SEQ_11 | P$HMGF/HMG_IY.02 | High mobility group I/Y-like protein | 1.00 | 994 | 1008 | − | 1.000 | 1.000 | atttTATTtttat |
| | | isolated from pea | | | | | | | tg |
| SEQ_11 | P$HMGF/HMG_IY.01 | High mobility group I/Y-like proteins | 0.89 | 999 | 1013 | − | 1.000 | 0.924 | ttttTATTttatt |
| | | | | | | | | | tt |
| SEQ_11 | P$MADS/SQUA.01 | MADS-box protein SQUAMOSA | 0.90 | 1001 | 1021 | − | 1.000 | 0.950 | ttcagctATTTtt |
| | | | | | | | | | attttatt |
| SEQ_11 | P$SEF4/SEF4.01 | Soybean embryo factor 4 | 0.98 | 1005 | 1015 | − | 1.000 | 0.985 | taTTTTtattt |
| SEQ_11 | P$CE1F/AB14.01 | ABA insensitive protein 4 (ABI4) | 0.87 | 1026 | 1038 | + | 1.000 | 0.891 | acaaCACCgacat |
| SEQ_11 | P$DREB/ | C-repeat/dehydration response element | 0.89 | 1027 | 1041 | + | 1.000 | 0.962 | caacaCCGAcatt |
| | CRT_DRE.01 | | | | | | | | ga |
| SEQ_11 | P$AHBP/WUS.01 | Homeodomain protein WUSCHEL | 0.94 | 1039 | 1049 | − | 1.000 | 0.963 | gacgtTAATca |
| SEQ_10 | P$TELO/ATPURA.01 | Arabidopsis Telo-box interacting | 0.85 | 1053 | 1067 | + | 0.750 | 0.863 | taaaAACCtagac |
| | | protein related to the conserved | | | | | | | ta |
| | | animal protein Pur-alpha | | | | | | | |
| SEQ_11 | P$TBPF/TATA.02 | Plant TATA box | 0.90 | 1062 | 1076 | + | 1.000 | 0.924 | agacTATAaaacc |
| | | | | | | | | | at |
| SEQ_11 | P$TELO/ATPURA.01 | Arabidopsis Telo-box interacting | 0.85 | 1068 | 1082 | + | 0.750 | 0.868 | taaaACCAtaaat |
| | | protein related to the conserved | | | | | | cc | |
| | | animal protein Pur-alpha | | | | | | | |
| SEQ_11 | O$RPOA/POLYA.01 | Mammalian C-type LTR Poly A signal | 0.76 | 1071 | 1091 | + | 1.000 | 0.804 | aaccaTAAAtcct |
| | | | | | | | | | aaatctga |
| SEQ_11 | P$TELO/ATPURA.01 | Arabidopsis Telo-box interacting | 0.85 | 1075 | 1089 | + | 0.750 | 0.860 | ataaATCCtaaat |
| | | protein related to the conserved | | | | | | | ct |
| | | animal protein Pur-alpha | | | | | | | |
| SEQ_11 | P$CCAF/CCA1.01 | Circadian clock associated 1 | 0.85 | 1077 | 1091 | + | 1.000 | 0.868 | aaatcctaAATCt |
| | | | | | | | | | ga |
| SEQ_11 | P$TELO/ATPURA.01 | Arabidopsis Telo-box interacting | 0.85 | 1089 | 1103 | + | 0.750 | 0.858 | tgaaACACtaaac |
| | | protein related to the conserved | | | | | | | ca |
| | | animal protein Pur-alpha | | | | | | | |
| SEQ_11 | O$RPOA/POLYA.01 | Mammalian C-type LTR Poly A signal | 0.76 | 1092 | 1112 | + | 1.000 | 0.807 | aacacTAAAccat |
| | | | | | | | | | aaatctca |
| SEQ_11 | P$TELO/ATPURA.01 | Arabidopsis Telo-box interacting | 0.85 | 1096 | 1110 | + | 0.750 | 0.862 | ctaaACCAtaaat |
| | | protein related to the conserved | | | | | | | ct |
| | | animal protein Pur-alpha | | | | | | | |
| SEQ_11 | P$CCAF/CCA1.01 | Circadian clock associated 1 | 0.85 | 1098 | 1112 | + | 1.000 | 0.905 | aaaccataAATCt |
| | | | | | | | | | ca |
| SEQ_11 | P$TELO/ATPURA.01 | Arabidopsis Telo-box interacting | 0.85 | 1116 | 1130 | + | 0.750 | 0.857 | ctaaACTCtaaac |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1123 | 1137 | + | 1.000 | 0.985 | ctaaACCCtaaat |
| | | protein related to the conserved | | | | | | | ct |
| | | animal protein Pur-alpha | | | | | | | |
| SEQ_11 | P$CCAF/CCA1.01 | Circadian clock associated 1 | 0.85 | 1125 | 1139 | + | 1.000 | 0.892 | aaaccctaAATCt |
| | | | | | | | | | ta |
| SEQ_11 | P$DOFF/PBOX.01 | Prolamin box, conserved in cereal seed | 0.75 | 1137 | 1153 | − | 1.000 | 0.782 | ttgggtttAAAGt |
| | | storage protein gene promoters | | | | | | | ttaa |
| SEQ_11 | P$GTBX/SBF1.01 | SBF-1 | 0.87 | 1138 | 1154 | − | 1.000 | 0.880 | tttgggtTTAAag |
| | | | | | | | | | ttta |
| SEQ_11 | P$SEF3/SEF3.01 | SEF3, Soybean embryo factor 3 | 0.87 | 1143 | 1157 | + | 1.000 | 0.886 | tttaaACCCaaac |
| | | | | | | | | | tc |
| SEQ_11 | P$TELO/ATPURA.01 | Arabidopsis Telo-box interacting | 0.85 | 1144 | 1158 | + | 1.000 | 0.862 | ttaaACCCaaact |
| | | protein related to the conserved | | | | | | | ct |
| | | animal protein Pur-alpha | | | | | | | |
| SEQ_11 | P$TELO/ATPURA.01 | Arabidopsis Telo-box interacting | 0.85 | 1150 | 1164 | + | 0.750 | 0.859 | ccaaACTCtaaac |
| | | protein related to the conserved | | | | | | | aa |
| | | animal protein Pur-alpha | | | | | | | |
| SEQ_11 | P$STKM/STK.01 | Storekeeper (STK), plant specific DNA | 0.85 | 1155 | 1169 | + | 1.000 | 0.862 | ctcTAAAcaataa |
| | | binding protein important for tuber- | | | | | | | ac |
| | | specific and sucrose-inducible gene | | | | | | | |
| | | expression | | | | | | | |
| SEQ_11 | O$RPOA/LPOLYA.01 | Lentiviral Poly A signal | 0.94 | 1160 | 1180 | + | 1.000 | 0.941 | aacAATAaacctt |
| | | | | | | | | | aaatccta |
| SEQ_11 | P$TELO/ATPURA.01 | Arabidopsis Telo-box interacting | 0.85 | 1164 | 1178 | + | 0.750 | 0.860 | ataaACCTtaaat |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1171 | 1185 | + | 0.750 | 0.850 | ttaaATCCtaaaa |
| | | protein related to the conserved | | | | | | | tc |
| | | animal protein Pur-alpha | | | | | | | |
| SEQ_11 | P$CCAF/CCA1.01 | Circadian clock associated 1 | 0.85 | 1174 | 1188 | + | 1.000 | 0.938 | aatcctaaAATCt |
| | | | | | | | | | aa |
| SEQ_11 | P$LREM/ATCTA.01 | Motif involved in carotenoid and | 0.85 | 1181 | 1191 | + | 1.000 | 0.970 | aaATCTaaacc |
| | | tocopherol biosynthesis and in the | | | | | | | |
| | | expression of photosynthesis-related | | | | | | | |
| | | genes | | | | | | | |
| SEQ_11 | P$TELO/ATPURA.01 | Arabidopsis Telo-box interacting | 0.85 | 1185 | 1199 | + | 1.000 | 0.980 | ctaaACCCtaaac |
| | | protein related to the conserved | | | | | | | cc |
| | | animal protein Pur-alpha | | | | | | | |
| SEQ_11 | P$TELO/RPBX.01 | Ribosomal protein box, appears unique | 0.84 | 1192 | 1206 | + | 0.755 | 0.847 | ctaaaCCCAaagc |
| | | to plant RP genes and genes | | | | | | | ta |
| | | associated with gene expression | | | | | | | |
| SEQ_11 | P$LEGB/LEGB.01 | Legumin box, highly conserved sequence | 0.59 | 1197 | 1223 | + | 0.750 | 0.607 | cccaaagCTATaa |
| | | element about 100 bp upstream of the | | | | | | | accataaaccata |
| | | TSS in legumin genes | | | | | | | a |
| SEQ_11 | P$TELO/ATPURA.01 | Arabidopsis Telo-box interacting | 0.85 | 1206 | 1220 | + | 0.750 | 0.855 | ataaACCAtaaac |
| | | protein related to the conserved | | | | | | | ca |
| | | animal protein Pur-alpha | | | | | | | |
| SEQ_11 | P$TELO/ATPURA.01 | Arabidopsis Telo-box interacting | 0.85 | 1213 | 1227 | + | 0.750 | 0.852 | ataaACCAtaaaa |
| | | protein related to the conserved | | | | | | | tc |
| | | animal protein Pur-alpha | | | | | | | |
| SEQ_11 | P$CCAF/CCA1.01 | Circadian clock associated 1 | 0.85 | 1216 | 1230 | + | 1.000 | 0.950 | aaccataaAATCt |
| | | | | | | | | | aa |
| SEQ_11 | O$RPOA/ | PolyA signal of D-type LTRs | 0.78 | 1217 | 1237 | + | 1.000 | 0.852 | aCCATaaaatcta |
| | DTYPEPA.01 | | | | | | | | aaccctaa |
| SEQ_11 | O$LTUP/TAACC.01 | Lentiviral A upstream element | 0.71 | 1218 | 1240 | + | 1.000 | 0.713 | ccataaaatctaA |
| | | | | | | | | | ACCctaaatc |
| SEQ_11 | P$LREM/ATCTA.01 | Motif involved in carotenoid and | 0.85 | 1223 | 1233 | + | 1.000 | 0.970 | aaATCTaaacc |
| | | tocopherol biosynthesis and in the | | | | | | | |
| | | expression of photosynthesis-related | | | | | | | |
| | | genes | | | | | | | |
| SEQ_11 | P$TELO/ATPURA.01 | Arabidopsis Telo-box interacting | 0.85 | 1227 | 1241 | + | 1.000 | 0.985 | ctaaACCCtaaat |
| | | animal protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1234 | 1248 | + | 0.750 | 0.862 | ctaaATCCtaaat |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1241 | 1255 | + | 0.750 | 0.857 | ctaaATCCtaaac |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1248 | 1262 | + | 1.000 | 0.980 | ctaaACCCtaaac |
| | | protein related to the conserved | | | | | | | tt |
| | | animal protein Pur-alpha | | | | | | | |
| SEQ_11 | P$NCS1/NCS1.01 | Nodulin consensus sequence 1 | 0.85 | 1255 | 1265 | − | 1.000 | 0.895 | aAAAAgtttag |
| SEQ_11 | P$GTBX/SBF1.01 | SBF-1 | 0.87 | 1262 | 1278 | − | 1.000 | 0.889 | tttagggTTAAaa |
| | | | | | | | | | aaaa |
| SEQ_11 | P$TELO/RPBX.01 | Ribosomal protein box, appears unique | 0.84 | 1267 | 1281 | + | 1.000 | 0.898 | tttaaCCCTaaac |
| | | to plant RP genes and genes | | | | | | | tc |
| | | associated with gene expression | | | | | | | |
| SEQ_11 | P$TELO/ATPURA.01 | Arabidopsis Telo-box interacting | 0.85 | 1293 | 1307 | + | 0.750 | 0.855 | tcaaATCCtaaac |
| | | protein related to the conserved | | | | | | tc | |
| | | animal protein Pur-alpha | | | | | | | |
| SEQ_11 | P$TELO/ATPURA.01 | Arabidopsis Telo-box interacting | 0.85 | 1300 | 1314 | + | 0.750 | 0.857 | ctaaACTCtaaac |
| | | protein related to the conserved | | | | | | cc | |
| | | animal protein Pur-alpha | | | | | | | |
| SEQ_11 | P$SEF3/SEF3.01 | SEF3, Soybean embryo factor 3 | 0.87 | 1306 | 1320 | + | 1.000 | 0.891 | tctaaACCCaaaa |
| | | | | | | | | | ct |
| SEQ_11 | P$TELO/RPBX.01 | Ribosomal protein box, appears unique | 0.84 | 1307 | 1321 | + | 0.755 | 0.858 | ctaaaCCCAaaac |
| | | to plant RP genes and genes | | | | | | | tt |
| | | associated with gene expression | | | | | | | |
| SEQ_11 | P$TELO/ATPURA.01 | Arabidopsis Telo-box interacting | 0.85 | 1321 | 1335 | + | 0.750 | 0.855 | tcaaATCCtaaac |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1328 | 1342 | + | 1.000 | 0.861 | ctaaACCCaaac |
| | | protein related to the conserved | | | | | | | ctc |
| | | animal protein Pur-alpha | | | | | | | |
| SEQ_11 | P$TELO/ATPURA.01 | Arabidopsis Telo-box interacting | 0.85 | 1342 | 1356 | + | 1.000 | 0.980 | ctaaACCCtaaac |
| | | protein related to the conserved | | | | | | ct | |
| | | animal protein Pur-alpha | | | | | | | |
| SEQ_11 | P$MYBL/MYBPH3.02 | Myb-like protein of Petunia hybrida | 0.857 | 1357 | 1373 | + | 1.000 | 0.808 | atctgcTAGTtaa |
| | | | | | | | | | taag |
| SEQ_11 | P$OCSE/OCSL.01 | OCS-like elements | 0.69 | 1370 | 1390 | + | 0.769 | 0.706 | taagattaaggtt |
| | | | | | | | | | tACGGttt |
| SEQ_11 | P$AHBP/WUS.01 | Homeodomain protein WUSCHEL | 0.94 | 1372 | 1382 | − | 1.000 | 0.963 | aacctTAATct |
| SEQ_11 | P$TELO/ATPURA.01 | Arabidopsis Telo-box interacting | 0.85 | 1378 | 1392 | − | 0.750 | 0.857 | ctaaACCGtaaac |
| | | protein related to the conserved | | | | | | | ct |
| | | animal protein Pur-alpha | | | | | | | |
| SEQ_11 | P$TELO/ATPURA.01 | Arabidopsis Telo-box interacting | 0.85 | 1392 | 1406 | − | 1.000 | 0.859 | ataaACCCaaacc |
| | | protein related to the conserved | | | | | | | tc |
| | | animal protein Pur-alpha | | | | | | | |
| SEQ_11 | P$TELO/ATPURA.01 | Arabidopsis Telo-box interacting | 0.85 | 1399 | 1413 | − | 0.750 | 0.855 | ataaACCAtaaac |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1413 | 1427 | − | 1.000 | 0.988 | ccaaACCCtaaat |
| | | protein related to the conserved | | | | | | | ca |
| | | animal protein Pur-alpha | | | | | | | |
| SEQ_11 | P$TELO/ATPURA.01 | Arabidopsis Telo-box interacting | 0.85 | 1419 | 1433 | − | 1.000 | 0.857 | ttaaACCCaaacc |
| | | protein related to the conserved | | | | | | | ct |
| | | animal protein Pur-alpha | | | | | | | |
| SEQ_11 | P$CCAF/CCA1 | Circadian clock associated 1 | 0.85 | 1431 | 1445 | − | 1.000 | 0.872 | aaactttaAATCt |
| | | | | | | | | | ta |
| SEQ_11 | P$TELO/RPBX.01 | Ribosomal protein box, appears unique | 0.84 | 1440 | 1454 | − | 0.755 | 0.858 | ctaaaCCCAaaac |
| | | to plant RP genes and genes | | | | | | | tt |
| | | associated with gene expression | | | | | | | |
| SEQ_11 | P$SEF3/SEF3.01 | _SEF3, Soybean embryo factor 3 | 0.87 | 1441 | 1455 | − | 1.000 | 0.872 | cctaaACCCaaaa |
| | | | | | | | | | ct |
| SEQ_11 | P$TELO/ATPURA.01 | Arabidopsis Telo-box interacting | 0.85 | 1447 | 1461 | − | 1.000 | 0.980 | ctaaACCCtaaac |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1454 | 1468 | − | 0.750 | 0.857 | ctaaATCCtaaac |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1461 | 1475 | − | 0.750 | 0.862 | ctaaACTCtaaat |
| | | protein related to the conserved | | | | | | | cc |
| | | animal protein Pur-alpha | | | | | | | |
| SEQ_11 | P$LREM/ATCTA.01 | Motif involved in carotenoid and | 0.85 | 1469 | 1479 | − | 1.000 | 0.970 | aaATCTaaact |
| | | tocopherol biosynthesis and in the | | | | | | | |
| | | expression of photosynthesis-related | | | | | | | |
| | | genes | | | | | | | |
| SEQ_11 | P$CCAF/CCA1.01 | Circadian clock associated 1 | 0.85 | 1472 | 1486 | − | 1.000 | 0.938 | aatcctaaAATCt |
| | | | | | | | | | aa |
| SEQ_11 | P$TELO/ATPURA.01 | Arabidopsis Telo-box interacting | 0.85 | 1475 | 1489 | − | 0.750 | 0.854 | ctaaATCctaaaa |
| | | protein related to the conserved | | | | | | | tc |
| | | animal protein Pur-alpha | | | | | | | |
| SEQ_11 | O$RPOA/POLYA.01 | Mammalian C-type LTR Poly A signal | 0.76 | 1480 | 1500 | − | 0.750 | 0.762 | cacaaTAAGccct |
| | | | | | | | | | aaatccta |
| SEQ_11 | P$TELO/RPBX.01 | Ribosomal protein box, appears unique | 0.84 | 1482 | 1496 | − | 1.000 | 0.926 | ataagCCCTaaat |
| | | to plant RP genes and genes | | | | | | | cc |
| | | associated with gene expression | | | | | | | |
| SEQ_11 | P$TELO/ATPURA.01 | Arabidopsis Telo-box interacting | 0.85 | 1502 | 1516 | − | 1.000 | 0.866 | ctaaACCCaaact |
| | | protein related to the conserved | | | | | | | ct |
| | | animal protein Pur-alpha | | | | | | | |
| SEQ_11 | P$SEF3/SEF3.01 | SEF3, Soybean embryo factor 3 | 0.87 | 1503 | 1517 | − | 1.000 | 0.890 | cctaaACCCaaac |
| | | | | | | | | | tc |
| SEQ_11 | P$TELO/RPBX.01 | Ribosomal protein box, appears unique | 0.84 | 1509 | 1523 | − | 1.000 | 0.977 | ttaaaCCCTaaac |
| | | to plant RP genes and genes | | | | | | | cc |
| | | associated with gene expression | | | | | | | |
| SEQ_11 | P$CCAF/CCA1.01 | Circadian clock associated 1 | 0.85 | 1521 | 1535 | − | 1.000 | 0.901 | aaaccataAATCt |
| | | | | | | | | | ta |
| SEQ_11 | P$TELO/ATPURA.01 | Arabidopsis Telo-box interacting | 0.85 | 1523 | 1537 | − | 0.750 | 0.862 | ctaaACCAtaaat |
| | | protein related to the conserved | | | | | | | ct |
| | | animal protein Pur-alpha | | | | | | | |
| SEQ_11 | P$LREM/ATCT | Motif involved in carotenoid and | 0.85 | 1531 | 1541 | − | 1.000 | 0.970 | aaATCTaaacc |
| | | tocopherol biosynthesis and in the | | | | | | | |
| | | expression of photosynthesis-related | | | | | | | |
| | | genes | | | | | | | |
| SEQ_11 | P$CCAF/CCA1.01 | Circadian clock associated 1 | 0.85 | 1534 | 1548 | − | 1.000 | 0.938 | aatcctaaAATCt |
| | | | | | | | | | aa |
| SEQ_11 | O$RPOA/POLYA.01 | Mammalian C-type LTR Poly A signal | 0.76 | 1541 | 1561 | − | 1.000 | 0.784 | aaccaTAAAtccc |
| | | | | | | | | | aatcctaa |
| SEQ_11 | O$RPOA/POLYA.01 | Mammalian C-type LTR Poly A signal | 0.76 | 1548 | 1568 | − | 1.000 | 0.768 | aacacTAAAccat |
| | | | | | | | | | aaatccca |
| SEQ_11 | P$TELO/ATPURA.01 | Arabidopsis Telo-box interacting | 0.85 | 1550 | 1564 | − | 0.750 | 0.862 | ctaaACCAtaaat |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1557 | 1571 | − | 0.750 | 0.867 | caaaACACtaaac |
| | | protein related to the conserved | | | | | | | ca |
| | | animal protein Pur-alpha | | | | | | | |
| SEQ_11 | P$TELO/RPBX.01 | Ribosomal protein box, appears unique | 0.84 | 1571 | 1585 | − | 1.000 | 0.989 | ataaaCCCTaaat |
| | | to plant RP genes and genes | | | | | | | cc |
| | | associated with gene expression | | | | | | | |
| SEQ_11 | P$TELO/ATPURA.01 | Arabidopsis Telo-box interacting | 0.85 | 1578 | 1592 | − | 0.750 | 0.863 | taaaACCAtaaac |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1586 | 1600 | − | 0.750 | 0.854 | ctaaACTCtaaaa |
| | | protein related to the conserved | | | | | | | cc |
| | | animal protein Pur-alpha | | | | | | | |
| SEQ_11 | P$TELO/ATPURA.01 | Arabidopsis Telo-box interacting | 0.85 | 1593 | 1607 | − | 0.750 | 0.863 | taaaAACCtaaac |
| | | protein related to the conserved | | | | | | | tc |
| | | animal protein Pur-alpha | | | | | | | |
| SEQ_11 | P$HOCT/HOC | Octamer motif found in plant histone | 0.857 | 1618 | 1634 | − | 1.000 | 0.838 | tagcgcgATCCgc |
| | | H3 and H4 genes | | | | | | | aaaa |
| SEQ_11 | P$AHBP/WUS.01 | Homeodomain protein WUSCHEL | 0.94 | 1632 | 1642 | + | 1.000 | 1.000 | ctaatTAATgt |
| SEQ_11 | P$DREB/ | C-repeat/dehydration response element | 0.89 | 1636 | 1650 | − | 1.000 | 0.965 | caacaCCGAcatt |
| | CRT_DRE.01 | | | | | | | | aa |
| SEQ_11 | P$CE1F/ABI4.01 | ABA insensitive protein (ABI4) | 0.87 | 1639 | 1651 | − | 1.000 | 0.891 | acaaCACCgacat |
| SEQ_11 | P$HMGF/HMG_IY.02 | High mobility group I/Y-like protein | 1.00 | 1649 | 1663 | + | 1.000 | 1.000 | tgttTATTttttt |
| | | isolated from pea | | | | | | | ag |
| SEQ_11 | P$STKM/STK.01 | Storekeeper (STK), plant specific DNA | 0.85 | 1651 | 1665 | − | 1.000 | 0.877 | agcTAAAaaaata |
| | | binding protein important for tuber- | | | | | | | aa |
| | | specific and sucrose-inducible gene | | | | | | | |
| | | expression | | | | | | | |
| SEQ_11 | P$OCSE/OCSL.01 | OCS-like elements | 0.69 | 1664 | 1684 | + | 0.769 | 0.726 | ctatttaattttt |
| | | | | | | | | | tACTTtta |
| SEQ_11 | P$STKM/STK.01 | Storekeeper (STK), plant specific DNA | 0.85 | 1667 | 1681 | − | 1.000 | 0.886 | aagTAAAaaatta |
| | | binding protein important for tuber- | | | | | | | aa |
| | | specific and sucrose-inducible gene | | | | | | | |
| | | expression | | | | | | | |
| SEQ_11 | P$L1BX/ATML1.01 | L1-specific homeodomain protein ATML1 | 0.82 | 1674 | 1690 | − | 1.000 | 0.846 | aaacaaTAAAagt |
| | | (A. thaliana meristem layer 1) | | | | | | | aaaa |
| SEQ_11 | P$SPF1/SP8BF.01 | DNA-binding protein of sweet potato that | 0.87 | 1675 | 1687 | + | 1.000 | 0.877 | ttTACTtttattg |
| | | binds to the SP8a (ACTGTGTA) and SP8b | | | | | | | |
| | | (TACTATT) sequences of sporamin and | | | | | | | |
| | | beta-amylase genes | | | | | | | |
| SEQ_11 | P$STKM/STK.01 | Storekeeper (STK), plant specific DNA | 0.85 | 1691 | 1705 | + | 1.000 | 0.872 | tttTAAActattt |
| | | binding protein important for tuber- | | | | | | | at |
| | | specific and sucrose-inducible gene | | | | | | | |
| | | expression | | | | | | | |
| SEQ_11 | P$MADS/SQUA.01 | MADS-box protein SQUAMOSA | 0.90 | 1693 | 1713 | + | 1.000 | 0.942 | ttaaactATTTat |
| | | | | | | | | | atatgaca |
| SEQ_11 | P$TBPF/TATA.02 | Plant A box | 0.90 | 1696 | 1710 | − | 1.000 | 0.956 | cataTATAaatag |
| | | | | | | | | | tt |
| SEQ_11 | P$TBPF/TATA.02 | Plant A box | 0.90 | 1698 | 1712 | − | 1.000 | 0.913 | gtcaTATAtaaat |
| | | | | | | | | | ag |
| SEQ_11 | P$LEGB/LEGB.01 | Legumin box, highly conserved sequence | 0.59 | 1704 | 1730 | − | 0.750 | 0.607 | ttcataaCCAAcc |
| | | element about 100 bp upstream of the TSS | | | | | | | aaaatgtcatata |
| | | in legumin genes | | | | | | | t |
| SEQ_11 | P$MIIG/P_ACT | Maize activator P of flavonoid bio- | 0.93 | 1714 | 1728 | + | 0.966 | 0.973 | ttttGGTTggtta |
| | | synthetic genes | | | | | | | g |
| SEQ_11 | P$MYBL/GAMYB.01 | GA-regulated myb gene from barley | 0.91 | 1715 | 1731 | + | 1.000 | 0.943 | tttggttgGTTAt |
| | | | | | | | | | gaaa |
| SEQ_11 | P$GAPB/GAP.01 | Cis-element in the GAPDH promoters | 0.88 | 1722 | 1736 | + | 1.000 | 0.933 | ggttATGAaaagt |
| | | conferring light inducibility | | | | | | | ac |
| SEQ_11 | P$GTBX/GT1.01 | GT1-Box binding factors with a tri- | 0.85 | 1732 | 1748 | + | 0.843 | 0.889 | agtacgGTGAatt |
| | | helix DNA-binding domain | | | | | | | taac |
| SEQ_11 | P$L1BX/ATML1.01 | L1-specific homeodomain protein ATML1 | 0.82 | 1736 | 1752 | − | 1.000 | 0.823 | gaatgtTAAAttc |
| | | (A. thaliana meristem layer 1) | | | | | | | accg |
| SEQ_11 | P$GTBX/SBF1.01 | SPF-1 | 0.87 | 1738 | 1754 | − | 1.000 | 0.894 | gtgaatgTTAAat |
| | | | | | | | | | tcac |
| SEQ_11 | P$GTBX/GT1.01 | GT1-Box binding factors with a tri- | 0.85 | 1744 | 1760 | − | 0.843 | 0.889 | cctacgGTGAatg |
| | | helix DNA-binding domain | | | | | | | ttaa |
| SEQ_11 | O$RPOA/ | PolyA signal of D-type LTRs | 0.78 | 1771 | 1791 | − | 0.750 | 0.834 | aCAATtaaaatat |
| | aacaatac | | | | | | | | aacaatac |
| SEQ_11 | O$RPOA/APOLYA.01 | Avian C-type LTR PolyA signal | 0.71 | 1798 | 1818 | − | 0.750 | 0.717 | aacaaTCAAacat |
| | | | | | | | | | cacttgga |
| SEQ_11 | P$CARM/CARICH.01 | CA-rich element | 0.78 | 1807 | 1825 | − | 1.000 | 0.785 | cttgtcaAACAat |
| | | | | | | | | | caaaca |
| SEQ_11 | P$WBXF/WRKY.01 | WRKY plant specific zinc-finger-type | 0.92 | 1813 | 1829 | + | 1.000 | 0.964 | attgtTTGAcaag |
| | | factor associated with pathogen defence, | | | | | | | gtca |
| | | W box | | | | | | | |
| SEQ_10 | P$LEGB/RY.01 | RY and Sph motifs conserved in seed- | 0.87 | 1825 | 1851 | − | 1.000 | 0.939 | ggttgtaaCATGc |
| | | specific promoters | | | | | | | atgtttccgtgac |
| | | | | | | | | | c |
| SEQ_11 | P$IDRE/IDE1.01 | Iron-deficiency-responsive element | 0.77 | 1828 | 1842 | − | 1.000 | 0.786 | atGCATgtttccg |
| | | | | | | | | | tg |
| SEQ_10 | P$LEGB/RY.01 | Y and Sph motifs conserved in seed- | 0.87 | 1828 | 1854 | + | 1.000 | 0.939 | cacggaaaCATGc |
| | | specific promoters | | | | | | | atgttacaaccga |
| | | | | | | | | | t |
| SEQ_10 | P$OPAQ/ | Recognition site for BZIP trans- | 0.81 | 1834 | 1850 | − | 1.000 | 0.838 | gttgtaACATgca |
| | O2_GCN4.01 | cription factors that belong to the | | | | | | | tgtt |
| | | group of Opaque-2 like proteins | | | | | | | |
| SEQ_11 | P$GTBX/GT3A.01 | Trihelix DNA-binding factor GT-3a | 0.83 | 1837 | 1853 | + | 1.000 | 0.843 | atgcatGTTAcaa |
| | | | | | | | | | ccga |
| SEQ_11 | P$GTBX/GT3A.01 | Trihelix DNA-binding factor GT-3a | 0.83 | 1846 | 1862 | + | 0.750 | 0.852 | acaaccGATAcaa |
| | | | | | | | | | tgat |
| SEQ_11 | P$GBOX/GBF1.01 | bZIP protein G-Box binding factor 1 | 0.94 | 1914 | 1934 | − | 1.000 | 0.961 | tgcttgctACGTg |
| | | | | | | | | | tcaacacc |
| SEQ_11 | P$GBOX/HBP1A.01 | HBP-1a, suggested to be involved in | 0.88 | 1915 | 1935 | + | 1.000 | 0.899 | gtgttgaCACGta |
| | | the cell cycle-dependent expression | | | | | | | gcaagcat |
| SEQ_11 | P$ABRE/ABRE.01 | ABA response elements | 0.82 | 1916 | 1932 | + | 1.000 | 0.980 | tgttgacACGTag |
| | | | | | | | | | caag |
| SEQ_11 | P$ABRE/ABRE.01 | ABA response elements | 0.82 | 1917 | 1933 | − | 1.000 | 0.950 | gcttgctACGTgt |
| | | | | | | | | | caac |
| SEQ_11 | P$OPAQ/RITA1.01 | Rice transcription activator-1 | 0.95 | 1917 | 1933 | + | 1.000 | 0.966 | gttgacACGTagc |
| | | (RITA), basic leucin zipper protein, | | | | | | | aagc |
| | | highly expressed during seed | | | | | | | |
| | | development | | | | | | | |
| SEQ_11 | P$IDRE/IDE1.01 | Iron-deficiency-responsive element 1 | 0.77 | 1926 | 1940 | + | 0.777 | 0.831 | taGCAAgcatctt |
| | | | | | | | | | tc |
| SEQ_11 | P$MYBL/GAMYB.01 | GA-regulated myb gene from barley | 0.91 | 1934 | 1950 | + | 1.000 | 0.928 | atctttcaGTTAa |
| | | | | | | | | | ccat |
| SEQ_11 | P$OCSE/OCSL.01 | OCS-like elements | 0.69 | 1938 | 1958 | + | 1.000 | 0.747 | ttcagttaaccat |
| | | | | | | | | | aACGTgtc |
| SEQ_11 | P$MYBS/OSMYBS.01 | Rice MYB proteins with single DNA | 0.82 | 1939 | 1955 | + | 0.750 | 0.829 | tcagtTAACcata |
| | | binding domains, binding to the | | | | | | | acgt |
| | | amylase element (TATCCA) | | | | | | | |
| SEQ_11 | P$GBOX/HBP1B.01 | Wheat bZIP transcription factor HBP1B | 0.83 | 1943 | 1963 | − | 1.000 | 0.840 | gtcgtgacACGTt |
| | | (histone gene binding protein 1b) | | | | | | | atggttaa |
| SEQ_11 | P$GBOX/GBF1.01 | bZIP protein G-Box binding factor 1 | 0.94 | 1944 | 1964 | + | 1.000 | 0.973 | taaccataACGTg |
| | | | | | | | | | tcacgaca |
| SEQ_11 | P$ABRE/ABF1.03 | ABA (abscisic acid) inducible trans- | 0.82 | 1945 | 1961 | + | 1.000 | 0.872 | aaccataaCGTGt |
| | | criptional activator | | | | | | | cacg |
| SEQ_11 | P$GBOX/ | bZIP transcription factor from | 0.77 | 1950 | 1970 | − | 0.750 | 0.779 | ctgtgtTGTCgtg |
| | BZ1P910.01 | Antirrhinum majus | | | | | | | acacgtta |
| SEQ_11 | P$ABRE/ABF1.03 | ABA (abscisic acid) inducible trans- | 0.852 | 1953 | 1969 | − | 1.000 | 0.853 | tgtgttgtCGTGa |
| | | criptional activator | | | | | | | cacg |
| SEQ_11 | P$ERSE/ERSE_I.01 | ERSE I (ER stress-response element | 0.79 | 1953 | 1971 | − | 1.000 | 0.822 | cctgtgttgtcgt |
| | | I)-like motif | | | | | | | gaCACG |
| SEQ_10 | P$IDDF/ID1.01 | Maize INDETERMINE1 zinc finger | 0.92 | 1957 | 1969 | − | 1.000 | 0.927 | tgtgTTGTcgtga |
| | | protein | | | | | | | |
| SEQ_11 | O$LDPS/ | Lentiviral Poly A downstream element | 0.89 | 1958 | 1972 | − | 0.980 | 0.904 | tcCTGTgttgtcg |
| | LDSPOLYA.01 | | | | | | | | tg |
| SEQ_11 | O$RPAD/PADS.01 | Mammalian C-type LTR Poly A down- | 0.87 | 1959 | 1971 | − | 0.906 | 0.892 | cctGTGTtgtcgt |
| | | stream element | | | | | | | |
| SEQ_11 | P$MYBS/MYBST1.01 | MybSt1 (Myb Solanum tuberosum 1) | 0.90 | 1963 | 1979 | − | 1.000 | 0.938 | cgtgttATCCtgt |
| | | with a single myb repeat | | | | | | | gttg |
| SEQ_11 | P$IBOX/GATA.01 | Class I GATA factors | 0.93 | 1966 | 1982 | + | 1.000 | 0.970 | cacagGATAacac |
| | | | | | | | | | gtac |
| SEQ_11 | P$GBOX/GBF1.01 | bZIP protein G-Box binding factor 1 | 0.94 | 1968 | 1988 | − | 1.000 | 0.949 | gatcttgtACGTg |
| | | | | | | | | | ttatcctg |
| SEQ_11 | P$ABRE/ABRE.01 | ABA response elements | 0.82 | 1971 | 1987 | − | 1.000 | 0.865 | atcttgtACGTgt |
| | | | | | | | | | tatc |
| SEQ_11 | P$MADS/AGL15.01 | AGL15, Arabidopsis MADS-domain protein | 0.79 | 1977 | 1997 | + | 0.775 | 0.802 | acgTACAagatcg |
| | | AGAMOUS-like 15 | | | | | | | agaaaccg |
| SEQ_11 | O$LTUP/TAACC.01 | Lentiviral TATA upstream element | 0.71 | 1981 | 2003 | + | 1.000 | 0.710 | acaagatcgagaA |
| | | | | | | | | | ACCgcatata |
| SEQ_11 | P$MYBS/ | MYB protein from wheat | 0.83 | 1990 | 2006 | − | 1.000 | 0.913 | tagtATATgcggt |
| | TAMYB80.01 | | | | | | | | ttct |
| SEQ_11 | P$MYBS/ | MYB protein from wheat | 0.83 | 1995 | 2011 | + | 1.000 | 0.911 | ccgcATATactaa |
| | TAMYB80.01 | | | | | | | | acac |
| SEQ_11 | P$LFYB/LFY.01 | Plant specific floral meristem | 0.93 | 2004 | 2016 | − | 0.885 | 0.950 | tGCCAgtgtttag |
| | | identity gene LEAFY (LFY) | | | | | | | |
| SEQ_11 | P$DOFF/DOF2.01 | Dof2-single zinc finger transcription | 0.98 | 2045 | 2061 | − | 1.000 | 0.981 | ggagattaAAAGc |
| | | factor | | | | | | | taat |
| SEQ_11 | P$GTBX/SBF1.01 | SBF-1 | 0.87 | 2047 | 2063 | − | 1.000 | 0.897 | gtggagaTTAAaa |
| | | | | | | | | | gcta |
| SEQ_11 | P$AHBP/WUS.01 | Homeodomain protein WUSCHEL | 0.94 | 2049 | 2059 | + | 1.000 | 0.963 | gctttTAATct |
| SEQ_11 | P$MIIG/ | Putative cis-acting element in various | 0.81 | 2058 | 2072 | − | 0.936 | 0.865 | gcGTGGtgtgtgg |
| | PALBOXP.01 | PAL and 4CL gene promoters | | | | | | | ag |
| SEQ_11 | O$MINI/ | Muscle Initiator Sequence | 0.86 | 2061 | 2079 | + | 1.000 | 0.879 | cacacaCCACgca |
| | MUSCLE_INI.02 | | | | | | | | gctata |
| SEQ_11 | P$GBOX/GBF1.01 | bZIP protein G-Box binding factor 1 | 0.94 | 2072 | 2092 | − | 1.000 | 0.976 | tagaagacACGTg |
| | | | | | | | | | tatagctg |
| SEQ_11 | P$GBOX/CPRF.01 | Common plant regulatory factor (CPRF) | 0.95 | 2073 | 2093 | + | 1.000 | 0.985 | agctatacACGTg |
| | | from parsley | | | | | | | tcttctat |
| SEQ_11 | P$MYCL/MYCRS.01 | Myc recognition sequences | 0.93 | 2073 | 2091 | − | 1.000 | 0.965 | agaagacACGTgt |
| | | | | | | | | | atagct |
| SEQ_11 | P$ABRE/ABRE.01 | ABA response elements | 0.82 | 2074 | 2090 | + | 1.000 | 0.884 | gctatacACGTgt |
| | | | | | | | | | cttc |
| SEQ_11 | P$CE3S/CE3.01 | Coupling element 3 (CE3), non-ACGT ABRE | 0.77 | 2074 | 2092 | + | 0.750 | 0.783 | gctataCACGtgt |
| | | | | | | | | | cttcta |
| SEQ_11 | P$MYCL/ICE.01 | ICE (inducer of CBF expression 1), | 0.95 | 2074 | 2092 | + | 0.954 | 0.955 | tctatACACgtgt |
| | | AtMYC2 (rd22BP1) | | | | | | | cttcta |
| SEQ_11 | P$ABRE/ABF1.01 | ABA (abscisic acid) inducible trans- | 0.79 | 2075 | 2091 | − | 1.000 | 0.824 | agaagACACgtgt |
| | | criptional activator | | | | | | | atag |
| SEQ_11 | P$DPBF/DPBF.01 | bZIP factors DPBF-1 and 2 (Dc3 | 0.89 | 2078 | 2088 | + | 1.000 | 0.908 | tACACgtgtct |
| | | promoter binding factor-1 and 2) | | | | | | | |
| SEQ_11 | P$CGCG/OSCBT.01 | Oryza sativa CaM-binding transcription | 0.78 | 2079 | 2095 | + | 0.906 | 0.804 | acaCGTGtcttct |
| | | factor | | | | | | | atgc |
| SEQ_11 | P$GBOX/CPRF.01 | Common plant regulatory factor (CPRF) | 0.95 | 2091 | 2111 | − | 1.000 | 0.968 | aacaaggcACGTg |
| | | from parsley | | | | | | | ttggcata |
| SEQ_11 | P$GBOX/CPRF.01 | Common plant regulatory factor (CPRF) | 0.95 | 2092 | 2112 | + | 1.000 | 0.968 | atgccaacACGTg |
| | | from parsley | | | | | | | ccttgttc |
| SEQ_11 | P$MYCL/ | Rice bHLH protein | 0.85 | 2092 | 2110 | − | 1.000 | 0.937 | acaaggCACGtgt |
| | OSBHLH66.01 | | | | | | | | tggcat |
| SEQ_11 | P$ABRE/ABF1.01 | ABA (abscisic acid) inducible trans- | 0.79 | 2093 | 2109 | + | 1.000 | 0.852 | tgccaACACgtgc |
| | | criptional activator | | | | | | | cttg |
| SEQ_11 | P$CE3S/CE3.01 | Coupling element 3 (CE3), non-ACGT | 0.77 | 2093 | 2111 | + | 0.750 | 0.776 | tgccaaCACGtgc |
| | | ABRE | | | | | | | cttgtt |
| SEQ_11 | P$MYCL/ | Rice bHLH protein | 0.85 | 2093 | 2111 | + | 1.000 | 0.950 | tgccaaCACGtgc |
| | OSBHLH66.01 | | | | | | | | cttgtt |
| SEQ_11 | P$MIIG/ | Cis-acting element conserved in | 0.80 | 2113 | 2127 | − | 0.785 | 0.806 | ccttggtcGGTTt |
| | PALBOXL.01 | various PAL and 4CL promoters | | | | | | | ga |
| SEQ_11 | P$TBPF/TATA.02 | Plant TATA box | 0.90 | 2129 | 2143 | + | 1.000 | 0.943 | acacTATAaatgt |
| | | | | | | | | | ct |
| SEQ_11 | P$MYBS/MYBST1.01 | MybSt1 (Myb Solanum tuberosum 1) | 0.90 | 2146 | 2162 | − | 1.000 | 0.943 | attgttATCCaga |
| | | with a single myb repeat | | | | | | | ccat |
| SEQ_11 | P$IBOX/GATA.01 | Class I factors | 0.93 | 2149 | 2165 | + | 1.000 | 0.949 | gttctgGATAaca |
| | | | | | | | | | ataca |
| SEQ_11 | P$MYBL/GAMYB.01 | GA-regulated myb gene from barley | 0.91 | 2151 | 2167 | − | 1.000 | 0.916 | tgtgtattGTTAt |
| | | | | | | | | | ccag |
| SEQ_11 | O$LDPS/ | Lentiviral Poly A downstream element | 0.89 | 2156 | 2170 | − | 0.980 | 0.910 | aaCTGTgtattgt |
| | LDSPOLYA.01 | | | | | | | | ta |
| SEQ_11 | P$MYBL/MYBPH3.02 | Myb-like protein of Petunia hybrida | 0.76 | 2175 | 2191 | − | 0.778 | 0.761 | aaatgtTCGTtgc |
| | | | | | | | | | tgtg |
| SEQ_11 | P$GTBX/GT1.01 | GT1-Box binding factors with a tri- | 0.85 | 2183 | 2199 | − | 0.968 | 0.876 | ttttctGTAAatg |
| | | helix DNA-binding domain | | | | | | | ttcg |
| SEQ_11 | P$DOFF/DOF1.10 | Dof1/MNB1a single zinc finger trans- | 0.98 | 2197 | 2213 | − | 1.000 | 0.984 | tgaaagttAAAGc |
| | | cription factor | | | | | | | tttt |
| SEQ_11 | P$L1BX/ATML1.01 | L1-specific homeodomain protein ATML1 | 0.82 | 2207 | 2223 | − | 0.750 | 0.823 | aaatagGAAAtga |
| | | (A. thaliana meristem layer 1) | | | | | | | aagt |
| SEQ_11 | P$MADS/AG.01 | Agamous, required for normal flower | 0.80 | 2212 | 2232 | + | 0.962 | 0.810 | catTTCCtatttg |
| | | development, similarity to SRF | | | | | | | cgcatttg |
| | | (human) and MCM (yeast) proteins | | | | | | | |

TABLE 6
cis- regulatory elements of SEQ ID NO: 12

| | | | | | | | | |
|---|---|---|---|---|---|---|---|---|
| SEQ_12 | P$DOFF/PB | Prolamin box, conserved in cereal seed | 0.75 | 6 | 22 + 1.000 | 0.759 | tcacaagcA- | |
| | OX.01 | storage protein gene promoters | | | | | AAGagaaa | |
| SEQ_12 | O$LTUP/TA | Lentiviral TATA upstream element | 0.71 | 9 | 31 + 1.00 | 0.737 | caagcaaagaga- | |
| | ACC.01 | | | | | | AACCctaatac | |
| SEQ_12 | P$PSRE/GA | GAAA motif involved in pollen | 0.83 | 14 | 30 + 1.00 | 0.833 | aaagaGA- | |
| | AA.01 | specific transcriptional activation | | | | | | AAccctaata |
| SEQ_12 | P$TELO/RP | Ribosomal protein box, appears unique | 0.84 | 18 | 30 + 1.000 | 0.993 | agaaaCCC- | |
| | BX.01 | to plant RP genes and genes associated | | | | | | Taatacg |
| | | with gene expression | | | | | | |
| SEQ_12 | P$MYBL/MY | Myb-like protein of Petunia hybrida | 0.80 | 37 | 53 + 0.750 | 0.845 | aaaaaacgAT- | |
| | BPH3.01 | | | | | | TAatgat | |
| SEQ_12 | P$NCS1/NC | Nodulin consensus sequence 1 | 0.85 | 39 | 49 + 0.804 | 0.924 | aAAACgattaa | |
| | S1.01 | | | | | | aAAACgattaa | |
| SEQ_12 | P$AHBP1WU | Homeodomain protein WUSCHEL | 0.94 | 42 | 52 +1.000 | 1.000 | acgatTAATga | |
| | S.01 | | | | | | | |
| SEQ_12 | P$AHBP1WU | Homeodomain protein WUSCHEL | 0.94 | 43 | 53 −1.000 | 0.963 | atcat- | |
| | S.01 | | | | | | TAATcg | |
| SEQ_12 | P$AHBP/HA | Sunflower homeodomain leucine-zipper | 0.87 | 46 | 56 − 1.000 | 0.940 | tttatcATTAa | |
| | HB4.01 | protein Hahb-4 | | | | | | |
| SEQ_12 | P$DOFF/DO | Dof2-single zinc finger transcription | 0.98 | 46 | 62 + 1.000 | 0.986 | ttaatgatA- | |
| | F2.01 | factor | | | | | AAGctggt | |
| SEQ_12 | P$1BOX/GAT | Class I GATA factors | 0.93 | 46 | 62 + 1.000 | 0.960 | ttaatGATA- | |
| | A.01 | | | | | | aagctggt | |
| SEQ_12 | P$MADS/AG | AGL3, MADS Box protein | 0.83 | 77 | 97 − 0.973 | 0.900 | tccttCCAAat- | |
| | L3.01 | | | | | | gaagaaggca | |
| SEQ_12 | P$GAPB/GA | Cis-element in the GAPDH promoters | 0.88 | 78 | 92 − 1.000 | 0.926 | ccaaATGAa- | |
| | P.01 | conferring light inducibility | | | | | | gaaggc |
| SEQ_12 | P$MADS/AG | AGL15, Arabidopsis MADS-domain protein | 0.79 | 78 | 98 + 0.925 | 0.807 | gccTTCTtcattt | |
| | L15.01 | AGAMOUS-like 15 | | | | | | ggaaggag |
| SEQ_12 | P$E2FF/E2F. | E2F class I sites | 0.82 | 92 | 106 − 1.000 | 0.832 | tttcTTCCctcctt | |
| | 01 | | | | | | c | |
| SEQ_12 | P$GTBX/GT | Trihelix DNA-binding factor GT-3a | 0.83 | 157 | 173 + 0.750 | 0.839 | atcaacCTTAc- | |
| | 3A.01 | | | | | | cattac | |
| SEQ_12 | P$1BOX/IBO | I-Box in rbcS genes and other light | 0.81 | 157 | 173 − 0.750 | 0.822 | gtaatGG- | |
| | X.01 | regulated genes | | | | | TAaggttgat | |
| SEQ_12 | P$NCS1/NC | Nadulin consensus sequence 1 | 0.85 | 178 | 188 + 1.000 | 0.852 | aAAAAgttaaa | |
| | S1.01 | | | | | | | |
| SEQ_12 | O$RPOA/PP | Mammalian C-type LTR Poly A signal | 0.76 | 188 | 208 + 1.000 | 0.820 | aggagTAA- | |
| | LYA.01 | | | | | | Aaccttaccatta | |
| SEQ_12 | P$GTBX/GT | Trihelix DNA-binding factor GT-3a | 0.83 | 193 | 209 + 0.750 | 0.820 | taaaacCTTAc- | |
| | 3A.01 | | | | | | cattac | |
| SEQ_12 | P$1BOX/IBO | I-Box in rbcS genes and other light | 0.81 | 193 | 209 − 0.750 | 0.829 | gtaatGG- | |
| | X.01 | regulated genes | | | | | TAaggtttta | |
| SEQ_12 | P$CARM/CA | CA-rich element | 0.78 | 222 | 240 + 1.000 | 0.785 | gtcctgaAACA- | |
| | RICH.01 | | | | | | aataaaac | |
| SEQ_12 | P$TBPF/TAT | Plant TATA box | 0.90 | 238 | 252 + 1.000 | 0.915 | aacaTATAta- | |
| | A.02 | | | | | | aactg | |
| SEQ_12 | P$TBPF/TAT | Plant TATA box | 0.88 | 240 | 254 + 1.000 | 0.892 | cataTATA- | |
| | A.01 | | | | | | aactgat | |
| SEQ_12 | P$MYBL/GA | GA-regulated myb gene from barley | 0.91 | 272 | 288 − 1.000 | 0.975 | attggtttGTTA- | |
| | MYB.01 | | | | | | taagg | |
| SEQ_12 | P$CAAT/CA | CCAAT-box in plant promoters | 0.97 | 282 | 290 + 1.000 | 0.975 | | |
| | AT.01 | | | | | | aaCCAAtct | |
| SEQ_12 | P$AHBP/AT | HD-ZIP class III protein ATHB9 | 0.77 | 284 | 294 − 0.750 | 0.780 | | |
| | HB9.01 | | | | | | gtaAAGAttgg | |
| SEQ_12 | P$DOFF/PB | Prolamin box, conserved in cereal seed | 0.75 | 284 | 300 − 1.000 | 0.777 | ataactgtAAA- | |
| | OX.01 | storage protein gene promoters | | | | | | Gattgg |
| SEQ_12 | P$MYBL/AT | R2R3-type myb-like transcription factor | 0.87 | 288 | 304 + 1.000 | 0.889 | tctttaCAGTta- | |
| | MYB77.01 | (I-type binding site) | | | | | | tagtg |
| SEQ_12 | P$NACF/TA | Wheat NACdomain DNA binding factor | 0.68 | 306 | 328 − 0.812 | 0.749 | gaggtataaca- | |
| | NAC69.01 | | | | | | GACGatagtata | |
| SEQ_12 | P$MYBL/GA | GA-regulated myb gene from barley | 0.91 | 311 | 327 + 1.000 | 0.936 | tatcgtctGTTA- | |
| | MYB.01 | | | | | | tacct | |
| SEQ_12 | P$1DDF/ID1. | Maize INDETERMINATE1 zinc finger protein | 0.92 | 326 | 338 + 1.000 | 0.953 | ctctTTGTcggg | |
| | 01 | | | | | | g | |
| SEQ_12 | P$TCPF/PC | TCP class II transcription factor | 0.95 | 332 | 344 − 0.869 | 0.970 | tgtgGACCcc- | |
| | F5.01 | | | | | | gac | |
| SEQ_12 | P$DOFF/PB | PBF (MPBF) | 0.97 | 358 | 374 + 1.000 | 0.984 | aagtctgaAAA- | |
| | F.01 | | | | | | Gagaag | |
| SEQ_12 | P$MADS/SQ | MADS-box protein SQUAMOSA | 0.90 | 377 | 397 − 1.000 | 0.936 | gtcttctATTTt- | |
| | UA.01 | | | | | | tactccttt | |
| SEQ_12 | P$GTBX/SB | SBF-1 | 0.87 | 437 | 453 − 1.000 | 0.940 | catgttgTTAAa- | |
| | F1.01 | | | | | | taccc | |
| SEQ_12 | P$HEAT/HS | Heat shock element | 0.81 | 470 | 484 − 1.000 | 0.829 | tgattcctgaA- | |
| | E.01 | | | | | | GAAg | |
| SEQ_12 | P$HEAT/HS | Heat shock element | 0.81 | 478 | 492 + 0.826 | 0.838 | 47 49 | 0.80.8 ggaatcatatG- |
| | E.01 | | | | | | GAAc | |
| SEQ_12 | P$CCAF/CC | Circadian clock associated 1 | 0.85 | 505 | 519 + 1.000 | 0.863 | cttcaaa- | |
| | A1.01 | | | | | | gAATCtca | |
| SEQ_12 | 0$RPOA/P0 | Mammalian C-type LTR Poly A signal | 0.76 | 517 | 537 − 1.000 | 0.777 | ataaaTAA- | |
| | LYA.01 | | | | | | Aatccatgagtga | |
| SEQ_12 | P$GTBX/S1F | S1F, site 1 binding factor of spinach | 0.79 | 519 | 535 + 1.000 | 0.838 | actcATGGattt- | |
| | .01 | rps1 promoter | | | | | tattt | |
| SEQ_12 | P$TBPF/TAT | Plant TATA box | 0.90 | 528 | 542 − 1.000 | 0.984 | ctgcTATAaa- | |
| | A.02 | | | | | | taaaa | |
| SEQ_12 | P$MYBL/AT | R2R3-type myb-like transcription factor | 0.87 | 564 | 580 + 0.857 | 0.876 | tccggaCCGTtt | |
| | MYB77.01 | (I-type binding site) | | | | cacaa | | |
| SEQ_12 | P$MSAE/MS | M-phase-specific activators | 0.80 | 565 | 579 − 1.000 | 0.873 | tgtga- | |
| | A.01 | (NtmybA1, NtmybA2, NtmybB) | | | | | AACGgtccgg | |
| SEQ_12 | P$GBOX/HB | Wheat bZIP transcription factor HBP1B | 0.83 | 595 | 615 − 1.000 | 0.989 | aattttttACGT- | |
| | P1B.01 | (histone gene binding protein 1b) | | | | | caggtagaa | |
| SEQ_12 | P$GBOX/TG | Arabidopsis leucine zipper protein TGA1 | 0.90 | 596 | 616 + 1.000 | 0.989 | tctaccTGACg- | |
| | A1.01 | | | | | | taaaaaattg | |
| SEQ_12 | P$OCSE/OC | OCS-like elements | 0.69 | 601 | 621 − 1.000 | 0.695 | caaaacaattttt- | |
| | SL.01 | | | | | | tACGTcag | |
| SEQ_12 | O$RVUP/LT | Upstream element of C-type Long Terminal | 0.76 | 603 | 523 − 0.761 | 0.775 | ttcaaaa- | |
| | RUP.01 | Repeats | | | | | caatTTTT_ | |
| | | | | | | | tacgtc | |
| SEQ_12 | P$STKM/ST | Storekeeper (STK), plant specific DNA | 0.85 | 604 | 618 + 1.000 | 0.864 | acgTAAAa- | |
| | K.01 | binding protein important for tuber- | | | | | | aattgtt |
| | | specific and sucrose-inducible gene | | | | | | |
| | | expression | | | | | | |
| SEQ_12 | P$STKM/ST | Storekeeper (STK), plant specific DNA | 0.85 | 609 | 623 − 0.785 | 0.889 | ttcAAAAcaattttt | |
| | K.01 | binding protein important for tuber- | | | | | | |
| | | specific and sucrose-inducible gene | | | | | | |
| | | expression | | | | | | |
| SEQ_12 | P$AHBP/AT | HD-ZIP class III protein ATHB9 | 0.77 | 621 | 631 + 1.000 | 0.775 | gaaATGAtcaa | |
| | HB9.01 | | | | | | | |
| SEQ_12 | P$NCS1/NC | Nodulin consensus sequence 1 | 0.85 | 621 | 631 + 0.878 | 0.933 | gAAATgatcaa | |
| | S1.01 | | | | | | | |
| SEQ_12 | P$URNA/US | Upstream sequence elements in the | 0.75 | 634 | 650 + 0.750 | 0.772 | aaagtaTCACa- | |
| | E.01 | promoters of U-snRNA genes of higher | | | | | tagaaa | |
| | | plants | | | | | | |
| SEQ_12 | P$TBPF/TAT | Plant TATA box | 0.90 | 649 | 663 + 1.000 | 0.904 | aaacTATAca- | |
| | A.02 | | | | | | aataa | |
| SEQ_12 | P$PSRE/GA | GAAA motif involved in pollen specific | 0.83 | 652 | 668 + 0.750 | 0.845 | ctataCAAAtaa- | |
| | AA.01 | transcriptional activation | | | | | tatct | |
| SEQ_12 | P$MADS/SQ | MADS-box protein SQUAMOSA | 0.90 | 662 | 682 + 1.000 | 0.925 | aatatctATTTttt- | |
| | UA.01 | | | | | | tacataa | |
| SEQ_12 | P$LREM/AT | Motif involved in carotenoid and | 0.85 | 663 | 673 + 1.000 | 0.858 | atATCTatttt | |
| | CTA.01 | tocopherol biosynthesis and in the | | | | | | |
| | | expression of photosynthesis-related genes | | | | | | |
| SEQ_12 | P$AHBP/AT | HDZip class I protein ATHB5 | 0.89 | 679 | 689 + 0.936 | 0.939 | ataATCAttgt | |
| | HB5.01 | | | | | | | |
| SEQ_12 | P$AHBP/HA | Sunflower homeodomain leucine-zipper | 0.87 | 679 | 689 − 1.000 | 0.966 | acaatgAT- | |
| | HB4.01 | protein Hahb-4 | | | | | TAt | |
| SEQ_12 | P$NCS2/NC | Nodulin consensus sequence 2 | 0.79 | 687 | 701 − 1.000 | 0.796 | aatagaCTCT- | |
| | S2.01 | | | | | | taaca | |
| SEQ_12 | P$GAPB/GA | Cis-element in the GAPDH promoters | 0.88 | 694 | 708 − 1.000 | 0.902 | aaatATGAata- | |
| | P.01 | conferring light inducibility | | | | | gact | |
| SEQ_12 | P$1DDF/ID1. | Maize INDETERMINATE1 zinc finger protein | 0.92 | 716 | 728 − 1.000 | 0.935 | gaatTTGTcca- | |
| | 01 | | | | | | ca | |
| SEQ_12 | P$AHBP/HA | Sunflower homeodomain leucine-zipper | 0.87 | 728 | 738 + 1.000 | 0.921 | cgtattATTAc | |
| | HB4.01 | protein Hahb-4 | | | | | | |
| SEQ_12 | P$SPF1/SP8 | DNA-binding protein of sweet potato that | 0.87 | 734 | 746 + 1.000 | 0.894 | atTACTtttcttg | |
| | BF.01 | binds to the SP8a (ACTGTGTA) and SP8b | | | | | | |
| | | (TACTATT) sequences of sporamin and beta- | | | | | | |
| | | amylase genes | | | | | | |
| SEQ_12 | P$SEF4/SEF | Soybean embryo factor 4 | 0.98 | 745 | 755 + 1.000 | 0.982 | tgTTTTtgttt | |
| | 4.01 | | | | | | | |
| SEQ_12 | P$NCS1/NC | Nodulin consensus sequence 1 | 0.85 | 765 | 774 − 0.878 | 0.865 | aAAATgatagt | |
| | S1.01 | | | | | | | |
| SEQ_12 | P$DOFF/PB | Prolamin box, conserved in cereal seed | 0.75 | 770 | 786 − 0.776 | 0.771 | tgaaatctAAA- | |
| | OX.01 | storage protein gene promoters | | | | | Taaaat | |
| SEQ_12 | P$LREM/AT | Motif involved in carotenoid and | 0.85 | 774 | 784 − 1.000 | 0.980 | aaATCTaaata | |
| | CTA.01 | tocopherol biosynthesis and in the | | | | | | |
| | | expression of photosynthesis-related genes | | | | | | |
| SEQ_12 | P$CCAF/CC | Circadian clock associated 1 | 0.85 | 777 | 791 − 1.000 | 0.868 | acatatga- | |
| | A1.01 | | | | | | AATCtaa | |
| SEQ_12 | P$OPAQ/02_ | Recognition site for BZIP transcription | 0.81 | 780 | 796 + 0.756 | 0.826 | gatttcATATgtt- | |
| | GCN4.01 | factors that belong to the group of | | | | | taat | |
| | | Opaque-2 like proteins | | | | | | |
| SEQ_12 | P$DOFF/PB | Prolamin box, conserved in cereal seed | 0.75 | 802 | 818 − 0.776 | 0.805 | tattttgtAAATa- | |
| | OX.01 | storage protein gene promoters | | | | | gaaa | |
| SEQ_12 | P$MADS/SQ | MADS-box protein SQUAMOSA | 0.90 | 821 | 840 + 1.000 | 0.950 | gacagctATTTt- | |
| | UA.01 | | | | | | tatatttaa | |
| SEQ_12 | P$TBPF/TAT | Plant TATA box | 0.88 | 826 | 840 − 1.000 | 0.945 | taaaTATAaa- | |
| | A.01 | | | | | | aatag | |
| SEQ_12 | P$GTBX/SB | SBF-1 | 0.87 | 831 | 847 + 1.000 | 0.880 | tttatatT- | |
| | F1.01 | | | | | | TAAttttgg | |
| SEQ_12 | P$SPF1/SP8 | DNA-binding protein of sweet potato that | 0.87 | 852 | 864 − 1.000 | 0.900 | acTACTgtga- | |
| | BF.01 | binds to the SP8a (ACTGTGTA) and SP8b | | | | | taa | |
| | | (TACTATT) sequences of sporamin and beta- | | | | | | |
| | | amylase genes | | | | | | |
| SEQ_12 | P$AREF/ | Silencing element binding factor- | 0.96 | 859 | 871 − 1.000 | 0.976 | accTGTCac- | |
| | BF.01 | transcriptional repressor | | | | | tact | |
| SEQ_12 | P$TALE/KN1_ | KNOTTED1 (KN1) and KNOTTED interacting | 0.88 | 862 | 874 + 1.000 | 0.982 | agtGACAggta- | |
| | KIP.01 | protein (KIP) are TALE class homeodomain | | | | ta | | |
| | | proteins. The KN1-KIP complex binds this | | | | | | |
| | | DNA motif with high affinity. | | | | | | |
| SEQ_12 | P$MYBL/GA | GA-regulated myb gene from barley | 0.91 | 887 | 903 + 1.000 | 0.929 | tttgttttGTTAact | |
| | MYB.01 | | | | | | tt | |
| SEQ_12 | P$MYBS/MY | MybSt1 (Myb Solanum tuberosum 1) with a | 0.90 | 899 | 915 − 1.000 | 0.943 | atagttATCCa- | |
| | BST1.01 | single myb repeat | | | | | gaaagt | |
| SEQ_12 | P$1BOX/GAT | Class I GATA factors | 0.93 | 902 | 918 + 1.000 | 0.935 | ttctgGATAac- | |
| | A.01 | | | | | | tataaa | |
| SEQ_12 | P$TBPF/TAT | Plant TATA box | 0.90 | 909 | 923 + 1.000 | 0.931 | taacTATAaat- | |
| | A.02 | | | | | | tatt | |
| SEQ_12 | P$AHBP/AT | Arabidopsis thaliana homeo box protein 1 | 0.90 | 915 | 925 + 1.000 | 0.989 | taaATTAtttg | |
| | HB1.01 | | | | | | | |
| SEQ_12 | P$AHBP/AT | Arabidopsis thaliana homeo box protein 1 | 0.90 | 915 | 925 − 0.789 | 0.900 | caaATAAttta | |
| | HB1.01 | | | | | | | |
| SEQ_12 | P$SPF1/SP8 | DNA-binding protein of sweet potato that | 0.87 | 942 | 954 − 0.777 | 0.872 | atAACTattgtga | |
| | BF.01 | binds to the SP8a (ACTGTGTA) and SP8b | | | | | | |
| | | (TACTATT) sequences of sporamin and beta- | | | | | | |
| | | amylase genes | | | | | | |
| SEQ_12 | P$LREM/AT | Motif involved in carotenoid and tocopherol | 0.85 | 950 | 960 − 1.000 | 0.922 | atATCTa- | |
| | CTA.01 | biosynthesis and in the expression of photosynthesis- | | | | taac | | |
| | | related genes | | | | | | |
| SEQ_12 | P$PSRE/GA | GAAA motif involved in pollen specific | 0.83 | 951 | 967 + 0.750 | 0.834 | ttataGATA- | |
| | AA.01 | transcriptional activation | | | | | tattctac | |
| SEQ_12 | P$MYBL/GA | GA-regulated myb gene from barley | 0.91 | 974 | 990 + 1.000 | 0.929 | tttgttttGTTAact | |
| | MYB.01 | | | | | | tt | |
| SEQ_12 | P$MYBS/MY | MybSt1 (Myb Solanum tuberosum 1) with a | 0.90 | 986 | 1002 − 1.000 | 0.943 | gtagttATCCa- | |
| | BST1.01 | single myb repeat | | | | | gaaagt | |
| SEQ_12 | P$1BOX/GAT | Class I GATA factors | 0.93 | 989 | 1005 + 1.000 | 0.935 | ttctgGATAac- | |
| | A.01 | | | | | | tacaaa | |
| SEQ_12 | P$MYBL/GA | GA-regulated myb gene from barley | 0.91 | 991 | 1007 − 1.000 | 0.957 | gatttgtaGT- | |
| | MYB.01 | | | | | | TAtccag | |
| SEQ_12 | P$AHBP/AT | Arabidopsis thaliana homeo box protein 1 | 0.90 | 1002 | 1012 − 0.789 | 0.900 | caaATGAtttg | |
| | HB1.01 | | | | | | | |
| SEQ_12 | P$AHBP/AT | HDZip class I protein ATHB5 | 0.89 | 1002 | 1012 + 0.936 | 0.936 | caaATCAtttg | |
| | HB5.01 | | | | | | | |
| SEQ_12 | P$NCS1/NC | Nadulin consensus sequence 1 | 0.85 | 1002 | 1012 − 0.878 | 0.904 | cAAATgatttg | |
| | S1.01 | | | | | | | |
| SEQ_12 | P$DOFF/PB | Prolamin box, conserved in cereal seed | 0.75 | 1003 | 1019 − 0.776 | 0.757 | tgatatgcAAAT- | |
| | OX.01 | storage protein gene promoters | | | | | gattt | |
| SEQ_12 | P$STKM/ST | Storekeeper (STK), plant specific DNA | 0.85 | 1016 | 1030 − 1.000 | 0.917 | cccTAAAa- | |
| | K.01 | binding protein important for tuber- | | | | | aattgat | |
| | | specific and sucrose-inducible gene | | | | | | |
| | | expression | | | | | | |
| SEQ_12 | O$RVUP/LT | Upstream element of C-type Long Terminal | 0.76 | 1024 | 1044 − 1.000 | 0.779 | tacagcac- | |
| | RUP.01 | Repeats | | | | | taaTTTCccta- | |
| | | | | | | | aa | |
| SEQ_12 | P$DOFF/PB | Prolamin box, conserved in cereal seed | 0.75 | 1036 | 1052 + 0.776 | 0.781 | agtgctgtA- | |
| | OX.01 | storage protein gene promoters | | | | | AATtttca | |
| SEQ_12 | P$GTBX/GT | GT1-Box binding factors with a trihelix | 0.85 | 1036 | 1052 + 0.968 | 0.881 | agtgctGTA- | |
| | 1.01 | DNA-binding domain | | | | | Aattttca | |
| SEQ_12 | P$CCAF/CC | Circadian clock associated 1 | 0.85 | 1051 | 1065 + 1.000 | 0.973 | caaaaaaa- | |
| | A1.01 | | | | | | ATCtat | |
| SEQ_12 | P$LREM/AT | Motif involved in carotenoid and | 0.85 | 1058 | 1068 + 1.0 | 0.897 | | |
| | CTA.01 | tocopherol biosynthesis andin the | | | | | aaATCTataga | |
| | | expression of photosynthesis-related genes | | | | | | |
| SEQ_12 | P$TBPF/TAT | Plant TATA box | 0.90 | 1059 | 1073 + 1.000 | 0.918 | aatcTATAga- | |
| | A.02 | | | | | | taatc | |
| SEQ_12 | P$LREM/AT | Motif involved in carotenoid and tocopherol | 0.85 | 1061 | 1071 − 1.000 | 0.897 | ttATCTataga | |
| | CTA.01 | biosynthesis and in the expression of | | | | | | |
| | | photosynthesis-related genes | | | | | | |
| SEQ_12 | P$CCAF/CC | Circadian clock associated 1 | 0.85 | 1062 | 1076 + 1.000 | 0.858 | ctatagatAATC- | |
| | A1.01 | | | | | | tat | |
| SEQ_12 | P$L1BX/ATM | L1-specific homeodomain protein ATML1 | 0.82 | 1073 | 1089 − 0.750 | 0.833 | aaaccaT- | |
| | L1.01 | (A. thalian ameristem layer 1) | | | | | CAAtgcatag | |
| SEQ_12 | 0$RPOA/P0 | Mammalian C-type LTR Poly A signal | 0.76 | 1075 | 1095 − 1.000 | 0.801 | cataaTAAAc- | |
| | LYA.01 | | | | | | catcaatgcat | |
| SEQ_12 | P$EINL/TEIL | TEIL (tobacco EIN3-like) | 0.92 | 1093 | 1101 + 0.964 | 0.922 | | |
| | .01 | | | | | | aTGAAcata | |
| SEQ_12 | P$DOFF/DO | Dof1/MNB1a-single zinc finger | 0.98 | 1107 | 1123 − 1.000 | 0.984 | taactagtAAA- | |
| | F1.01 | transcription factor | | | | | Gaatga | |
| SEQ_12 | P$GTBX/SB | SBF-1 | 0.87 | 1114 | 1130 + 1.000 | 0.888 | ttactagTTAAa- | |
| | F1.01 | | | | | | tatta | |
| SEQ_12 | P$WBXF1WR | WRKY plant specific zinc-finger-type | 0.92 | 1189 | 1205 + 1.000 | 0.978 | ttgctTTGAcca- | |
| | KY.01 | factor associated with pathogen defence, | | | | | aaaaa | |
| | | W box | | | | | | |
| SEQ_12 | P$OCSE/OC | OCS-like elements | 0.69 | 1203 | 1223 + 0.807 | 0.692 | aaaaagaattgc- | |
| | SL.01 | | | | | | taACATgta | |
| SEQ_12 | P$OPAQ/02_ | Recognition site for BZIP transcription | 0.81 | 1211 | 1227 + 1.000 | 0.882 | ttgctaACATg- | |
| | GCN4.01 | factors that belong to the group of | | | | | | tatcaa |
| | | Opaque-2 like proteins | | | | | | |
| SEQ_12 | P$MADS/AG | AGL2, Arabidopsis MADS-domain protein | 0.82 | 1219 | 1239 − 0.869 | 0.822 | tacatCCA- | |
| | L2.01 | AGAMOUS-like 2 | | | | | Gattttgatacat | |
| SEQ_12 | P$CCAF/CC | Circadian clock associated 1 | 0.85 | 1220 | 1234 + 1.000 | 0.899 | tgtatcaa- | |
| | A1.01 | | | | | | AATCtgg | |
| SEQ_12 | P$OCSE/OC | OCS-like elements | 0.69 | 1233 | 1253 + 0.807 | 0.719 | ggatgtatggata- | |
| | SL.01 | | | | | | tACATatc | |
| SEQ_12 | P$MYBS/TA | MYB protein from wheat | 0.83 | 1234 | 1250 − 1.000 | 0.896 | atgtATATcca- | |
| | MYB80.01 | | | | | | tacatc | |
| SEQ_12 | P$MYBS/TA | MYB protein from wheat | 0.83 | 1239 | 1255 + 1.000 | 0.905 | atggATATaca- | |
| | MYB80.01 | | | | | | tatctt | |
| SEQ_12 | P$AHBP/AT | Arabidopsis thaliana homeo box protein 1 | 0.90 | 1256 | 1266 − 1.000 | 0.989 | | |
| | HB1.01 | | | | | | gtaATTAttat | |
| SEQ_12 | P$AHBP/HA | Sunflower homeodomain leucine-zipper | 0.87 | 1256 | 1266 + 1.000 | 0.957 | ataataAT- | |
| | HB4.01 | protein Hahb-4 | | | | | TAc | |
| SEQ_12 | P$GTBX/GT | GT1-Box binding factors with a trihelix | 0.85 | 1256 | 1272 − 0.968 | 0.923 | actttgGTAAt- | |
| | 1.01 | DNA-binding domain | | | | | tattat | |
| SEQ_12 | P$GTBX/GT | Trihelix DNA-binding factor GT-3a | 0.83 | 1265 | 1281 − 1.000 | 0.889 | aggtatGT- | |
| | 3A.01 | | | | | | TActttggt | |
| SEQ_12 | P$MYBS/OS | Rice MYB proteins with single DNA binding | 0.82 | 1280 | 1296 − 1.000 | 0.825 | tttcgTATC- | |
| | MYBS.01 | domains, binding to the amylase element | | | | | tatgtgag | |
| | | (TATCCA) | | | | | | |
| SEQ_12 | P$GARP/AR | Type-B response regulator (ARR10), member | 0.97 | 1287 | 1295 + 1.000 | 0.985 | AGATacgaa | |
| | R10.01 | of the GARP- family of plant myb-related | | | | | | |
| | | DNA binding motifs | | | | | | |
| SEQ_12 | P$NCS1/NC | Nodulin consensus sequence 1 | 0.85 | 1293 | 1303 + 1.000 | 0.893 | gAAAAgcttac | |
| | S1.01 | | | | | | | |
| SEQ_12 | P$WBXF1WR | WRKY plant specific zinc-finger-type | 0.92 | 1298 | 1314 − 1.000 | 0.975 | aatttTTGActg- | |
| | KY.01 | factor associated with pathogen defence, | | | | | taagc | |
| | | W box | | | | | | |
| SEQ_12 | P$NCS2/NC | Nodulin consensus sequence 2 | 0.79 | 1312 | 1326 − 0.750 | 0.812 | tagtgtATCTt- | |
| | S2.01 | | | | | | gaat | |
| SEQ_12 | P$GBOX/TG | Arabidopsis leucine zipper protein TGA1 | 0.90 | 1325 | 1345 − 1.000 | 0.982 | gagggcT- | |
| | A1.01 | | | | | | GACgtttttaggta | |
| SEQ_12 | P$GBOX/HB | Wheat bZIP transcription factor HBP1B | 0.83 | 1326 | 1346 + 1.000 | 0.938 | acctaaa- | |
| | P1B.01 | (histone gene binding protein 1b) | | | | | aACGT- | |
| | | | | | | | cagccctct | |
| SEQ_12 | P$GBOX/BZ1 | bZIP transcription factor from Antirrhinum | 0.84 | 1331 | 1351 − 1.000 | 0.952 | ttgtaagagggcT- | |
| | P910.02 | majus | | | | | GACgtttt | |
| SEQ_12 | P$OCSE/OC | OCS-like elements | 0.69 | 1331 | 1351 − 1.000 | 0.727 | ttgtaagagggct- | |
| | SL.01 | | | | | | gACGTttt | |
| SEQ_12 | P$LREM/AT | Motif involved in carotenoid and | 0.85 | 1367 | 1377 + 1.000 | 0.882 | ccATCTata- | |
| | CTA.01 | tocopherol biosynthesis and in the | | | | | ta | |
| | | expression of photosynthesis-related genes | | | | | | |
| SEQ_12 | 0$LTUP/TA | Lentiviral TATA upstream element | 0.71 | 1382 | 1404 + 1.000 | 0.769 | cttgactatca- | |
| | ACC.01 | | | | | | gAACCtcaaaat | |
| SEQ_12 | P$OCSE/OC | OCS-like elements | 0.69 | 1393 | 1413 + 0.769 | 0.701 | gaacctcaaaat- | |
| | SL.01 | | | | | | taACTTctc | |
| SEQ_12 | P$GTBX/SB | SBF-1 | 0.87 | 1398 | 1414 − 1.000 | 0.883 | tgagaagT- | |
| | F1.01 | | | | | | TAAttttga | |
| SEQ_12 | P$PSRE/GA | GAAA motif involved in pollen specific | 0.83 | 1411 | 1427 − 1.000 | 0.896 | tctgaGA- | |
| | | transcriptional activation | | | | | AAtgtttgag | |
| SEQ5_12 | P$GCCF/ER | Ethylene-responsive elements (ERE) and | 0.85 | 1450 | 1462 + 1.000 | 0.924 | aagagtCGC- | |
| | E_JERE.01 | jasmonate-and elicitor-responsive elements | | | | | Caag | |
| | | (JERE) | | | | | | |

All references cited in this specification are herewith incorporated by reference with respect to their entire disclosure content and the disclosure content specifically mentioned in this specification.

The Figures Show:

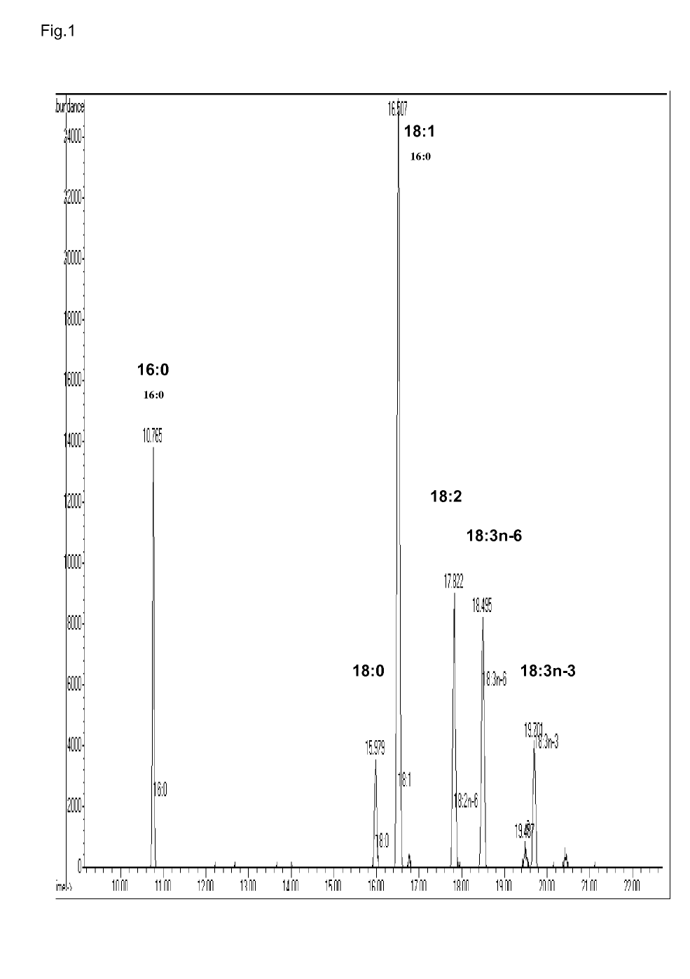

FIG. 1: Gas chromtogram of a transgenic line transformed with binary vector pSUN-BN3.

The invention will now be illustrated by the following Examples which are not intended, whatsoever, to limit the scope of this application.

### EXAMPLE 1

### General Cloning Methods

General Cloning Methods including enzymatic digestion by restriction enzymes, agarous gel electrophoresys, purification of DNA fragments, transfer of nucleic acids to nitrocellulose on nylon membranes, maligation of DNA fragments, transformation of E. coli bacteria as well as culture of bacteria and sequence analysis of recombinant DNA have been carried out as described in Sambrook et al. (1989, Cold Spring Harbour Laboratory Press. ISBN 0-87969-309-6).

### EXAMPLE 2

### Cloning of Promotor Elements from Brassica napus

For the analysis of potentially seeds specific expressed genes in Brassica napus, three different seed stages have been investigated. To this end, Brassica napus cv. Westar plants were raised under standard conditions (Moloney et al. 1992, Plant Cell Reports 8: 238-242). The seeds have been harvested 20 days, 25 days and 40 days after flowering. The seeds were used for the preparation of RNA (RNAeasy, Qiagen) according to the manufactures manual. In parallel, plant material from roots, leaves and stipes has been used for preparation of RNA. The said RNA was mixed and used as a control for the further experiments. RNA from the seed stages as well as control RNA were treated by the one-color gene expression kit (Agilent) for microarray-analysis. The Arapidopsis whole genome chip (Agilent) was hybridized with the treated RNA. Based on different labelled RNAs, the genes from Brassica napus could be identified which are expressed in the seeds solely but not in other organs or tissues. Six genes from Arabidopsis thaliana have been identified which hybridized with the probes from Brassica napus (Table 7).

TABLE 7
Arabidopsis genes which were capable of hybridizing
the seeds specific probes from Brassica napus:

| Arabidopsis sequence | Protein function | Expression pattern |
|---|---|---|
| At1g23200 | pectinesterase | seed |
| At1g52690 | LEA gene | seed |
| At1g61720 | anthocyanidin reductase | seed |
| At2g38900 | Proteinase inhibitor | seed |
| At3g15670 | LEA gene | seed |
| At5g38170 | Lipid transfer protein | seed |

Based on the gene sequences from Arabidopsis thaliana, homologs have been identified in Brassica napus cDNA libraries. For all six Arabidopsis genes, homologous coding sequences in Brassica napus could be identified (Table 8).

TABLE 8
Homology sequence from Brassica napus corresponding
to the Arabidopsis sequence shown in Table 7.

| Brassica napus | | Arabidopsis | |
|---|---|---|---|
| sequence | SEQ ID NO: | homolog | Protein function |
| BN1 | 1 | At1g23200 | pectinesterase |
| BN2 | 2 | At1g52690 | LEA gene |
| BN3 | 3 | At1g61720 | anthocyanidin |
| | | | reductase |
| BN4 | 4 | At2g38900 | Proteinase inhibitor |
| BN6 | 5 | At3g15670 | LEA gene |
| BN8 | 6 | At5g38170 | Lipid transfer protein |

From leaf material of Brassica napus cv. Westar, genomic DNA has been isolated using the DNAeasy kit (Qiagen) according to the manufacturer's manual. Culture conditions for the Brassica napus cv. Westar were as discussed above. Based on the genomic DNA, a genomic DNA library was established using the Genome Walker kit (Clontech). The following primer sequences were derived from Brassica napus cDNA sequences in order to isolate upstream sequences of the Brassica napus genomic sequences (Table 9).

TABLE 9
Primer sequences for the implication
of 5 prime upstream sequences in com-
bination with AP1/ AP2 (Clontech).

| Brassica napus | | |
|---|---|---|
| sequence | Primer sequence 5′-3′ | SEQ ID NO: |
| BN1 | ATTGGTATAATATATTTGG | 14 |
| BN2 | GTTTCTGTGTAGAGAAACTG | 15 |
| BN3 | CTGATTAAATTCTTAAGACCAG | 16 |
| BN4 | CCAAAATTACCAG CACATTC | 17 |
| BN6 | GTTGCTGTGTATAAACTGTG | 18 |
| BN8 | TCTGAGAAATGTTTGAGAAG | 19 |

The indicated primers were used in combination with the AP1 and AP2 primers according to the manufacturer's manual of the Genome Walker kit in a PCR. PCR conditions were as follows:

Primary PCR:

7 cycles 94° C., 25 seconds, 72° C., 4 minutes,

32 cycles 94° C., 25 seconds 67° C. 4 minutes,

final cycle 67° C., 4 minutes.

Secondary PCR

5 cycles 94° C., 25 seconds, 72° C. 4 minutes,

22 cycles 94° C., 25 seconds, 67° C., 4 minutes,

final cycle 67° C., 4 minutes.

Using the former specific primers, specific fragments for the primer combinations were obtained. These fragments were cloned into the pGEM-T (Pomega) vector using the manufacturer's manual and sequenced by standard techniques (laser fluorescent DNA-sequenceing, ABI according to the method of Sanger et al. 1977 Proc. Natl. Acad. Sci. USA 74, 5463-5467). The following Brassica napus sequences have been obtained (Table 10).

TABLE 10
Genomic five prime upstream sequences from
the Brassica napus cDNA sequences.

| Brassica napus | genomic 5′ sequence in | |
|---|---|---|
| Sequence | bp | SEQ ID NO: |
| BN1 | 1336 | 7 |
| BN2 | 1532 | 8 |
| BN3 | 1612 | 9 |
| BN4 | 1767 | 10 |
| BN6 | 2281 | 11 |
| BN8 | 1490 | 12 |

The analysis of the 5′ upstream sequences using Genomatix software GemsLauncher showed that the sequences comprised promoter elements. This was confirmed by the presence of a TATA-Box which is required for transcription by RNA-polymerases. Also in the isolated fragments elements specific for seed-transcription factors (e.g. Prolamin-box, legumin box, RITA etc.) were found.

### EXAMPLE 3

### Production of Test Constructs for Demonstrating Promoter Activity

For the testing of the promoter elements in a first step promoter terminator cassettes were generated. To this end, fusion PCRs have been used wherein via two PCR steps a CaMV35S terminator was linked with promoter elements. In a further step, a multiple cloning site was introduced in between the promoter and terminator elements. The primers used are shown in Table 11.

TABLE 11

Primer pairs used for the generation of
promoter-terminated-cassettes via Fusion-PCR.

| Brassica napus | | | |
|---|---|---|---|
| Promoter/Termin | Primer pair 1. | Primer pair 1. | Primer pair 2. |
| ator cassette | PCR Promoter | PCR Terminator | PCR |
| p-BN1_t-35S | 5′- | 5′- | 5′- |
| | acctgcaggttaggccggc- | ccatggacttaggccttagcttaat- | acctgcaggttaggccggc- |
| | cacttgtcatatatatatgac | taactaagtcgacaagctc- | cacttgtcatatatatatgac |
| | (SEQ ID NO: 20) | gagtttctccataataatg | (SEQ ID ID NO:24) |
| | 3′- | (SEQ ID NO: 22) | 3′gaattaattcggcgttaattcaggg |
| | tcacaaacctcccgatgtttataca- | 3′gaattaattcggcgttaattcaggg | cgcc |
| | caccatggacttaggccttagct- | cgcc | (SEQ ID NO: 25) |
| | taattaactaagtcgacaagctc- | (SEQ ID NO: 23) | |
| | gag | | |
| | (SEQ ID NO: 21) | | |
| p-BN2_t-35S | 5′- | 5′- | 5′- |
| | acctgcaggttaggccggcca- | ccatggacttaggccttagcttaat- | acctgcaggttaggccggcca- |
| | taaccctctccatgttgatac | taactaagtcgacaagctc- | taaccctctccatgttgatac |
| | (SEQ ID NO: 26) | gagtttctccataataatg | (SEQ ID NO: 30) |
| | 3′- | (SEQ ID NO: 28) | 3′gaattaattcggcgttaattcaggg |
| | cctttgaagaaaagaaaccatg- | 3′gaattaattcggcgttaattcaggg | cgcc |
| | gacttaggccttagcttaattaac- | cgcc | (SEQ ID NO: 31) |
| | taagtcgacaagctcgag | (SEQ ID NO: 29) | |
| | (SEQ ID NO: 27) | | |
| p-BN3_t-35S | 5′- | 5′- | 5′- |
| | acctgcaggttaggccggccacta- | ccatggacttaggccttagcttaat- | acctgcaggttaggccggccacta- |
| | tagggcacgcgtggtcg | taactaagtcgacaagctc- | tagggcacgcgtggtcg (SEQ ID |
| | (SEQ ID NO: 32) | gagtttctccataataatg | (SEQ ID NO: 36) |
| | 3′- | (SEQ ID NO: 34) | 3′gaattaattcggcgttaattcaggg |
| | gcgttaagaatttataatatatcagc- | 3′gaattaattcggcgttaattcaggg | cgcc |
| | catggacttaggccttagcttaat- | cgcc | (SEQ ID NO: 37) |
| | taactaagtcgacaagctcgag | (SEQ ID NO: 35) | |
| | (SEQ ID NO: 33) | | |
| p-BN4_t-35S | 5′- | 5′- | 5′- |
| | acctgcaggttaggccggccgtt- | ccatggacttaggccttagcttaat- | acctgcaggttaggccggccgtt- |
| | gatggaaatcgtatcgtcg | taactaagtcgacaagctc- | gatggaaatcgtatcgtcg (SEQ |
| | (SEQ ID NO: 38) | gagtttctccataataatg | (SEQ ID ID NO: 42) |
| | 3′- | (SEQ ID NO: 40) | 3′gaattaattcggcgttaattcaggg |
| | ctgcaaagataaaaaaaaagggg- | 3′gaattaattcggcgttaattcaggg | cgcc |
| | tagcaacccatggacttaggcct- | cgcc | (SEQ ID NO: 41) |
| | tagcttaattaactaagtcga- | (SEQ ID NO: 41) | |
| | caagctcgag | | |
| | (SEQ ID NO: 39) | | |
| p-BN6_t-35S | 5′- | 5′- | 5′- |
| | acctgcaggttaggccggccttg- | ccatggacttaggccttagcttaat- | acctgcaggttaggccggccttg- |
| | tactctcccttaatggag | taactaagtcgacaagctc- | tactctcccttaatggag (SEQ ID |
| | (SEQ ID NO: 44) | gagtttctccataataatg | (SEQ ID NO:48) |
| | 3′- | (SEQ ID NO: 46) | 3′gaattaattcggcgttaattcaggg |
| | cctatttgcgcatttgaagaaagaa- | 3′gaattaattcggcgttaattcaggg | cgcc |
| | aaccatggacttaggccttagct- | cgcc | (SEQ ID NO: 49) |
| | taattaactaagtcgacaagctc- | (SEQ ID NO: 47) | |
| | gag | | |
| | (SEQ ID NO: 45) | | |
| p-BN8_t-35S | 5′- | 5′- | 5′- |
| | acctgcaggttaggccggccgt- | ccatggacttaggccttagcttaat- | acctgcaggttaggccggccgt- |
| | gaatcacaagcaaagag | taactaagtcgacaagctc- | gaatcacaagcaaagag (SEQ |
| | (SEQ ID NO:50) | gagtttctccataataatg | (SEQ ID ID NO: 54) |
| | 3′- | (SEQ ID NO: 52) | 3′gaattaattcggcgttaattcaggg |
| | gagtcgccaagcttacaaaacc- | 3′gaattaattcggcgttaattcaggg | cgcc |
| | catggacttaggccttagcttaat- | cgcc | (SEQ ID NO: 55) |
| | taactaagtcgacaagctcgag | (SEQ ID NO: 53) | |
| | (SEQ ID NO: 51) | | |

The promoter-terminator cassettes were cloned into the pGEMT (Promega vector) according to the manufacturer's manual and subsequently sequenced. Via the restriction site of Sbf1-EcoRV (New England Biolabs), cassettes were transferred into the vector pENTRB (Invitrogen) according to standard techniques. In a further step, the delta 6

Desaturase Gene (SEQ ID NO: 13) was introduced via the Nco1-Pac1 restriction sites into the generated pENTRB vectors pENTRB-p-BN1_t-35S, pENTRB-p-BN2_t-35S, pENTRB-p-BN3_t-35S, pENTRB-p-BN4_t-35S, pENTRB-p-BN6_t-35S, pENTRB-p-BN8₁₃ t-35S.

The resulting vectors were subsequently used for Gateway (Invitrogen) reactions together with the binary plasmid pSUN to generate binary vectors for the production of transgenic plants. The promoter activity in the transgenic plant seeds was measured based on the expression of delta 6 Desaturase and an observed modification in the lipid pattern of the seeds.

### EXAMPLE 4

### Production of Transgenic Plants

a) Generation of Transgenic Rape Seed Plants (Amended Protocol According to Moloney et al. 1992, Plant Cell Reports, 8:238-242)

For the generation of transgenic rapeseed plants, the binary vectors were transformed into Agrobacterium tumefaciens C58C1:pGV2260 (Deblaere et al. 1984, Nucl. Acids. Res. 13: 4777-4788). For the transformation of rapeseed plants (Var. Drakkar, NPZ Norddeutsche Pflanzenzucht, Hohenlieth, Deutschland) a 1:50 dilution of an overnight culture of positive transformed acrobacteria colonies grown in Murashige-Skoog Medium (Murashige and Skoog 1962 Physiol. Plant. 15, 473) supplemented by 3% saccharose (3MS-Medium) was used. Petiols or Hypocotyledones of sterial rapeseed plants were incubated in a petri dish with a 1:50 acrobacterial dilusion for 5-10 minutes. This was followed by a tree day co-incubation in darkness at 25° C. on 3MS-Medium with 0.8% bacto-Agar. After three days the culture was put on to 16 hours light/8 hours darkness weekly on MS-medium containing 500 mg/l Claforan (Cefotaxime-Natrium), 50 mg/l Kanamycine, 20 mikroM Benzylaminopurin (BAP) and 1.6 g/l Glucose. Growing sprouts were transferred to MS-Medium containing 2% saccharose, 250 mg/l Claforan and 0.8% Bacto-Agar. Even after three weeks no root formation was observed, a growth hormone 2-Indolbutyl acid was added to the medium for enhancing root formation.

Regenerated sprouts have been obtained on 2MS-Medium with Kanamycine and Claforan and were transferred to the green house for sprouting. After flowering, the mature seeds were harvested and analysed for expression of the Desaturase gene via lipid analysis as described in Qui et al. 2001, J. Biol. Chem. 276, 31561-31566.

b) Production of Transgenic Flax Plants

The production of transgenic flax plants can be carried out according to the method of Bell et al., 1999, In Vitro Cell. Dev. Biol. Plant 35(6):456-465 using particle bombardment. Acrobacterial transformation could be carried out according to Mlynarova et al. (1994), Plant Cell Report 13: 282-285.

c) Production of Transgenic Arabidopsis Plants

Transgenic Arabidopsis plants were generated according to the protocol of Bechthold et al. 1993 (Bechthold, N., Ellis, J., Pelletier, G. (1993) In planta Agrobacterium-mediated gene transfer by infiltration of Arabidopsis thaliana plants. C.R. Acad. Sci. Ser. III Sci. Vie., 316, 1194-1199). Arabidopsis plants of the ecotype Col0fae1 were grown on soil after a vernalisation of the seeds for 3 days at 4° C. After plants started to flower, they were dipped into an Agrobacterium tumefaciens solution containing Agrobacterium strain pMP90 transformed with the binary plamsids as described in Example 3 and following other components: ½ MS pH 5.7, 5% (w/v) Sacharose, 4.4 μM Benzylaminopurin, 0.03% Silwet L-77 (Lehle Seeds, Round Rock, Tex., USA). Agrobacterium solution was diluted to a final concentration of OD₅₄ 0.8. Plants were dipped two times into above described solution and keep 4-6 weeks for normal growth and seed formation. Dried seeds were harvested and and subjected to selective growth based on the tolerance against the herbicide Pursuit (BASF). Seeds of this generation of selected plants were then subjected to lipid analysis.

### EXAMPLE 5

### Lipid Extraction

Lipids can be extracted as described in the standard literature including Ullman, Encyclopedia of Industrial Chemistry, Bd. A2, S. 89-90 und S. 443-613, VCH: Weinheim (1985); Fallon, A., et al., (1987) “Applications of HPLC in Biochemistry” in: Laboratory Techniques in Biochemistry and Molecular Biology, Bd. 17; Rehm et al. (1993) Bio-technology, Bd. 3, Kapitel III: “Product recovery and purification”, S. 469-714, VCH: Weinheim; Belter, P. A., et al. (1988) Bioseparations: downstream processing for Bio-technology, John Wiley and Sons; Kennedy, J. F., und Cabral, J. M. S. (1992) Recovery processes for biological Materials, John Wiley and Sons; Shaeiwitz, J. A., und Henry, J. D. (1988) Biochemical Separations, in: Ullmann's Encyclopedia of Industrial Chemistry, Bd. B3; Kapitel 11, S. 1-27, VCH: Weinheim; und Dechow, F. J. (1989) Separation and purification techniques in biotechnology, Noyes Publications.

Alternatively, extraction will be carried out as described in Cahoon et al. (1999) Proc. Natl. Acad. Sci. USA 96 (22):12935-12940, und Browse et al. (1986) Analytic Biochemistry 152:141-145. Quantitative and qualitative analysis of lipids or fatty acids are described in Christie, William W., Advances in Lipid Methodology, Ayr/Scotland: Oily Press (Oily Press Lipid Library; 2); Christie, William W., Gas Chromatography and Lipids. A Practical Guide—Ayr, Scotland: Oily Press, 1989, Repr. 1992, IX, 307 S. (Oily Press Lipid Library; 1); “Progress in Lipid Research, Oxford: Pergamon Press, 1 (1952)-16 (1977) u.d.T.: Progress in the Chemistry of Fats and Other Lipids CODEN.

Based on the analysed lipids, the expression of the Desaturase were determined since the lipid pattern of successfully transformed plant seeds are differing from the pattern of control plant seeds.

Analysis of Promoter BN3:

Seeds from different Arabidopsis plants containing the T-DNA of binary vector pSUN-BN3 (see Example 3) were subjected to lipid analysis as described above (Tab. 12 and FIG. 1). Compared to the non-transgenic control plants (WT), plants containing pSUN-BN3 produced in addition to the fatty acids found in the control plants a novel fatty acid, γ-linolenic acid (18:3Δ6,9,12). The synthesis of this novel fatty acid is subject to the enzyme Δ6-desaturase, which gene is behind the BN3 promoter. The fact that the novel fatty acid can be detected in significant amounts in the seeds of Arabidopsis plants containing the T-DNA of binary vector pSUN-BN3 is explained by the functional expression of the gene Δ6-desaturase from Pythium irregulare. According to the identification of the novel generated fatty acid γ-linolenic acid, the promoter BN3 is enabling the functional expression of the respective gene.

Therefore the promoter BN3 is a functional promoter, driving expression of genes in seeds.

Other parts of the transgenic Arabidopsis plants were also subjected to gas chromatographic analysis, but no other fatty acids than in the non-transgenic Arabidopsis plants were observed.

Therefore, the promoter BN3 is driving functional expression in a seed-specific manner, thereby only allowing the transcription of attached genes in seeds.

TABLE 12
Gas chromatographic analysis of seeds from different
Arabidopsis plants either being non-transgenic controls
(WT) or transgenic lines containing the T-DNA of plasmid
pSUN-BN3 derived from chromatograms as shown in FIG. 1.
The respective fatty acids are given in chemical nomeclature
(16:0 palmitic acid, 18:0 stearic acid, 18:1-n-9 oleic acid,
18:2n-6 linoleic acid, 18:3n-6 γ-linolenic acid,
18:3n-3 α-linolenic acid). The fatty acid 18:3n-6 is a
product of the enzymatic reaction of the Δ6-
desaturase from Pyhtium irregulare, which is not
observed in the non-transgenic control lines.

| sample name | 16:0 | 18:0 | 18:1n-9 | 18:2n-6 | 18:3n-6 | 18:3n-3 |
|---|---|---|---|---|---|---|
| WT | | | | | | |
| WT | 13.03 | 3.68 | 27.65 | 35.37 | 0.00 | 20.27 |
| WT | 14.04 | 3.51 | 25.16 | 42.81 | 0.00 | 14.49 |
| WT | 9.63 | 2.66 | 36.85 | 35.07 | 0.00 | 15.80 |
| WT | 10.16 | 2.80 | 37.84 | 35.35 | 0.00 | 13.86 |
| WT | 8.94 | 3.02 | 31.66 | 36.93 | 0.00 | 19.44 |
| WT | 9.52 | 2.93 | 29.74 | 37.15 | 0.00 | 20.66 |
| pSUN-BN3_1 | 13.19 | 2.82 | 35.20 | 25.31 | 12.20 | 11.28 |
| pSUN-BN3_2 | 14.23 | 5.25 | 44.38 | 14.58 | 14.57 | 7.00 |
| pSUN-BN3_3 | 13.27 | 3.45 | 46.13 | 18.62 | 9.86 | 8.67 |
| pSUN-BN3_4 | 11.76 | 2.08 | 43.63 | 29.17 | 3.97 | 9.39 |
| pSUN-BN3_5 | 12.26 | 1.76 | 42.01 | 25.92 | 8.12 | 9.94 |
| pSUN-BN3_6 | 10.25 | 3.22 | 35.82 | 26.64 | 10.43 | 13.65 |
| pSUN-BN3_7 | 10.65 | 3.78 | 34.04 | 23.74 | 14.62 | 13.17 |

1. A polynucleotide comprising an expression control sequence which allows seed specific expression of a nucleic acid of interest being operatively linked thereto, said expression control sequence being selected from the group consisting of:
(a) an expression control sequence having a nucleic acid sequence as shown in any one of SEQ ID NOs: 7 to 12;
(b) an expression control sequence having a nucleic acid sequence which hybridizes under stringent conditions to a nucleic acid sequence as shown in any one of SEQ ID NOs: 7 to 12;
(c) an expression control sequence having a nucleic acid sequence which hybridizes to a nucleic acid sequences located upstream of an open reading frame sequence shown in any one of SEQ ID NOs: 1 to 6;
(d) an expression control sequence having a nucleic acid sequence which hybridizes to a nucleic acid sequences located upstream of an open reading frame sequence being at least 80% identical to an open reading frame sequence as shown in any one of SEQ ID NOs: 1 to 6;
(e) an expression control sequence obtainable by 5′ genome walking from an open reading frame sequence as shown in any one of SEQ ID NOs: 1 to 6; and
(f) an expression control sequence obtainable by 5′ genome walking from an open reading frame sequence being at least 80% identical to an open reading frame as shown in any one of SEQ ID NOs: 1 to 6.

2. The polynucleotide of claim 1, wherein said expression control sequence comprises at least 1,000 nucleotides.

3. The polynucleotide of claim 1, wherein said polynucleotide further comprises a nucleic acid of interest being operatively linked to the expression control sequence.

4. The polynucleotide of claim 1, wherein said polynucleotide further comprises a termination sequence which allows for termination of the transcription of a nucleic acid of interest.

5. A vector comprising the polynucleotide of claim 1.

6. The vector of claim 5, wherein said vector is an expression vector.

7. A host cell comprising the polynucleotide of claim 1 or a vector comprising said polynucleotide.

8. The host cell of claim 7, wherein said host cell is a plant cell.

9. A non-human transgenic organism comprising the polynucleotide of claim 1 or a vector comprising said polynucleotide.

10. The non-human transgenic organism of claim 9, wherein said organism is a plant or a seed thereof.

11. A method for expressing a nucleic acid of interest in a host cell comprising
(a) introducing the polynucleotide of claim 1 or a vector comprising said polynucleotide into a host cell, whereby the nucleic acid sequence of interest will be operatively linked to the expression control sequence; and
(b) expressing said nucleic acid sequence in said host cell.

12. The method of claim 11, wherein said host cell is a plant cell.

13. A method for expressing a nucleic acid of interest in a non-human organism comprising
(a) introducing the polynucleotide of claim 1 or a vector comprising said polynucleotide into a non-human organism, whereby the nucleic acid sequence of interest will be operatively linked to the expression control sequence; and
(b) expressing said nucleic acid sequence in said non-human transgenic organism.

14. The method of claim 13, wherein said non-human transgenic organism is a plant or seed thereof.

15. The method of claim 13, wherein said nucleic acid of interest is expressed seed-specific.

16-17. (canceled)

18. The polynucleotide of any one of claim 1, wherein said nucleic acid of interest encodes a seed storage protein or is involved in the modulation of seed storage compounds.


SEQUENCE LISTING (13 sequences)

SEQ ID NO: 1 - DNA, 1888 bp, Brassica napus
atggatcacaaacctcccagatgtttatacaccaaatatattataccaataatttacgtcctatcaatttctcataccaacgcccatttcattacctcatgcaaacaaactccataccctaacgtctgcgcccaccacatatctaattcacctctcaacaccttagactaccaaaccgatgggctcgctttccacgaccttgtggttagctcgaccatggaccaggccgttcacctacaccgcctcgtctctaaggtgaaacggcgtcgttctttccacaaacatgccaagtcagctttgctcgactgcttggagctttacgaagacaccatagaccaactcaaccactctcggagatcatatgattttaacttttcagcgcatgatagacaaacctcgttaagcgctgccatagctaatcaagacacttgcaaaaacgggtttaaagattttaatctaacgtcttcctattcaaaatatttccctatacacgttcaccggaacctcactaaatcgataagcaactctctggcggtttcaaaggccgcggctgaggcggttgccgagaaacatccagggacagcgttcacaaaattcaacaagcaaggaagtagcggcggaggaggtggtggtggtggtcggagattgatgttttgggacacccaatttccttcgtggatccctcttaccgaccgtaaacttctggaaggttcagtaaccacggctaaacccgatcttgtggtagctaaagacggttcgggtcactacacgagtattcaacaggcgataaacgcagcagccaaacttcctaggagaaacaagagacttgtggtatacgttaaagccggcgtttaccgagaaaacgtggagatcaagaaatcagtcaagaacgtgatggttatcggtgacggcattgactctaccgtcgtcaccggtagtaggaacgttaaagatggcacgacgacgtttcggtccgcaacttttgctgttgccggcagcggttttatcgcacgagacataacattcgagaacactgcgggaccgcagaaacaccaagcggtggctctccgatcaggatcagatttcgccgtattctacggctgttctttcaaaggctaccaagacactctctaccttcactcccgccgtcagttcttgaaaaactgcaacgtctacggaaccgtcgacttcgtcttcggagacgcaaccgcagtcctccaaaactgcaacatctacgctcgtaaacccatgagcggacaaaaaaacactgtcacggctcagtcccgcaaggaccctaacgagaacacaggtttcgtcatccagagctcgacggtgtctacagcggcggagacgtacttaggaaggccgtggaagcagtattcgaggacggttttcatgaaatgcagtctcggagagatgctaaatccggcggggtggttgccttggagcggtgactttgctttgaggactctttattacggagaatatggtaacaccggcgccggagcaagtgtatccggtagagttaagtggccaggttatcatgttttgaaggcggcgactgaggcgggaaagttcacggtggagaatttcttggacgggaattattggattactgctgcgggagtgccggtcaatgctgggctttgactttgcggttcattaatcgtgactgtgcttgtggagtctttggtttttgttggcgtgttgatgtttatatgatatagttttattgtccatgtggattggatatggattgttttgaacatagaataagaaaatcaatgtaatgtggggtttggtcggaggaattggtatacatttatggtatactaggtaataacccgcctcttgcgcggaatgtgattattagtttcgttatttttaa

SEQ ID NO: 2 - DNA, 1428 bp, Brassica napus
gacacacagtttctctacacagaaacatacactttcaaaaaaagctttatataattatcgtttcatatttcacacatttgaaggggaaaaaatggcctccaaccaacaaagctacaaagctggtgaaactagaggcaagactcaggagaagacaggacaagctatgggagcaatgagggacaaggctgaggaaagcaagaacaagacttcccaagcggcccaaacagcccaacaaaaggcccatgagacggcacagtctgcaaaagataagacatctcaaactgcccaaaagacccagcaaaaggctgacgagacgacccaatcagcaaaaggcaagacatcccaagctgctcagacggctcaagacaaagcccgtgagacaaaggacaagacggggagctatatgtccgagacaggagaagccataaagcacaaggctcaagacgctgctcagtacacaaaggagacggctcaaggcgcggctcagtatacgaaagagactgctgaagctggtaaagacaagaccggtgggttcttaagccagactggtgagcatgtgaagcagatggctatgggtgcagctgatgcggtgaagcacacttttgggatggctaccgaggaagatgacaaggaacattttccaggcacaactactggtactactcggaccactgataccactcatcagacgtatcagaggaagtgagaataacaagagaactatgattgtgtctttctcttgtttctataatgtcgcatgtttggtcttttgagtttctgttgagtctttatgttttgtggttcagttttttttctttcgttgtgacggagacttctaatttccagccaataagtttagaatttgatcctgtcacaccttttgtgtcgaccaaattgtatttttattataataaaaagttatcaattccataccacgatttggattgtatgatgtgaaggagaactctgttattaaattacatgaaaggagtccctgaatctcctcagtgtgggtttagctcactggccgtaagatttctgttaatatatagtcagtttccaatgtatttttgtaattatatatggatatcctttgtttgaaactctgaatctaatgtgatgtgagttcttgtgttcagatgttccaatcaatgctagagacattcttgaagatcaagagttgaaaaacgctgtgaaatccttgtcttatcgttgtttccattgttttaccttatgatctcaagtcctcttgaaaaattataagaaggtgaactggaggagaaggttaagttatgctgactggtgaatgtattttttatttgcttttgtttactttcataagaatgtcttttgatgcacagcaataatgtctcaagaaactcaaaatctttggatctagtatgatcaagaccttgatatattgaagag

SEQ ID NO: 3 - DNA, 1005 bp, Brassica napus
gaaacaaacaaaatataaaatttctggtcttaagaatttaatcagatggcaaccgttgatcagaccgctgtaaccaccggaactaagaaggcttgtgtcatcggtggcacaggaaacttagcctctattctcatcaagcatttgcttcaaagcggctacaaagttaacaccacagttagagatccagaaaatgagaagaaaatggcacaccttaaggtacttcaagagcaaggggacctcaacatcttcaaggcggacttaaccgatgaagggagtttcaattcaccaatctcgggctgtgaatatgttttccatgtcgcaacaccaatcagctttacatctcaagatcccgagaaagacatgatcaaaccagcggtacaaggagtgatcaacgtgttgaaatcttgcttaaaatcgaactcaatcaagcgcgtgatctacacttcttcagctgctgcggtttctatcaacaacctttcggaacctggacttgtgatgaccgaagaaaactggtctgacgttgattttctcacaaaggagaagccgtttaactgggtaataacaatttcttgctgcacaagataggtttttttcccgactaagttcagttacctctctctgttttatttctagggttacccagtctcaaagactttagcagaaaaggaagcttataaatttgcggaagagaataagattgatctcgttactgtggttccagcactcatagccggaaactctctcctctctgatcctccgagcagtttatctctctcgatgtctttaatcactggtaaacatgaatcataatactatttgaccacttctgttaaagtttcacaatcaagatgattggtttttgttgttagggaaagaaatgcatctgagcggtctcaaggaaatgcagaagctatctggatccatctcgttcatccacgtggacgacctagctcgtgcacatatgtttcttgcggagaaagaaacagcttctggtcgctacatttg

SEQ ID NO: 4 - DNA, 784 bp, Brassica napus
atgtcgtccgaatgtgctggtaattttggttattgtatataatttgatagtgttacggctttaaatcaattctcttttatattgcttattcccggcccattcaaatcaagtcgaaaatcatttccggatttatctccttttgatactaacctaattgtctataggttgagcagactaaccgtacccttttaacctttctcaccatgcacgtgtaacaaggaatggtcgacctaactcaatagacaaaaccatgtgcattgaaactaaaacgttacttgttcttcttgctaaatagtagaaaccaattccatgcaatatttaccaacgattaatctagttggccaaattcaaatggacgtcatgtctattcgcttcttagtagaagtttaaacaaaatttctttgttttcaatcgtctagaaaataattaaatttatcataaatgtcgtaggagctgatacaaatcttgagaataatatgaatttgaagtctcttctataaaatccgttaatatatagatattgtatggccaagccatgctgatttttttttcccctcgccatgcatgttggtgtttcatttataacttcaaaatgttacttttaccgaatatgacctgggaggcatcaggcatggtctagggatcatattgttttaattagttccacggaaatcaatcttgagatggacttattgatctctaaaatgcaaataaattactagacgatctatatatctaatgtggttgctttgagtgcgtacagggaagaactcatggccggagc

SEQ ID NO: 5 - DNA, 1021 bp, Brassica napus
gacacagtttatacacagcaacaatcactttgagaagaagctttaattgtcgtttcatatttttacacatttgaagaaaagaaaaatggcgtctaaccaacaaagctacaaagctggtgaaaccagaggcaagactcaggagaagacaggacaagcaatgggagcaatgagggacaaggctgaggaaggcaaggacaagacttcccagacggctcaaaaggcccaacaaaaggcacaagagactgcccaggcagctaaagacaagacatctcaagctgcccaaacgacccaacaaaaggctcaagagacggctcaggcagcgaaagacaagacatctcaagctgcccaaacgactcagcaaaaggctcatgagacgacccaatcatcaaaagagaagacatctcaagctgcccagacggctcaagaaaaagcccgtgagacgaaggacaagaccgggagctacctgtcggagacaggtgaagccgtgaagcaaaaggctcaagacgcagctcagtacacaaaggagacggcgcaaaacgcggctcagtacacgaaagagacggctgaagcgggtaaagacaagaccggtgggttcttgagccagacaggtgagcatgtgaagcagatggctatgggtgcagctgatgcggtgaagcacacttttggaatggctacggaggaagaagacagggaacattatccaggaacaactacttgtactactcagagcactgatcagactcgtcatacttatgagaggaagtgagaataacaagaacaataaacgaacacctcttttcttttgtttcaataatgtcatgcgtctttgtttcgtaatttctgttgtgtccttgtgtttgtgattccgttttgtttctttcgttgtgatggtgatcctggagacttctaatttccagccaataagtttatattcgatcgtgttacaactttttttgtgtcgtccaatttgtatttttgttataataaactggtattaatcccaaaaaaaaaaaaaaaaaaa

SEQ ID NO: 6 - DNA, 497 bp, Brassica napus
gaacttctcaaacatttctcagaaaaaaaactagaactaaggttgaagagtcgccaagcttacaaaacatgtgttggaacccaataatcaacatgaagttcacaggagtagtatgcatcgcatttgtgatagtccttctgtcggctttagctccgaccaaagcagtctttgaagagaaagtggcatgcatcccgacagaacttatgacatgcataccagcattacaaaccggaagtcaaccgtccgctgaatgctgcggaaaactgaaagaacaagagtcgtgtttgtgtggttacatacaaaatccattgttttctcagtatgttacatctgaaaatgctcacaaagttttagcgacttgtggtataccttatcctacttgttaaaatttcaaatttcaaaatcctatgtaacaatccgtcatggtttatccattgatattaaataatcaatattttcttcatttttcttgtaaaaaaaaaaaaaaaaaaaaaaaa

SEQ ID NO: 7 - DNA, 1304 bp, Brassica napus
cttgtcatatatatatgacttccatagtttcttttggagttaaacgcatgactaattgttatgatttagctacgtgacagaaaattgcagatcaaagtgtcgttttttatagacctaaacacattaattaaaggataaagtaagaaaacttgtaacaacgaggaaacaaatgatttccacaaaagacggattgaatcaactttcgaatactttaatttggtcaagctcacacgaaactcatgtgagtgatcaaagtctgaaagccttctacctctgatttggatatgtacttaaaacactttctgagaaacaccctaacgcttggttggtggatttcacatcataacaattttagattttttattggtgatatgtggttttgtggttggagaagcagagttctaataactttatttcatcattattcacacaaaactgtattgtaccagtataggcattctcttcgataatacaaatatataatagtcaaaactttgctaattatcatatagtttcacatcatttgccaatacttttatacttattacgctaaatttttgtaggatttttttgattatttaggtttcatctcatttttaatatagaatttctaaaccgttttttttttcatttcatcatcttttctaaccattcatctttacagtctcttattatatatgattagtttatttcattcgtttgttcctttctatatattaaatttgtttcataataatcgaatttctatcaccatttgaagtctcaggttttgtgacatttggcattacgggaaaggtcccatgtatctcaaacttcttacacatgtggcaaaccatcagcctcgtgcacttgtaaaatcaaaaagttagtttatttacatggtttgtgtgatatttgagcttcctttcctacgtagttgtttgtgttaagttcaaaccaatggccatagctaacttggattcttcaaatatcttcttacttcatacatatgtctaacattgttgtttcatcttataagttattaactaaccacgtaaaaatgtttctacattgtattatagcaaaataaaccataacaatgtgaggatacaaattaaagttacaagttttcctcaaaattcaatcatacatcacactctcttacgtggttgcatacgtgtgtatatgcatttatgagagctaaagagtatccattcatttgcatatactttcatacgtttatatataactcttacttccttatgaaaccacaatatacactttctaatccaataattctctattgatacctttcaaatccccaa

SEQ ID NO: 8 - DNA, 1482 bp, Brassica napus
ataaccctctccatgttgataccaaatgtagtactgtggtgtaaatcctctgtttactaaatgcttccatacagtttcactacgtgcaaattttgaattcttgcatttccgacaggggcagaacatcttaccgctttcctgtgtgatcggtgtacagcccgcctggtgcatgaatgtctctagcccgctcagaaatgcgttcgtcaccctcccgtcggaatctttgtgcaaatacatccaaatccgtaactcgtaaatactaccgccaccggccatttttttcttagatttttttttgaaatctttttttttctgatttttttttcggatttttttttctcccgtttgtgtgttgtgaggaagagagttgtgggaaatgacatatatatagagaaattttcgagttgggtagttgaaatataacaacgattttataaggaatattttacatggattttacatggttttaacatattatttacaacgactttacgacgaaattaggtaagttaaagcacatttaatacacgttttcacctaaatataacggtaacatgcttcgttgtaatgtcgatgtaatgattacgacgtatttctcattccacgtacattcgtcgtaaacttacaaggagtttacgacgaaaccagttcgtcgtaaatttacatggcgtttacgacgaaagttagattcctcgtaatttcgttgtaaagcccatgtaaatttacgacgaaatattttcgtcgtaaatgttcgttgttatgggcacgttttcttgtagtgatttttccaactaaaaattattaaaaaaaagattaagaacctctaaatcagtatttgggataatgatgctcttattaggtatagttcttcgttggtagttgcattgtatcatcatttgattcaacgtggcatgcagttcattggctaattgttacacatctagcaaggctgctaattatacgtggtggtgagtacgtagttcaaggactaacctcgcagttgctcgtttttggtggatacttttgcataatcagttttgattgtgctatatctaaatctacgtgatgtaacgaccgatacaataaacaattctgattgaatatatatccatatgatagtatgacatgctgcgacgacaactatggtgttgccacgtaacgaccatctttattaaccatgacgtgccgcaaccacagaggacaacaacacatacagcatcgagaaaccgcatactaacactcgcaaagtgcaacacccaaaactcttaatttaattagcttaattctcagcaacaccacgcagctatacacgtatcttctatgctaacacgtgtcatgtcttgaaccgaccaagacacactacaaatgtatcgatggtttggagaagaccatacacacactttctttacacagcaacatacactttcagaaaaagctttatataattatcatttcatatttcacacctttgaagaaaagaaa

SEQ ID NO: 9 - DNA, 1564 bp, Brassica napus
actatagggcacgcgtggtcgacggcccgggctggttggtaaaaccacctcagcctccgattcagtttctggatccgaaaccgagaccgattccgaatccgataccaagaaggacaagtctccgccgccggcaaccaaatcgggaacgaagcggcccagcgaagggacttcgaaggaagcgaactcaaagcgagcgaagatcggcgaagactcgaagaagcctgctgcttttcagagactgtggaccgaggacgacgagatcgctgtcttacaaggcatgatcgatttcaagaaggatacggggaactctccttacgacgacaccaacgcttactacgattacatcaagaaatcaattagctttgaggttagcaagaaccagttcatggataagcttaggagtttgaagaagaagtatatgggcaaagagaagccttctttcaccaagcctcatgaccagaagtcttatagattgtgtaagtctatatggggacctgaggggatggttcttgagtccaacggcaagaagaccaagaaggttgtctctgtgaagcagcaagagctctcttttgcttcttctcccaatggtaaaacggttgatgatggtggtgacatgtatgattggtttgagaagtcgtttcttgtgcgagggattgcgggtttcggtgtcgatgagagttatgtgaaacagagatggaggttggtcccggtggagactaagaggaaagttgaagagaaggttaagatgttgcaggccaaggagattgagtttgtgttgcagaagactgagattttgcatgaagtcacctctttgattgctgaagcatctaagaacaagccattagaatgattgatttgatctacggtttcaatgccaattgtctatgcctctctattgttttttcaacttaggatttttactttttcttcttttatgatctacttactgtgtaatttttctggttcatttagatggctttctctcatatgaaactctcttatattggatttgttgctaatctataataacacaataaaaataagattactgtcaattgtcaagcctaaggcagttaggtgacatatgaactagatcctagctatggtcgaactgttgggtatttttatgaaatgtaggtaagtattggtttggttcgggtaatatgaacaaggttctagcaatttgtgtttaatcaaatgggtttgttctgaaaaattcaagtaaaaagatcaaaaatttggttaaagaagacgaacttacaaaaatcttgtgataacttagttaaatgataacaaatctgcaagtcaataaaaagttacgagtctttcttctgattcttaggtgagaatttaggaagaggaagaaggtgaacaagttagttgagtcacgtgcttaccttctaaaaagcctttttgatcaatggttgtaccaaatgagaagagagactataagcgttgcaacttttacttttacttttacttcctatataaaaagtctcaagacgttaaacctcataacaaacaaatataaatttctgtgcgttaagaatttataatatatcag

SEQ ID NO: 10 - DNA, 983 bp, Brassica napus
gttgatggaaatcgtatcgtcgtccaagtccctaccgctggctagaattgtatacgaacattatgtgtaatatagacttaagtatgtagacaatgtgtagctggttggtacgataataatgtagtttgcatcacctgcttaaatagtttgggtgtttgtttcactcagtattacttatgacttatgtctcagttatatctgtaaaccaagtaatttcggtgttcgttcaccaaatattacttacgtcccaaagttatatctataaagcaagtgatctgatatcctatgaaaatcaataatgtaatattcttctagtattaagtcttagggtgagcagagggttaattaaaacgctaaatcatcctcaagtcctgctgcgctcatcgagatgaaagagaacgaaaaaatattgtggaagatgattttagagaatatatttatatgaatgattgattaaaagaaatgttcaaaatgtataagcgtttcttctgagaaaatttacaaaattataaactttagccatgtacacagaatccattcagcattgggaatattctcttcacctaattctttttctttcaacattatctatgcttttgaaattttaacttgttgtcgatggatatgcttagagaaattttcaaaataaaattacttgtaagaacattaatcaagataagctagcatcagggaccggtgactgtccatccaatataatatatggttttaccagtactgatataatatatcgacaatttttaagaaaacggtataactttcaaagcttataggtttataaatgtcaaaggactgtatttcaaacccgttagtcaaggttttcgactttgaatacacaagcactcacctactacaccgacactgacattgacgtctctgattctataaattggactcagattcctaaagacgagcataacaaatacatcacagcactgcaaagataaaaaaaaaggggtagcaac

SEQ ID NO: 11 - DNA, 2243 bp, Brassica napus
ttgtactctcccttaatggagcttaaactcttctgaatttaatacaagttggtttgatcaaaaaaaaaaaaaaaagaaagacatgcagcgtacaatggtcagaataacaaagaaaattactacaatcgccagggaaaacgatttgacgtggtaagtattttcattggctagttctcacacgtataacaaggctaattatatgtggaggcgcatacattgatacatagtttaaactcttgttcgagaactcgttaggcgttcctgtttgggttggacttagagtataatgagaaaatcggaaattacgcggggattaattggggattagttttattgcatatataactatttaagggttatataggacatatgtgaaactaaactaacgataattcgtggtctagtggtatgcccacttcctaaagtccataggacacgggttacaacccgggctgaaccttttttcaactttttttttttaactaagtttaattaaagcaaaatgacaaaaatattcttttcttcgacctccacacctgcgattttacggccgctttttcactttttcttacgatttcttgatgtttttggtttataagttgatgtgaacatatattgaacagatcaatgatataatatgtgattatgggcttagaaacaatcgttttaaaaaaaatccgaagaactggccgaatcagtgcaaatcgccacggattgccgtcgcctcgcgcttttccggcgagttttcgaccgcgaacgcgttttcgaaaatgggtttaaactaaacatcgaagttgtctggatacttctatattttaaaagtatttcaaaaaaaaaaaacttctatattttaaatttctaagttttgatttcagctaactggtaaagaatattttaatgaacgatacttaggttcacctctgtggtgaatgattaaattcaccacacttttcataaccaatcaaagtgtcatgtaggagtagttaaaaaagacaataaaaataaaataaaaatagctgaaaaaaacaacaccgacattgattaacgtcgtttaaaaacctagactataaaaccataaatcctaaatctgaaacactaaaccataaatctcaatcctaaactctaaaccctaaatcttaaactttaaacccaaactctaaacaataaaccttaaatcctaaaatctaaaccctaaacccaaagctataaaccataaaccataaaatctaaaccctaaatcctaaatcctaaaccctaaactttttttttaaccctaaactcaaatcctcaactcaaatcctaaactctaaacccaaaacttcaaatcctaaacccaaacctctaaaccctaaacctatctgctagttaataagattaaggtttacggtttagaggtttgggtttatggtttatgatttagggtttgggtttaagatttaaagttttgggtttagggtttaggatttagagtttagattttaggatttagggcttattgtgtagagtttgggtttagggtttaagatttatggtttagattttaggattgggatttatggtttagtgttttggatttagggtttatggttttagagtttaggtttttagaatttagaattttgcggatcgcgctaattaatgtcggtgttgtttatttttttagctatttaattttttacttttattgtttttttaaactatttatatatgacattttggttggttatgaaaagtacggtgaatttaacattcaccgtagggtgaacctaagtattgttatattttaattgtataatgtccaagtgatgtttgattgtttgacaaggtcacggaaacatgcatgttacaaccgatacaatgatcaaaactgaacatagccatattgatattataacatgcagcaacacacttatggtgttgacacgtagcaagcatctttcagttaaccataacgtgtcacgacaacacaggataacacgtacaagatcgagaaaccgcatatactaaacactggcaaagtacaacacccatactcactaatttaattagcttttaatctccacacaccacgcagctatacacgtgtcttctatgccaacacgtgccttgttctcaaaccgaccaaggcacactataaatgtctcgatggtctggataacaatacacagtttatacacagcaacgaacatttacagaaaaagctttaactttcatttcctatttgcgcatttgaagaaagaaaa

SEQ ID NO: 12 - DNA, 1472 bp, Brassica napus
gtgaatcacaagcaaagagaaaccctaatacgaagaaaaaaacgattaatgataaagctggtctttgctctctcgctgccttcttcatttggaaggagggaagaaaatggcgtccgaagttctcttagaattacggaaagactcctccacctctcgatcaaccttaccattactgagaaaaagttaaaggagtaaaaccttaccattacctagggagactggtcctgaaacaaataaaacatatataaactgataaaacgtttgaatccaaccttataacaaaccaatctttacagttatagtgttatactatcgtctgttatacctctttgtcggggtccacaaaaatacactcaaaagtctgaaaagagaagttaaaggagtaaaaatagaagacataaaagaaaggaaacgaatgagccggaggaaccttaccgggtatttaacaacatggtggaaggagctaagtcttcttcaggaatcatatggaacacattccaatatcttcaaagaatctcactcatggattttatttatagcagtgagtttcaaattcccatctttccggaccgtttcacaaacatagcgaaagtattctacctgacgtaaaaaattgttttgaaatgatcaatcaaagtatcacatagaaaactatacaaataatatctatttttttacataatcattgttaagagtctattcatatttttttacttgtggacaaattcgtattattacttttcttgtttttgttttgttttgcactatcattttatttagatttcatatgtttaatttaattttctatttacaaaatattgacagctatttttatatttaattttggaaaattatcacagtagtgacaggtatactctactttttgtttgttttgttaactttctggataactataaattatttgcatattaaatttctagtcacaatagttatagatatattctactttttgtttgttttgttaactttctggataactacaaatcatttgcatatcaattttttagggaaattagtgctgtaaattttcaaaaaaaaatctatagataatctatgcattgatggtttattatgaacatatattttcattctttactagttaaatattatatgagattccttgcaaattttgatatttttcctattatgtagttttcttatatagaattgctttgaccaaaaaaaagaattgctaacatgtatcaaaatctggatgtatggatatacatatcttataataattaccaaagtaacatacctcacatagatacgaaaagcttacagtcaaaaattcaagatacactacctaaaaacgtcagccctcttacaaatattactccaacatccatctatataatagcttgactatcagaacctcaaaattaacttctcaaacatttctcagaaaaaaaactagaactaaggttgaagagtcgccaagcttacaaaac

SEQ ID NO: 13 - DNA, 1390 bp, Brassica napus
ccatggtggacctcaagcctggagtgaagcgcctggtgagctggaaggagatccgcgagcacgcgacgcccgcgaccgcgtggatcgtgattcaccacaaggtctacgacatctccaagtgggactcgcacccgggtggctccgtgatgctcacgcaggccggcgaggacgccacggacgccttcgcggtcttccacccgtcctcggcgctcaagctgctcgagcagttctacgtcggcgacgtggacgaaacctccaaggccgagatcgagggggagccggcgagcgacgaggagcgcgcgcgccgcgagcgcatcaacgagttcatcgcgtcctaccgccgtctgcgcgtcaaggtcaagggcatggggctctacgacgccagcgcgctctactacgcgtggaagctcgtgagcacgttcggcatcgcggtgctctcgatggcgatctgcttcttcttcaacagtttcgccatgtacatggtcgccggcgtgattatggggctcttctaccagcagtccggatggctggcgcacgacttcttgcacaaccaggtgtgcgagaaccgcacgctcggcaaccttatcggctgcctcgtgggcaacgcctggcagggcttcagcatgcagtggtggaagaacaagcacaacctgcaccacgcggtgccgaacctgcacagcgccaaggacgagggcttcatcggcgacccggacatcgacaccatgccgctgctggcgtggtctaaggagatggcgcgcaaggcgttcgagtcggcgcacggcccgttcttcatccgcaaccaggcgttcctatacttcccgctgctgctgctcgcgcgcctgagctggctcgcgcagtcgttcttctacgtgttcaccgagttctcgttcggcatcttcgacaaggtcgagttcgacggaccggagaaggcgggtctgatcgtgcactacatctggcagctcgcgatcccgtacttctgcaacatgagcctgtttgagggcgtggcatacttcctcatgggccaggcgtcctgcggcttgctcctggcgctggtgttcagtattggccacaacggcatgtcggtgtacgagcgcgaaaccaagccggacttctggcagctgcaggtgaccacgacgcgcaacatccgcgcgtcggtattcatggactggttcaccggtggcttgaactaccagatcgaccatcacctgttcccgctcgtgccgcgccacaacttgccaaaggtcaacgtgctcatcaagtcgctatgcaaggagttcgacatcccgttccacgagaccggcttctgggagggcatctacgaggtcgtggaccacctggcggacatcagcaaggaatttatcaccgagttcccagcgatgtaattaattaa